(19) United States
(12) Patent Application Publication
(10) Pub. No.: US 2016/0310417 A1
(43) Pub. Date: Oct. 27, 2016

(54) Formulations and Methods For Targeted Ocular Delivery of Therapeutic Agents

(71) Applicants: Emory University, Atlanta, GA (US); Georgia Tech Research Corporation, Atlanta, GA (US)

(72) Inventors: Mark R. PRAUSNITZ, Atlanta, GA (US); Yoo Chun KIM, Hendersonville, TN (US); Henry F. EDELHAUSER, Atlanta, GA (US)

(21) Appl. No.: 15/103,908

(22) PCT Filed: Dec. 19, 2014
(86) PCT No.: PCT/US2014/071623

§ 371 Date: Jun. 13, 2016

Related U.S. Application Data
(60) Provisional application No. 61/918,992, filed on Dec. 20, 2013.

(51) Int. Cl. A61K 9/00; A61K 31/5575; A61K 47/36; A61K 47/38; B23K 26/00; A61K 9/107; A61K 9/16; A61K 39/395; C07K 16/22; A61M 37/00; A61K 31/498; A61K 9/10

(52) U.S. Cl. CPC A61K 9/0051; A61K 31/498; A61K 31/5575; A61K 47/36; A61K 47/38; A61K 9/10; A61K 9/107; A61K 9/1611; A61K 9/1629; A61K 9/0021; A61K 39/3955; C07K 16/22; A61M 37/0015; B23K 26/0093; A61K 2039/505

(57) ABSTRACT

Formulations, systems, and methods of administration are provided for preferential targeted delivery of drug to ocular tissue. In embodiments, the formulation may include a non-Newtonian fluid that facilitates targeted localization or preferential spreading of the fluid formulation in the ocular tissue. The fluid formulation may be administered to an eye of a patient by inserting a microneedle into the eye at an insertion site, and infusing a volume of a fluid formulation through the microneedle into the suprachoroidal space of the eye at the insertion site over a first period. During the first period, the fluid formulation may be distributed over a first region which is less than about 10% of the suprachoroidal space, and during the second period subsequent to the first period the drug formulation may be distributed over a second region which is greater than about 20% of the suprachoroidal space.

### CROSS-REFERENCE TO RELATED APPLICATIONS

The present application claims benefit of U.S. Provisional Application No. 61/918,992, filed Dec. 20, 2013, the disclosure of which is incorporated herein by reference.

### STATEMENT REGARDING FEDERALLY SPONSORED RESEARCH OR DEVELOPMENT

This application was made with U.S. government support under contract nos. R24-EY017045 and R01-EY022097 from the National Eye Institute.

### BACKGROUND

Ocular diseases affect many people worldwide. It is estimated about 80 million people worldwide are visually impaired or disabled, and the number of patients increases approximately 7 million people per year. In United States alone, about 3.4 million people over the age of 40 are blind or visually impaired. Many ocular diseases can lead to blindness and are preventable if managed correctly.

Drug delivery into the eye poses significant challenges due to the complex anatomy and unique physiology of the eye. Most often, methods used to deliver drugs to both anterior and posterior of the eyes in clinic are topical, intravitreal, and periocular administrations. Topical delivery is the mainstay to deliver drugs to the anterior segment, but only acts transiently. Ocular barriers such as tear fluid, corneal epithelium, and conjunctiva only allow small amounts of applied drugs into the eye. Low penetration of the drug forces patients to follow stringent dosage regimens, which reduces patient compliance. Systemic (parenteral) administration could be used to target molecules to the other tissues to overcome the inefficiencies of the topical delivery; however, this non-targeted method requires a high dosage to deliver a therapeutically effective drug concentration, and both the blood-aqueous barrier and blood-retinal barrier express tight junctions that prevent the drugs from penetrating into the eye. Periocular administration delivers drugs on the outer surface of the eye for diffusion into the eye, offering minimal tissue damage but suffering from low targeting efficiency. Intravitreal injection, which involves administering the drug formulation directly into the center of the eye for it to diffuse outward towards the choroid and retina, is an invasive way to deliver drugs and often carries risk of ocular infections.

Microneedle-based ophthalmic drug delivery methods provide a promising tool for treatment of ocular diseases. Progress in this field, however, has been limited by the poorly targeted ability of suprachoroidal injection. Since the suprachoroidal space is right above the choroidal blood bed, drugs delivered to this region tend to be cleared rapidly from the suprachoroidal space. Injected polymeric particles tend to cover only a portion of the suprachoroidial space, but are not well targeted either anteriorly to the ciliary body or posteriorly to the whole layer of the choroid. For example, a high pressure point at the back of the eye makes it hard for injected particles to penetrate towards the back of human eyes. Meanwhile, an anteriorly injected formulation quickly spreads away from the injection site when the ciliary body is targeted. Thus, existing methods may have only limited success preferentially administering a drug to a target tissue within the eye.

Hence, there is great need for improved formulations and methods for administering drug to the eye. The effective drug delivery system should be (i) minimally invasive, (ii) safe, and (iii) selectively targeted. Minimal invasiveness reduces any damage to the ocular tissue, possible infections and pain associated with delivery, which increases patient compliance. Highly targeted drug delivery methods also may allow for administration of significantly reduced amounts of drug by efficiently delivering a high amount of the drug at the targeted site, thereby reducing possible deleterious side effects. Highly targeted delivery also may allow for development of controlled release formulations that would not otherwise be effective due to the low penetration of many ophthalmic drugs.

### SUMMARY

In one aspect, a fluid formulation is provided for administration to a suprachoroidal space of an eye of a patient. The formulation may include particles comprising a therapeutic agent and a non-Newtonian fluid in which the particles are dispersed, providing a formulation with a low shear rate viscosity from about 50 to about 275,000 cP. The formulation is effective to permit migration of the particles from an insertion site in the suprachoroidal space to a treatment site, which is distal to the insertion site, in the suprachoroidal space, and facilitates localization of the particles at the treatment site in the suprachoroidal space.

In another aspect, a method is provided for administering a therapeutic agent to an eye of a patient. The method may include inserting a microneedle into the eye at an insertion site and infusing a volume of a fluid formulation through the microneedle into the suprachoroidal space of the eye at the insertion site over a first period. The fluid formulation may include particles, a polymeric continuous phase in which the particles are dispersed, and a therapeutic agent which is in the particles and/or in the continuous phase, and may have a low shear rate viscosity from about 50 cP to about 275,000 cP. During the first period, the fluid formulation may be distributed over a first region which is less than about 10% of the suprachoroidal space, whereas during a second period subsequent to the first period, the fluid formulation may be distributed over a second region which is greater than about 20% of the suprachoroidal space.

In another embodiment of preferentially administering a therapeutic agent to an eye of a patient, the method may include inserting a microneedle into the eye at an insertion site and infusing a volume of a fluid formulation through the microneedle into the suprachoroidal space of the eye at the insertion site over a first period. The fluid formulation may include microparticles having a specific gravity greater than or less than 1, and a continuous phase in which the microparticles are dispersed, the therapeutic agent being in the microparticles and/or in the continuous phase. The method further includes preferentially targeting a tissue by positioning the patient in the gravitational field so that the microparticles move either upward or downward in the gravitational field depending on the specific gravity of the microparticles.

In another embodiment, a method is provided for treating glaucoma by administering a drug formulation to an eye of a patient, wherein the method includes inserting a microneedle into the eye at an anterior portion of the eye and then infusing a volume of a drug formulation through the microneedle into the suprachoroidal space of the eye at the insertion site. The fluid formulation includes particles, a polymeric continuous phase in which the particles are dispersed, and a therapeutic agent which is in the particles and/or in the continuous phase. The drug formulation has a low shear rate viscosity of greater than about 10,000 cP such that the drug formulation is substantially localized at the insertion site after being infused into the suprachoroidal space.

### BRIEF DESCRIPTION OF THE DRAWINGS

FIG. 1A shows a high magnification of one example of a hollow microneedle. FIG. 1B shows a hollow microneedle mounted on a luer adapter attached to a syringe. FIG. 1C provides a comparison of the relative size of a microneedle and a liquid drop from a conventional eye dropper.

FIG. 2A is a schematic diagram showing a particle stabilized emulsion droplet (PED) with a perfluorodecaline liquid core and a surface coated with polymeric nanoparticles, which stabilize the interface and serve as model particles to encapsulate drug for controlled release delivery. FIGS. 2B-2E are a schematic illustration of administration of PEDs to an eye of a patient by injection into the suprachoroidal space of the eye (2B), resulting in initial distribution over a large area of the space (2C), falling to the back of the eye due to gravity (2D), and remaining substantially localized at the back of the eye after the aqueous carrier fluid is cleared (2E).

FIG. 3 is a graph quantifying the amount of bevacizumab coated onto microneedles and delivered into the cornea, comparing the measured coating amount (μg), calculated amount delivered (μg), measured amount left on the needle (μg), and measured amount in tear fluid after the injection (μg). Data show average±SEM (n=4 replicates).

FIGS. 4A and 4B are graphs quantifying corneal neovascularization after suture-induced injury and treatment with bevacizumab by topical and intrastromal routes over time (4A) and compared between neovascularization area at days 10 and 18 (4B) for four treatment groups: untreated (UT), microneedle placebo (MN-placebo), topical delivery of bevacizumab (TOP) and bevacizumab bolus given by four microneedles (MN-4bolus). The * symbol indicates a significant difference compared to the untreated group (p<0.05); The [symbol] symbol indicates a significant difference compared to the topical delivery (TOP) group (p<0.05). Data show average±SEM (n=5-6).

FIGS. 5A and 5B are graphs quantifying corneal neovascularization after suture-induced injury and treatment with bevacizumab by subconjunctival and intrastromal routes over time (5A) and compared between neovascularization area at days 10 and 18 (5B) for four treatment groups: untreated (UT), bevacizumab administered as a bolus on day 4 by low-dose subconjunctival injection (SC-low), high-dose subconjunctival injection (SC-high) and intrastromal delivery using four microneedles (MN-4bolus). The * symbol indicates a significant difference compared to the untreated group (p<0.05); Data show average±SEM (n=5-6).

FIGS. 6A and 6B are graphs quantifying corneal neovascularization after suture-induced injury and treatment with bevacizumab as a function of dose by intrastromal routes over time (6A) and compared between neovascularization area at days 10 and 18 (6B) for five treatment groups: untreated (UT) and intrastromal delivery of 1.1 μg on day 4 (MN-1bolus), 1.1 μg on days 4, 6 and 8 (MN-1bolus×3), 4.4 μg on day 4 (MN-4bolus) and 50 μg on day 4 (MN-hollow). The * symbol indicates a significant difference compared to the untreated group (p<0.05); Data show average±SEM (n=4-6).

FIGS. 7A and 7B are graphs showing the effect of topical sulprostone (7A) or topical brimonidine (7B) administration on IOP in the rabbit eye. A single drop containing 2.5 μg sulprostone (7A) or 75 μg brimonidine (7B) was administered to one eye. IOP was then followed for 9 hours in both the treated eye and the untreated/contralateral eye. Data points represent the average±SEM (n=4-5).

FIG. 8 is a graph showing the effect of supraciliary injection on IOP in the rabbit eye with a single injection of 10 μl of a 2% w/v solution of CMC administered to one eye. IOP was then followed for 9 hours in both the treated eye and the untreated/contralateral eye. Data points represent the average±SEM (n=3).

FIGS. 9A and 9B are graphs showing the effect of supraciliary injection of sulprostone on IOP in the rabbit eye for a single injection of 0.025 μg (9A) or 0.005 μg (9B) sulprostone in 10 μL administered to one eye. IOP was then followed for 9 hours in both the treated eye and the untreated/contralateral eye. Data points represent the average±SEM (n=4-6).

FIG. 10A is a graph comparing the IOP drop caused by supraciliary delivery versus topical delivery of sulprostone, including data from FIGS. 7A and 9 graphed together to show the dose-response relationship after supraciliary delivery and to facilitate comparison with topical delivery in the treated eyes. FIG. 10B is a graph comparing the pharmacodynamic area under the curve (AUC_{PD}) after supraciliary delivery in treated and contralateral eyes, and in comparison with topical delivery, including data from FIGS. 7A and 9 and calculated using Equation (1).

FIGS. 11A-11C are graphs showing the effect of supraciliary injection of brimonidine on IOP in the rabbit eye for a single injection of 1.5 μg (11A), 0.75 μg (11B), and 0.015 μg (11C) brimonidine in 10 μL administered to one eye. IOP was then followed for 9 hours in both the treated eye and the untreated/contralateral eye. Data points represent the average±SEM (n=3-5).

FIG. 12A is a graph comparing IOP drop caused by supraciliary delivery versus topical delivery of brimonidine including data from FIGS. 7B and 11 graphed together to show the dose-response relationship after supraciliary delivery and to facilitate comparison with topical delivery in the treated eyes. FIG. 12B is a graph comparing the pharmacodynamic area under the curve (AUC_{PD}) after supraciliary delivery in treated and contralateral eyes, and in comparison with topical delivery, including data from FIGS. 7B and 11 and calculated using Equation (1).

FIG. 13 is a graph comparing the IOP increase due to injection of 50 μl of Hank's Balanced Salt Solution (BSS) into the intravitreal space (IVT) and 10 μL and 50 μL of 2% carboxymethylcellulose placebo formulation (CMC) into the supraciliary space (SCS).

FIGS. 14A-14C are representative confocal microscope images of 14 μm (14A), 25 μm (14B), and 35 μm (14C) diameter PEDs. The scale bar indicates 40 μm. FIG. 14D is a Brightfield image of 35 μm PEDs immediately after vigorously shaking the vial (left) and 30 seconds later (right).

FIGS. 15A and 15B are graphs showing gravity-mediated delivery of PEDs in the rabbit eye ex vivo by distribution of particles away from the ciliary body for two different orientations (cornea down and up) (15A) and radial distribution of particles away from the injection site (at superior “12-o'clock” position) (15B). Asterisk (*) indicates statistical significance between two different orientations. Data shown as average±standard deviation (n=3-5 replicates).

FIGS. 16A and 16B are graphs showing lack of gravitational effect on delivery of polystyrene microparticles in the rabbit eye in vivo (cornea facing up) by distribution of particles away from ciliary body (16A) and radial distribution of particles away from the injection site (at superior “12-o'clock” position) (16B) for polystyrene microparticles and PEDs. Asterisk (*) indicates statistical significance between polystyrene microparticles and PEDs. Data shown as average±standard deviation (n=3).

FIGS. 17A and 17B are graphs showing the retention of PEDs at the site of targeted delivery by distribution of particles away from the ciliary body (17A) and radial distribution of particles away from the injection site (at superior “12-o'clock” position) (17B). Asterisk (*) indicates statistical significance between day 0 and day 5. Data shown as average±standard deviation (n=3).

FIG. 18 is a graph comparing the effect of PED size on gravity-mediated targeting of 14 μm, 25 μm, and 35 μm diameter particles after injection in the rabbit eye in vivo by radial distribution of particles away from the injection site (at superior “12-o'clock” position). Data shown as average±standard deviation (n=3).

FIG. 19 is a graph showing the kinetics of suprachoroidal space collapse by the intraocular pressure change after injecting 200 μL of BSS into the suprachoroidal space of the rabbit eye in vivo. Data shown as average±standard deviation (n=2).

FIGS. 20A-20C are a brightfield image of flat mounted eye (20A), a florescent image of the red fluorescent particles in the eye (20B), and a fluorescent image of near-infrared particles in the eye (20C).

FIG. 21A is a graph showing the suprachoroidal surface coverage area as function of time and particle size. FIG. 21B is a graph showing the mass of fluorescent particles in the suprachoroidal space as a function of time and particle size. Asterisk (*) indicates statistical difference between days 14 and 112.

### DETAILED DESCRIPTION

Novel formulations, systems, and methods are provided for addressing the needs described above and providing preferential administration of materials to specific locations within the eye. Although most of the disclosure makes reference to delivery of materials, methods for removal of tissue or fluid also are envisaged.

In certain embodiments, the delivery methods and drug formulations take advantage of the temporary expansion of the suprachoroidal space (SCS) following fluid infusion into the space. That is, the drug formulations beneficially are designed to control migration of the drug, particles, and other materials within the SCS in the limited period while the space is expanded following fluid infusion. In some cases, this means that the mobility of the infused formulation (or part thereof) within the space is facilitated, and in other cases, it is retarded, for example by controlling rheological characteristics of the formulation as detailed herein.

Unless otherwise defined herein, all technical and scientific terms used herein have meanings commonly understood by those of ordinary skill in the art to which the present invention belongs. It is also to be understood that the terminology used herein is for the purpose of describing particular embodiments only, and is not intended to be limiting. In describing and claiming the present invention, the following terminology will be used in accordance with the definitions set out below.

As used in this specification and the appended claims, the singular forms “a,” “an,” and “the” include plural referents unless the content clearly dictates otherwise. Thus, for example, reference to “a component” can include a combination of two or more components; reference to “a buffer” can include mixtures of buffers, and the like.

As used herein, the terms “comprise,” “comprising,” “include,” and “including” are intended to be open, non-limiting terms, unless the contrary is expressly indicated.

The term “about,” as used herein, indicates the value of a given quantity can include quantities ranging within 10% of the stated value, or optionally within 5% of the value, or in some embodiments within 1% of the value, or in some embodiments within 0.1% of the value. For example, about 0.5 may include about 0.45 and 0.55, about 10 may include 9 and 11, about 1000 may include 900 to 1100.

As used herein, the terms “proximal” and “distal” refer to a position that is closer to and away from, respectively, a relative position. For example, an operator (e.g., surgeon, physician, nurse, technician, etc.) inserting the microneedle device into the patient would insert the tip-end portion of the microneedle device into the ocular tissue first. Thus, the tip-end portion of the microneedle would be referred to as the distal end, while the opposite end of the microneedle (e.g., the base or end of the microneedle device being manipulated by the operator) would be the proximal end.

In exemplary embodiments, targeted delivery of a material is achieved by administration of a fluid formulation that is formulated to (i) minimize the spread of the fluid formulation from the insertion site, (ii) maximize and/or control the spread of the fluid formulation from the insertion site, (iii) preferentially spread upon application of one or more external forces, and/or (iv) maximize the delivery efficiency of the material to the target tissue. The material may be released into the ocular tissues from the fluid formulation over a specified period (e.g., either during insertion of the microneedle or over an extended period after the microneedle has been inserted and withdrawn). This beneficially can provide increased bioavailability of the material relative, for example, to delivery by topical or systemic application and without the deleterious effects of more invasive intravitreal injections.

The material to be delivered generally is referred to herein as a “drug,” “medicament,” or “therapeutic agent.” These terms are being used for convenience and as exemplary materials in the fluid formulation for delivery via the microneedle device. Thus, reference to exemplary materials is not intended to limit the material in the fluid formulations to drugs, for example, but rather is representative of any material that may be delivered to an ocular tissue using a microneedle device. Similarly, when the material to be delivered includes microparticles or nanoparticles, the term “particles” is used for convenience to refer to microparticles, nanoparticles, or combinations thereof.

Generally described, the fluid formulations provided herein may be administered by injecting (inserting) a microneedle into an insertion site in the ocular tissue. The microneedle allows for precise control of the depth and site of insertion into the ocular tissue, enabling the administration of the fluid formulation in a minimally invasive manner that is superior to conventional needle approaches. For instance, the microneedle may be inserted into the anterior segment of the eye (i.e., the portion of the eye that is more readily accessible) for preferential and targeted delivery of the fluid formulation to one or more locations within one or both of the anterior segment and the posterior segment. In certain embodiments, the microneedle is inserted into the ocular tissue at a site suitable for administration of the fluid formulation via the SCS for targeted delivery to one or more target tissues.

As used herein, the term “suprachoroidal space,” or SCS, which is synonymous with suprachoroid or suprachoroidia, describes the potential space in the region of the eye disposed between the sclera and choroid. This region primarily is composed of packed layers of long pigmented processes derived from each of two adjacent tissues; however, a space can develop in this region as a result of fluid or other material buildup in the suprachoroidal space and the adjacent tissues. The “supraciliary space,” as used herein, refers to the most anterior portion of the suprachoroidal space adjacent to the ciliary body, trabecular meshwork and limbus.

Formulation

The formulation generally may be a fluid formulation in the form of a liquid drug, a liquid solution that includes a drug in a suitable solvent, liquid suspension, or liquid emulsion. The liquid suspension may include particles dispersed in a suitable liquid vehicle for infusion. In various embodiments, the drug is included in the liquid vehicle, in the particles, or in both the vehicle and particles. In some embodiments, the formulation is associated with the microneedles as either a coating on solid microneedles or encapsulated in solid microneedles. Advantageously, the formulation is specially formulated to control the spread of the formulation during and/or after injection of the formulation into the ocular tissue.

For example, in embodiments, the spread of the formulation is controlled by modifying the volume of the formulation such that the spread of the formulation during and/or after injection of the formulation into the ocular tissue is either minimized or maximized, depending on whether the target tissue(s) is/are at or near the site of insertion (i.e., proximal to the site of insertion) or away from the site of insertion (i.e., distal to the site of insertion). In embodiments, the volume of formulation for administration can be reduced to less than 50 μL, 20 μL, 10 μL, 5 μL, or 1 μL, in order to localize a majority of the drug at the treatment site (i.e., reducing the spread of the formulation). Conversely, in embodiments, the volume of formulation for administration can be increased to greater than about 100 μL, 150 μL, 200 μL, 300 μL, 400 μL, or 500 μL, in order to maximize spreading of the formulation.

In embodiments, the viscosity of the formulation when in its fluid form is used to control the spread of the formulation during and/or after injection of the formulation into ocular tissue. For example, the formulation may be configured to substantially evenly distribute the drug throughout a majority of the SCS, to localize a majority of the drug at the treatment site, to substantially localize a majority of the drug at the injection site, or to control the spreading of the formulation as a function of time. In an exemplary embodiment, the formulation is configured to reduce spreading of the formulation at the insertion site during an initial time period while increasing spreading of the formulation during a subsequent, later time period.

Generally, the viscosity of the formulation when in its fluid form may be increased to minimize spread of the formulation during injection. Although increasing the viscosity may limit spread after injection, it also will make it more difficult to inject the formulation through the microneedle. For this reason, it may be advantageous to use a fluid formulation that is a non-Newtonian fluid (i.e., that is thixotropic or shear-thinning). Non-Newtonian fluids generally are characterized by a viscosity dependence on shear force, such that application of a high shear rate reduces the apparent viscosity and application of a low shear rate increases the viscosity. As used herein, a “high shear rate” or “high shear rate viscosity” refers to a viscosity measured at 10 s⁻¹, 100 s⁻¹, or 1000 s⁻¹, and a “low shear rate” or “low shear rate viscosity” refers to a viscosity measured at 0.1 s⁻¹, 0.01 s⁻¹, or 0.001 s⁻¹. In that way, the viscosity can be higher after injection into the tissue (e.g., because the shear force in the suprachoroidal space is lower) and lower during injection through the microneedle (e.g., because the shear force is higher due to the small channel size in the microneedle).

In embodiments, the non-Newtonian fluid of the formulation has an apparent viscosity during injection through the microneedle (i.e., a high shear rate viscosity) from about 2 cP to about 1000 cP (centiPoise), about 5 cP to about 500 cP, about 10 cP to about 100 cP, or about 20 cP to about 50 cP. The non-Newtonian fluid of the formulation may have a low shear rate viscosity of at least 1000 cP, 2000 cP, 5000 cP, 10,000 cP, 20,000 cP, 50,000 cP, 100,000 cP, 200,000 cP, 500,000 cP, or 1,000,000 cP. Thus, the non-Newtonian fluid of the formulation may be characterized by a ratio of a low shear rate viscosity to a high shear rate viscosity of at least 5, 10, 20, 50, 100, 200, 500, or 1000.

The preferential delivery of the formulation to the ocular tissue depends at least in part on the viscosity of the non-Newtonian fluid of the formulation. Generally, localization of the formulation may be attained using a non-Newtonian fluid with a low shear rate viscosity of at least 10,000 cP, at least 100,000 cP, at least 300,000 cP, at least 500,000 cP, or at least 1,000,000 cP. In embodiments in which substantial localization of the formulation is desired, a more strongly non-Newtonian fluid may be preferred.

In many cases, the higher the low shear rate viscosity, the more localized the formulation upon injection, and the longer the formulation remains localized over time. Thus, in some cases, localization of the formulation for a period of time on the order of hours or days (e.g., for at least one hour, two hours, six hours, 12 hours, 24 hours, 48 hours) is the objective or is sufficient. In other cases, localization of the formulation for a longer period of time (e.g., for at least three days, five days, seven days, 10 days, 14 days, three weeks, four weeks, one month, six weeks, two months, three months, four months, six months) is the objective or is sufficient.

For more weakly or moderate non-Newtonian fluids, however, an increased viscosity at low shear rate may only limit spreading of the formulation for a limited period while promoting spreading of the formulation over a subsequent period. Thus, in one embodiment, a formulation is desired that decreases spreading of the fluid formulation over an initial period and increases spreading of the formulation over a subsequent period. Non-limiting examples of such formulations may include a non-Newtonian fluid having a viscosity at low shear rates of less than about 500,000 cP. For example, the viscosity at low shear rate may be from about 2 cP to about 500,000 cP, from about 50 cP to about 300,000 cP, from about 100 cP to about 275,000 cP, from about 500 cP to about 250,000 cP, from about 1,000 cP to about 200,000 cP, or from about 5,000 to about 100,000 cP.

The viscosity of these formulations also may be characterized by the slope on a viscosity versus shear rate graph of greater than (i.e., less steep than) −10,000 cP/s⁻¹, −5,000 cP/s⁻¹, −2,000 cP/s⁻¹, −1,000 cP/s⁻¹, −500 cP/s⁻¹, −200 cP/s⁻¹, −100 cP/s⁻¹, −50 cP/s⁻¹, −20 cP/s⁻¹, −10 cP/s⁻¹ between a shear rate of about 0.1 s⁻¹ and about 0.01 s⁻¹ or about 0.01 s⁻¹ and about 0.001 s⁻¹. For avoidance of doubt, because the slope has a negative value, a slope greater than one of the values indicated would be a less negative number or, stated another way, would be a smaller number on an absolute value basis (e.g., a slope of −100 cP/s⁻¹ would be greater than a slope of −1,000 cP/s⁻¹).

The viscosity of these formulations may be dependent at least in part on the presence of one or more pharmaceutically acceptable excipient materials in the formulation. As used herein, the term “excipient” refers to any non-active ingredient of the formulation intended to facilitate handling, stability, dispersibility, wettability, release kinetics, and/or injection of the drug. For example, the formulation may comprise drug-containing particles suspended in an aqueous or non-aqueous liquid vehicle (excipient), the liquid vehicle being a pharmaceutically acceptable aqueous solution that optionally further includes a surfactant. In some embodiments, particles of drug themselves may include an excipient material, such as a polymer, a polysaccharide, a surfactant, etc., which are known in the art to control the kinetics of drug release from particles and which may be used to modulate the viscosity of the formulation.

In exemplary embodiments, the formulation includes a polymer excipient capable of imparting the rheological properties to the formulation needed for preferential administration of the formulation to the ocular tissue. For example, polymer excipients such as methyl cellulose, carboxymethyl cellulose, and hyaluronic acid may be particularly suitable at imparting the desired rheological properties to the formulation, depending on both the concentration and the molecular weight of the polymer excipient.

In an exemplary embodiment of the formulation which decreases spreading of the formulation over an initial period and increases spreading of the formulation over a subsequent period, the formulation includes a weakly non-Newtonian fluid, particularly those weakly non-Newtonian fluids with a high molecular weight polymer excipient. For example, in embodiments the weakly non-Newtonian fluid includes a carboxymethyl cellulose having a molecular weight from about 90 kDa to about 700 kDa, a methylcellulose having a molecular weight from about 50 kDa to about 100 kDa, a hyaluronic acid having a molecular weight from about 100 kDa to about 1000 kDa, or a combination thereof. In one embodiment, the weakly non-Newtonian fluid includes a hyaluronic acid with a molecular weight from about 250 kDa to about 950 kDa, from about 250 kDa to about 750 kDa, or from about 500 kDa to about 750 kDa at a concentration from about 0.001% to about 5% weight/volume. For example, a commercially available product including both sodium hyaluronate and chondroitin sulfate, such as DisCoVisc® (Alcon Laboratories, Inc., Fort Worth, Tex., USA), may be used at one to four times the clinical concentration. In another embodiment, the weakly non-Newtonian fluid comprises a carboxy methylcellulose having a molecular weight of about 90 kDa to about 500 kDa at a concentration from about 0.5% to about 3% weight/volume. In another embodiment, the weakly non-Newtonian fluid comprises a methylcellulose having a molecular weight of about 90 kDa at a concentration from about 1% to about 3.5% weight/volume.

The above-described formulations may include a wide range of drugs for delivery to ocular tissues. As used herein, the term “drug” refers to a suitable prophylactic, therapeutic, or diagnostic agent, i.e., an ingredient useful for medical applications. The drug may be an active pharmaceutical ingredient. For example, the drug may be selected from small molecules or suitable proteins, peptides and fragments thereof, which can be naturally occurring, synthesized or recombinantly produced, including antibodies and antibody fragments (e.g., a Fab, Fv or Fc fragment). For example, the drug may be a small molecule drug, an endogenous protein or fragment thereof, or an endogenous peptide or fragment thereof. The drug may be selected from suitable oligonucleotides (e.g., antisense oligonucleotide agents), polynucleotides (e.g., therapeutic DNA), ribozymes, dsRNAs, siRNA, RNAi, gene therapy vectors, and/or vaccines for therapeutic use. The drug may be an aptamer (e.g., an oligonucleotide or peptide molecule that binds to a specific target molecule).

Representative examples of types of drugs for delivery to ocular tissues include antibiotics, antiviral agents, analgesics, anesthetics, antihistamines, anti-inflammatory agents, immunosuppressives, T-cell inhibitors, alkylating agents, biologics, and antineoplastic agents. Non-limiting examples of specific drugs and classes of drugs include β-adrenoceptor antagonists (e.g., carteolol, cetamolol, betaxolol, levobunolol, metipranolol, timolol), miotics (e.g., pilocarpine, carbachol, physostigmine), sympathomimetics (e.g., adrenaline, dipivefrine), calcium channel blockers, antimetabolites (e.g., carboplatin, episodium, vinblastine), carbonic anhydrase inhibitors (e.g., acetazolamide, dorzolamide), prostaglandins, anti-microbial compounds, including anti-bacterials and anti-fungals (e.g., chloramphenicol, chlortetracycline, ciprofloxacin, framycetin, fusidic acid, gentamicin, neomycin, norfloxacin, ofloxacin, polymyxin, propamidine, tetracycline, tobramycin, quinolines), anti-viral compounds (e.g., acyclovir, cidofovir, idoxuridine, interferons), aldose reductase inhibitors, anti-inflammatory and/or anti-allergy compounds (e.g., steroidal compounds such as triamcinolone, betamethasone, clobetasone, dexamethasone, fluorometholone, hydrocortisone, prednisolone and non-steroidal compounds such as antazoline, bromfenac, diclofenac, indomethacin, lodoxamide, saprofen, sodium cromoglycate), artificial tear/dry eye therapies, local anesthetics (e.g., amethocaine, lignocaine, oxbuprocaine, proxymetacaine), cyclosporine, diclofenac, urogastrone and growth factors such as epidermal growth factor, mydriatics and cycloplegics, mitomycin C, and collagenase inhibitors and treatments of age-related macular degeneration such as pegagtanib sodium, ranibizumab, bevacizumab, and afilbercept.

In certain embodiments, the drug is an anti-glaucoma agent, such as prostaglandins including the active ingredients in Xalatan (Pfizer), Lumigan (Allergan), Travatan Z (Alcon) and Rescula (Novartis); beta-blockers, including the active ingredients in Timoptic XE (Merck), Istalol (ISTA) and Betoptic S (Alcon); alpha-adrenergic agonists, including the active ingredients in Iopidine (Alcon), Alphagan (Allergan), and Alphagan-P (Allergan); carbonic anhydrase inhibitors, including the active ingredients in Trusopt (Merck), Azopt (Alcon), Diamox (Sigma), Neptazane (Wyeth-Ayerst) and Daranide (Merck, Sharp, & Dohme), parasympathomimetics, including pilocarpine, carbachol, echothiophate and demecarium; epinephrine, including epinephrine and dipivalyl epinephrine; and the active ingredients in marijuana.

In certain embodiments, the drug is an integrin antagonist, a selectin antagonist, an adhesion molecule antagonist (e.g., Intercellular Adhesion Molecule (ICAM)-1, ICAM-2, ICAM-3, Platelet Endothelial Adhesion Molecule (PCAM), Vascular Cell Adhesion Molecule (VCAM), or lymphocyte function-associated antigen 1 (LFA-1)), a basic fibroblast growth factor antagonist, or a leukocyte adhesion-inducing cytokine or growth factor antagonist (e.g., Tumor Neucrosis Factor-α (TNF-α), Interleukin-1β (IL-1β), Monocyte Chemotatic Protein-1 (MCP-1), Platelet-Derived Growth Factor (PDGF), and a Vascular Endothelial Growth Factor (VEGF)). For example, in embodiments the drug is an integrin antagonist that is a small molecule integrain antagonist, such as that described by Paolillo et al. (Mini Rev Med Chem, 2009, vol. 12, pp. 1439-46) or a vascular endothelial growth factor, as described in U.S. Pat. No. 6,524,581. In certain other embodiments, the drug is sub-immunoglobulin antigen-binding molecules, such as Fv immunoglobulin fragments, minibodies, and the like, as described in U.S. Pat. No. 6,773,916 to Thiel, et al. In one embodiment, the drug is a humanized antibody or a fragment thereof. In another embodiment, the drug is a diagnostic agent, such as a contrast agent.

In one embodiment, the drug is incorporated within particles that contain the drug and may control its release. Advantageously, the non-Newtonian fluid formulations provided herein can be especially useful to facilitate preferential delivery of the particles to the ocular tissue. The particles may be microparticles, nanoparticles, or combinations thereof. As used herein, the term “microparticle” encompasses microspheres, microcapsules, microparticles, and beads, having a number average diameter of about 1 μm to about 100 μm, about 5 μm to 50 μm, about 10 μm to about 40 μm, about 20 μm to about 35 μm, or about 30 μm to about 35 μm. The term “nanoparticles” refers to particles having a number average diameter of 1 nm to 1000 nm. The particles may or may not be spherical in shape. In some embodiments, the particles may be “capsules,” which are particles having an outer shell surrounding a core of another material. The core can be liquid, gel, solid, gas, or a combination thereof. In one case, the capsule may be a liposome. In another case, the capsule may be a “bubble” having an outer shell surrounding a core of gas, wherein the drug is disposed on the surface of the outer shell, in the outer shell itself, or in the core. In some embodiments, the particles may be “spheres,” which include solid spheres that optionally may be porous and include a sponge-like or honeycomb structure formed by pores or voids in a matrix material or shell, or can include multiple discrete voids in a matrix material or shell. The particles may further include a matrix material, which may provide for controlled, extended, or sustained release of the drug. The shell or matrix material may be a polymer, amino acid, saccharide, or other material known in the art of microencapsulation.

In particular embodiments, the particles are formulated to have one or more characteristics that facilitate preferentially directing migration of the particles by application of one or more external forces. For example, particles with a density that is different from that of water (e.g., a specific gravity of greater than or less than 1.0) may be preferentially directed using gravity. In one embodiment, the particles have a specific gravity greater than 1.0, 1.2, 1.5, 1.7, 2.0, 2.5, or 3.0, where the goal is to preferentially direct the particles in the direction of the gravitational field. In another embodiment, the particles have a specific gravity of less than 1.0, 0.9, 0.8, 0.7, 0.5, 0.3, 0.2, or 0.1, where the goal is to preferentially direct the particles in the direction opposite the gravitation field. The specific gravity of the particles may be controlled by forming the particles using a high- or low-density material in the core. Non-limiting examples of suitable high-density materials include liquids and solids, fluorocarbons, such as perflurodecalin, salts, such as calcium phosphates, polymers, such as crospovidone, metals such as ferric oxides, and glycerols. Non-limiting examples of suitable low-density materials include liquids and gases, such as air, nitrogen and argon, fluorocarbons, alcohols, such as ethanol and cetyl alcohol, and oils.

In embodiments, the particles include other features that facilitate preferentially directing migration of the particles by application of other types of external forces. For example, in embodiments the particles may include an electrical charge that may be moved within an electric field, or may be stably or inducibly magnetic to be moved in a magnetic field. In such embodiments, it is desirable that the particles be large enough to promote movement of the particles upon application of the external force, but small enough to be injected into the ocular tissue and migrate through the ocular tissue without significant hindrance. For example, when injecting particles via a microneedle, it may be desirable to use particles having a diameter greater than about 1 μm, about 5 μm, about 10 μm, about 15 μm, about 20 μm, about 25 μm, about 30 μm, about 35 μm, about 40 μm, or about 50 μm.

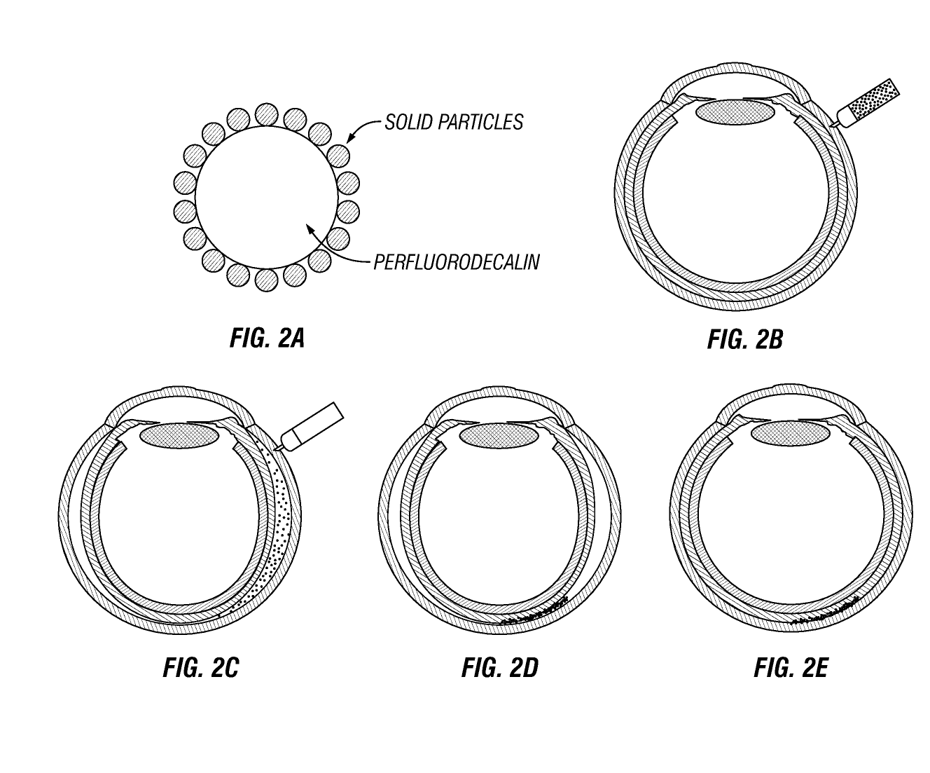

In an exemplary embodiment, the particles include particle-stabilized emulsion droplets. As used herein, “particle-stabilized emulsion droplets” or “PEDs” refers to a high-density liquid core surrounded about its edges by nanoparticles, illustrated in FIG. 2A. The nanoparticles function to both carry encapsulated drugs and to stabilize the emulsion interface to prevent coalescence into larger droplets (i.e., by forming a Pickering emulsion). Stabilization of the emulsion droplets may be achieved at least in part by controlling both the hydrophilicity of the nanoparticles (e.g., such that the nanoparticles prefer to be at the emulsion droplet interface and not in either the fluid formulation or liquid core). In addition, it may be desirable to use larger nanoparticles in PEDs, as the larger nanoparticles may provide longer controlled release. Thus, in embodiments the nanoparticles may be from about 10 nm to about 200 nm.

In one embodiment, the formulation further includes an agent effective to degrade collagen or glycosaminoglycan (i.e., GAG) fibers in the sclera, which may enhance penetration/release of the drug into the ocular tissues. This agent may be, for example, an enzyme, such a hyaluronidase, a collagenase, or a combination thereof. In a variation of this method, the enzyme is administered to the ocular tissue in a separate step from—preceding or following—infusion of the drug. The enzyme and drug are administered at the same site.

In some embodiments, the formulation changes properties upon delivery to the ocular tissue. For example, a formulation in the form of a liquid may gel or solidify within the ocular tissue. The gelation or solidifying of such a formulation upon delivery into the ocular tissue may be mediated, for example, by the presence of water, removal of solvent, change of temperature, change of pH, application of light, presence of ions, and the like. The gelation or solidification also may be achieved by cross-linking or using other covalent or non-covalent molecular interactions.

In still other embodiments, the formulation transforms from a solid-state associated with the microneedle to a dissolved state in the tissue. In such embodiments, the formulation may be administered to ocular tissue as a solid coating on the microneedle or encapsulated within the microneedle. In such embodiments, the formulation associated with the microneedle can include other excipients that serve various other functions. For example, the excipients may function to stabilize the drug (e.g., protect the drug from damage during the process of making the microneedles and/or storage of the microneedles and/or use of the microneedles), provide mechanical strength to the microneedle (e.g., providing sufficient strength so that the microneedle can be pressed into tissue without inappropriate deformation or damage), enhance wetting or facilitate solubilization of materials during manufacturing and use, and the like.

In some embodiments, the formulation controls the dissolution rate of the microneedles in whole or in part (e.g., of just the tip or base of the microneedle), for example, by the addition of highly water-soluble materials, including sugars. Preferentially increasing dissolution of the base of the microneedle may allow for the microneedle to be applied to a tissue, left in place for a short time during which the base of the microneedle at least partially dissolves, and then upon removing the device used to administer the microneedle, the microneedle would detach from that device and remain within the tissue.

Methods of Administration

Embodiments of the present description also include methods for administration of the above-described formulations to patients in need thereof. In particular, embodiments of methods are provided for non-surgical delivery of the above-described formulations to the eye of a patient, particularly for the treatment, diagnosis, or prevention of ocular disorders and maladies. Generally described, embodiments of methods for administering such formulations to an eye of a patient include inserting a microneedle into the eye at an insertion site and administering the formulation via the microneedle into the suprachoroidal space.

These methods enable targeted delivery of the drug to specific locations within the ocular tissue for treatment of ocular disorders and maladies, particularly posterior ocular disorders and choroidal maladies. Ocular tissues or locations to which or near to which it may be desirable to preferentially deliver the drug include the cornea, corneal epithelium, corneal stroma, corneal endothelium, limbus, corneal stroma adjacent to the limbus, sclera adjacent to the limbus, tear duct, lacrimal gland, eyelash, eyelid, sclera, conjunctiva, subconjunctival space, trabecular meshwork, Schlemm's canal, ciliary body, ciliary process, ciliary epithelium, ciliary stroma, aqueous humor, iris, lens, choroid, suprachoroidal space, retina, pars plana, macula, retina pigment epithelium, Bowman's membrane, subretinal space, optic nerve, vitreous humor, intravitreal space, periocular space, subTenon's space, tumors, sites of neovascularization, sites of trauma or injury, sites of infection, and cataracts. Other anatomical sites of the eye, as well as other sites of injury, disease, pathology, or otherwise needing treatment or alteration, are envisioned.

Targeted delivery using the formulations and methods provided herein is enabled at least in part due to the small size of the microneedles and ability to position the microneedles near specific tissues. In some embodiments, to target a specific tissue, the microneedle is positioned on the surface of the eye near the target tissue and then inserted to a controlled depth into the eye such that it reaches the tissue of interest. The depth of microneedle insertion can be controlled by the length of the microneedle, the force that is applied to the microneedle, the presence of additional device elements associated with the microneedle that controls its penetration depth, and by use of feedback mechanisms. In addition, the depth of insertion can be influenced by the thickness and mechanical properties of tissues in the path of the microneedle insertion. Specifically, deformation of the tissue can influence the depth of insertion, where tissue deformation can lead to less deep insertion if, for example, an indentation or dimple is formed on the surface of the tissue.

Feedback mechanisms that may be used to provide information about depth of insertion include one or more imaging techniques, such as ultrasound, optical coherence tomography, optical microscopy including fluorescence, confocal and other methods, and other imaging methods known in the art. These imaging techniques can also be used to provide information, such as tissue thickness, to guide subsequent microneedle use. Thus, feedback can be information obtained in advance of, during, or following insertion of the microneedle. Other forms of feedback can include electrical measurements, optical measurements, mechanical measurements, and the like. For example, as a microneedle passes through different tissues, the mechanical properties of the tissues may vary such that mechanical feedback about the microneedle's location with respect to the tissues can be obtained. Likewise, different tissues can have different electrical properties such that measurement of electrical properties can provide information about location in tissues.

In some embodiments, a volume (V) of a fluid formulation is administered through a hollow microneedle into the SCS of the eye at the insertion site. In other embodiments, the formulation is administered via a solid microneedle on which the formulation is coated or in which the formulation is otherwise associated. For example, in one embodiment, the solid microneedles is made out of a non-water-soluble material (e.g., a metal and/or a polymer) and the surface of the microneedle is coated with a formulation that contains the material to be delivered, the coating coming off the microneedle by dissolution or another mechanism after insertion. In another embodiment, the solid microneedle is made mostly or completely out of water-soluble materials, such that most or all the microneedle is released into the tissue after insertion.

In embodiments, it may be desirable for the formulation to remain substantially localized near the insertion site. For example, the spreading of the material can be minimized to remain within a targeted region. The spreading of the material may be characterized, for example, by the relative distance the formulation spreads from the insertion site and/or the volumetric spread of the formulation relative to the volume (V) of formulation infused via the microneedle or by dissolution from a solid microneedle. For example, in embodiments the spread of the majority of the drug and/or formulation from the insertion site may be less than 5 mm, 3 mm, 2 mm, 1 mm, 750 μm, 500 μm, 300 μm, 200 μm, or 100 μm, or the volumetric spread of the majority of the drug and/or formulation from the site of insertion site may be less than 20 times, 10 times, five times, three times, two times, or one time the cube root of the volume infused. By minimizing the spread of the formulation after administration, a majority of the drug and/or formulation may be preferentially located within the ocular tissue anterior to the equator, posterior to the equator, in the upper hemisphere, in the lower hemisphere, within one of the four quadrants of the eye (i.e., superior temporal, superior nasal, inferior temporal, inferior nasal) anterior to the equator, or within one of the four quadrants of the eye posterior to the equator.

In other embodiments, it may be advantageous for the spreading of the formulation to occur in two phases. Spreading may be limited or minimized over one period and more expansive over a second period. For example, in one embodiment, during the first period the fluid formulation is distributed over a first region which is less than about 10% of the SCS, and during the second period the fluid formulation is distributed over a second region which is greater than about 20% of the SCS, greater than about 50% of the SCS, or greater than about 75% of the SCS.

In some embodiments, the timescale during the first period corresponds to the infusion period (i.e., the time that the microneedle is in the tissue and fluid formulation is flowing out of the microneedle and into the tissue). Thus, the first period may be less than one hour, 30 minutes, 20 minutes, 15 minutes, 10 minutes, five minutes, three minutes, two minutes, one minute, 30 seconds, 10 seconds, or one second. For example, the first period may be from about 5 seconds to about 10 minutes.

In some embodiments, the timescale during the first period roughly corresponds to the time that the ocular tissue contains a significant portion of the liquid component of the formulation. Often, the liquid portion of the formulation will be cleared from the tissue relatively quickly, leaving behind the solid/dissolved components of the formulation in the tissue for longer period. For example, when injecting a formulation into the SCS, the formulation may include particles, a polymeric continuous phase in which the particles are dispersed, and a therapeutic agent which is in the particles and/or in the continuous phase. The polymeric continuous phase also may include various excipients. Upon injection into the SCS, all of these components of the formulation are introduced into the SCS and the SCS is expanded. Over a period, the polymeric continuous phase will be cleared out of the space, and the SCS will at least partially collapse. Thus, there is a limited opportunity to control migration of the drug, particles, and other materials within the SCS while it is expanded. It is during this time that at least initial spreading of the drug and/or formulation can occur. Conversely, it also may be advantageous to restrict movement of the drug and/or formulation while the suprachoroidal space is expanded. Thus, in embodiments, the first period may correspond to the entire period during which the suprachoroidal space remains expanded or a second period may correspond to the period during which the suprachoroidal space remains expanded after injection. In either case, this period may be for up to one hour, 30 minutes, 20 minutes, 15 minutes, 10 minutes, five minutes, three minutes, two minutes, one minute, 30 seconds, 10 seconds, or one second, depending on the amount of material injected and other factors.

In some embodiments, the method of administering the fluid formulation may be characterized by another time period which corresponds to the timescale after the fluid has substantially left the tissue, such as the SCS, such that the tissue is no longer significantly expanded (i.e., a second timescale after injection). In some embodiments, in which the first period includes both the timescale of injection and the timescale during which the SCS remains substantially expanded after injection, this time period may be referred to as the second period. This timescale may begin up to one hour, 30 minutes, 20 minutes, 15 minutes, 10 minutes, five minutes, three minutes, two minutes, one minute, 30 seconds, 10 seconds, one second after injection, depending on the amount of material injected and other factors. This timescale can continue for as long as the drug and/or formulation injected is present, needed or useful, which can be up to one hour, two hours, six hours, 12 hours, 24 hours, two days, three days, five days, seven days, 10 days, 14 days, three weeks, four weeks, one month, six weeks, two months, three months, four months, six months, or one year. For example, in embodiments this period may be from about 1 day to about 90 days.

In one embodiment, the method of administering the formulation includes some spreading during a first period, and then more spreading during a second period (i.e., the second timescale after injection). It is unexpected that there would be significant additional spreading during this second period when, for example, the SCS has collapsed and thereby limits movement. Indeed, if particles were injected into the SCS in unformulated water without any viscosifying agents, the converse would be true (i.e., there will be spreading during the first period, but very limited spreading during the second period). Thus, by properly formulating the formulation, spreading during the first period may be greater than, the same as, or less than that observed with unmodified water, but then there also can be significantly more spreading during the second period than that observed with unmodified water.

In embodiments, administration of these formulations may be characterized by the ratio of the distance of spreading from the site of injection during a later time period to the distance of spreading from the site of injection during the initial time period. For example, the ratio of the distance of spreading from the site of injection may be greater than 1, 1.25, 1.5, 1.75, 2.0, 2.5, 3.0, 4.0, or 5.0. The “later time period” may be up to one hour, two hours, six hours, 12 hours, 24 hours, two days, three days, five days, seven days, 10 days, 14 days, three weeks, four weeks, or one month after injection.

These methods enable delivery of a drug at one site for treatment using that drug at another site. For example, injection made at one site in the eye may be effective for treatment at another site in the eye. Thus, a drug may be administered into the SCS for treatment of glaucoma, for treatment in the ciliary body, for treatment in the trabecular meshwork, and/or for alteration of aqueous humor outflow by the conventional and/or unconventional pathways. For example a drug administered into the SCS anterior to the equator may be for treatment of a tissue posterior to the equator of the eye.

In some embodiments, the targeted administration of the formulation may be achieved by applying one or more external forces to direct movement of the formulation or its individual components after injection into the tissue. External forces that may be used to direct movement of the formulation or its individual components include gravitational, electromagnetic, centrifugal/centripetal, convective, ultrasonic, pressure or other forces. For example, a formulation can be injected into the SCS at one location and an external force can be used to keep the formulation or its individual components at that location, to spread it over a larger area within or outside the SCS, or to move it to a different location from the location where the injection occurred.

Such methods are preferably used with formulations including particles. For example, high density particles (e.g., having a specific gravity >1) may be injected into the eye with the cornea facing up. In this way, gravity acts to facilitate movement of the particles down, toward the back of the eye. Conversely, to move particles toward the front of the eye, the high-density particles may be injected into the eye with the cornea facing down such that gravity acts to facilitate movement of the particles down, toward the front of the eye. In still other embodiments, low density particles (e.g., having a specific gravity <1) may be injected into the eye with the cornea facing down. In this way, gravity acts to facilitate movement of the particles up, toward the back of the eye. Conversely, to move particles toward the front of the eye, the low-density particles may be injected into the eye with the cornea facing up such that gravity acts to facilitate movement of the particles up, toward the front of the eye.

Generally, particle movement within the SCS may be preferentially controlled by application of an external force while the SCS is open, before the tissue collapses back together again. For example, during and/or after an injection of the formulation into the SCS, the patient may be positioned appropriately in the gravitational field to promote movement of the particles to the desired location within the eye. After the injection, the patient may remain in the appropriate position in the gravitational field for a time sufficient for the SCS to collapse again (e.g., at least 30 seconds, one minute, two minutes, three minutes, five minutes, 10 minutes, 20 minutes, 30 minutes, one hour, or longer). The patient then may be permitted to move after that time because the tissue has collapsed to substantially close the SCS, thereby entrapping the particles. In this way, preferential movement of the particles within the tissue (e.g., suprachoroidal space) during the injection and the initial period after the injection may be controlled by the external force, and then may remain substantially localized or immobilized at the treatment site thereafter.

These methods enable substantial dose-sparing of drugs as compared to topical application of drugs, for example using eye drops. Dose-sparing refers to achieving a biological effect (e.g. reduction of intraocular pressure) using a lower dose. For example, a drug may be injected into a tissue adjacent to the ciliary body and/or trabecular meshwork, such as the SCS, preferably the anterior portion of the SCS, and achieve dose-sparing of a factor of 2, 5, 10, 20, 50, 100, 200, 500, 1000. This means that the dose administered is 2, 5, 10, 20, 50, 100, 200, 500, 1000 times lower than the one or more doses that are administered topically by eye drops to achieve the same or similar biological effect (i.e., a “comparative effective amount”). Dose-sparing is advantageous in that it enables extended therapy over longer times than could be achieved using prior art methods. Without dose-sparing, the dose needed for many weeks or months of therapy would be a very large dose. With dose-sparing, however, the dose needed for extended delivery would be significantly reduced.

The methods and formulations provided herein also advantageously permit preferential administration of formulations to or near targeted locations or tissues within the eye. When delivering a material to or near a specific location or tissue, the material can be preferentially delivered to that location with efficiency of approximately 100%, i.e. meaning that approximately 100% of the administered material is administered to the specific tissue or location. The material also can be delivered with an efficiency of at least 10%, more preferably at least 25%, more preferably at least 50%, more preferably at least 75%, more preferably at least 80%, more preferably at least 90%, more preferably at least 95%. For example, in embodiments in which the formulation includes particles, the particles may be delivered with efficiency effective to ensure at least 50%, at least 75%, at least 90%, or at least 95% of the particles are delivered to the treatment site.

These methods may be used to treat a wide range of ocular disorders and maladies in patients, including both adult and child human patients. Non-limiting examples of posterior ocular disorders amenable for treatment by the formulations and methods described herein include uveitis, glaucoma, macular edema, diabetic macular edema, retinopathy, age-related macular degeneration (for example, wet AMD or dry AMD), scleritis, optic nerve degeneration, geographic atrophy, choroidal disease, ocular sarcoidosis, optic neuritis, choroidal neovascularization, ocular cancer, genetic disease(s), autoimmune diseases affecting the posterior segment of the eye, retinitis (e.g., cytomegalovirus retinitis) and corneal ulcers. Such disorders may be acute or chronic. For example, the ocular disease may be acute or chronic uveitis. Acute uveitis occurs suddenly and may last for up to about six weeks, whereas with chronic uveitis the onset of signs and/or symptoms is gradual and the symptoms last longer than about six weeks. The ocular disorders may be caused by an infection from viruses, fungi, or parasites; the presence of noninfectious foreign substances in the eye; autoimmune diseases; or surgical or traumatic injury. Particular disorders caused by pathogenic organisms that can lead to uveitis or other types of ocular inflammation include, but are not limited to, toxoplasmosis, toxocariasis, histoplasmosis, herpes simplex or herpes zoster infection, tuberculosis, syphilis, sarcoidosis, Vogt-Koyanagi-Harada syndrome, Behcet's disease, idiopathic retinal vasculitis, Vogt-Koyanagi-Harada Syndrome, acute posterior multifocal placoid pigment epitheliopathy (APMPPE), presumed ocular histoplasmosis syndrome (POHS), birdsliot chroiclopathy, Multiple Sclerosis, sympathetic opthalmia, punctate inner choroidopathy, pars planitis, or iridocyclitis.

A variety of choroidal maladies are amenable for treatment by the formulations and methods described herein, including but not limited to, choroidal neovascularization, choroidal sclerosis, polypoidal choroidal vasculopathy, central sirrus choroidopathy, a multi-focal choroidopathy or a choroidal dystrophy. The choroidal dystrophy, for example, is central gyrate choroidal dystrophy, serpiginous choroidal dystrophy or total central choroidal atrophy. In some embodiments, a patient in need of treatment of a choroidal malady experiences subretinal exudation and bleeding, and the methods provided herein lessen the subretinal exudation and/or bleeding, compared to the subretinal exudation and/or bleeding experienced by the patient prior to administration of the drug formulation. In another embodiment, a patient in need of treatment experiences subretinal exudation and bleeding, and the subretinal exudation and bleeding experienced by the patient, after undergoing one of the non-surgical treatment methods provided herein, is less than the subretinal exudation and bleeding experienced by the patient after intravitreal therapy with the same drug at the same dose.

In an exemplary embodiment, the methods provide for administration of a drug formulation comprising an effective amount of an angiogenesis inhibitor to the SCS of an eye of a patient in need thereof. In one embodiment, the intraocular elimination half-life (t_{1/2}) of the angiogenesis inhibitor when administered to the SCS via the methods described herein is greater than the intraocular (t_{1/2}) of the angiogenesis inhibitor, when the identical dosage of the angiogenesis inhibitor is administered intravitreally, intracamerally, topically, parenterally or orally. In another embodiment, the mean intraocular maximum concentration (Cₘₐₓ) of the angiogenesis inhibitor when administered to the SCS via the methods described herein is greater than the intraocular maximum concentration of the angiogenesis inhibitor, when the identical dosage is administered intravitreally, intracamerally, topically, parenterally or orally. In another embodiment, the mean intraocular area under the curve (AUC₀₋ₜ) of the angiogenesis inhibitor when administered to the SCS via the methods described herein is greater than the intraocular AUCₒ₋₄ of the angiogenesis inhibitor, when the identical dosage of the angiogenesis inhibitor is administered intravitreally, intracamerally, topically, parenterally or orally.

In embodiments, the angiogenesis inhibitor may be interferon gamma 1β, interferon gamma 1β (Actimmune®) with pirfenidone, ACUHTR028, αVβ5, aminobenzoate potassium, amyloid P, ANG1122, ANG1170, ANG3062, ANG3281, ANG3298, ANG4011, anti-CTGF RNAi, Aplidin, astragalus membranaceus extract with salvia and schisandra chinensis, atherosclerotic plaque blocker, Azol, AZX100, BB3, connective tissue growth factor antibody, CT140, danazol, Esbriet, EXC001, EXC002, EXC003, EXC004, EXC005, F647, FG3019, Fibrocorin, Follistatin, FT011, a galectin-3 inhibitor, GKT137831, GMCT0I, GMCT02, GRMD01, GRMD02, GRN510, Heberon Alfa R, interferon-2β, ITMN520, JKB119, JKB121, JKB122, KRX168, LPA1 receptor antagonist, MGN4220, MIA2, microRNA 29a oligonucleotide, MMI0100, noscapine, PBI4050, PBI4419, PDGFR inhibitor, PF-06473871, PGN0052, Pirespa, Pirfenex, pirfenidone, plitidepsin, PRM151, Px102, PYN17, PYN22 with PYN17, Relivergen, rhPTX2 fusion protein, RXI109, secretin, STX100, TGF-β inhibitor, transforming growth factor, β-receptor 2 oligonucleotide, VA999260, or XV615.

Specific endogenous angiogenesis inhibitors may include endostatin, a 20 kDa C-terminal fragment derived from type XVIII collagen, angiostatin (a 38 kDa fragment of plasmin), or a member of the thrombospondin (TSP) family of proteins. In a further embodiment, the angiogenesis inhibitor is a TSP-1, TSP-2, TSP-3, TSP-4 and TSP-5. Other endogenous angiogenesis inhibitors may include a soluble VEGF receptor, e.g., soluble VEGFR-1 and neuropilin 1 (NPR1), angiopoietin-1, angiopoietin-2, vasostatin, calreticulin, platelet factor-4, a tissue inhibitor of metalloproteinase (TIMP) (e.g., TIMP 1, TIMP2, TIMP3, TIMP4), cartilage-derived angiogenesis inhibitor (e.g., peptide troponin I and chrondomodulin I), a disintegrin and metalloproteinase with thrombospondin motif 1, an interferon (IFN) (e.g., IFN-α, IFN-β, IFN-γ), a chemokine, (e.g., a chemokine having the C-X-C motif (e.g., CXCL10, also known as interferon gamma-induced protein 10 or small inducible cytokine B10)), an interleukin cytokine (e.g., IL-4, IL-12, IL-18), prothrombin, antithrombin III fragment, prolactin, the protein encoded by the TNFSFJ5 gene, osteopontin, maspin, canstatin, or proliferin-related protein.

In one embodiment, the angiogenesis inhibitor delivered via the methods described herein to treat a choroidal malady is an antibody. In a further embodiment, the antibody is a humanized monoclonal antibody. In even a further embodiment, the humanized monoclonal antibody is bevacizumab.

In one embodiment, the method is used to treat a choroidal malady. For example, the drug may be a nucleic acid administered to inhibit gene expression for treatment of the choroidal malady. The nucleic acid, in one embodiment, is a micro-ribonucleic acid (microRNA), a small interfering RNA (siRNA), a small hairpin RNA (shRNA), or a double stranded RNA (dsRNA), that targets a gene involved in angiogenesis. Thus, in one embodiment, the method to treat a choroidal malady comprises administering an RNA molecule to the suprachoroidal space of a patient in need thereof. The RNA molecule may be delivered to the suprachoroidal space via one of the microneedles described herein. For example, in one embodiment, the patient is being treated for PCV, and the RNA molecule targets HTRA1, CFH, elastin or ARMS2, such that the expression of the targeted gene is downregulated in the patient, upon administration of the RNA. In a further embodiment, the targeted gene is CFH, and the RNA molecule targets a polymorphism selected from rs3753394, rs800292, rs3753394, rs6680396, rs1410996, rs2284664, rs1329428, and rs1065489. In another embodiment, the patient is being treated for a choroidal dystrophy, and the RNA molecule targets the PRPH2 gene. In a further embodiment, the RNA molecule targets a mutation in the PRPH2 gene.

In one embodiment, the drug delivered to the SCS using the nonsurgical methods (e.g., microneedle devices and methods) herein is sirolimus (Rapamycin®, Rapamune®). In one embodiment, the non-surgical drug delivery methods are used in conjunction with rapamycin to treat, prevent and/or ameliorate a wide range of diseases or disorders including, but not limited to: abdominal neoplasms, acquired immunodeficiency syndrome, acute coronary syndrome, acute lymphoblastic leukemia, acute myelocytic leukemia, acute non-lymphoblastic leukemia, adenocarcinoma, adenoma, adenomyoepithelioma, adnexal diseases, anaplastic astrocytoma, anaplastic large cell lymphoma, anaplastic plasmacytoma, anemia, angina pectoris, angioimmunoblastic lymphadenopathy with dysproteinemia, angiomyolipoma, arterial occlusive diseases, arteriosclerosis, astrocytoma, atherosclerosis, autoimmune diseases, B-cell lymphomas, blood coagulation disorders, blood protein disorders, bone cancer, bone marrow diseases, brain diseases, brain neoplasms, breast neoplasms, bronchial neoplasms, carcinoid syndrome, carcinoid tumor, carcinoma, squamous cell carcinoma, central nervous system diseases, central nervous system neoplasms, choroid diseases, choroid plexus neoplasms, choroidal neovascularization, choroiditis, chronic lymphocytic leukemia, chronic myeloid leukemia, chronic myelomonocytic leukemia, chronic myeloproliferative disorders, chronic neutrophilic leukemia, clear cell renal cell carcinoma, colonic diseases, colonic neoplasms, colorectal neoplasms, coronary artery disease, coronary disease, coronary occlusion, coronary restenosis, coronary stenosis, coronary thrombosis, cutaneous T-cell lymphoma, diabetes mellitus, digestive system neoplasms, dry eye syndromes, ear diseases, edema, endocrine gland neoplasms, endocrine system diseases, endometrial neoplasms, Endometrial stromal tumors, Ewing's sarcoma, exanthema, eye neoplasms, fibrosis, follicular lymphoma, gastrointestinal diseases, gastrointestinal neoplasms, genital neoplasms, glioblastoma, glioma, gliosarcoma, graft vs host disease, hematologic diseases, hematologic neoplasms, hemorrhagic disorders, hemostatic disorders, Hodgkin disease, Hodgkin lymphoma, homologous wasting disease, immunoblastic lymphadenopathy, immunologic deficiency syndromes, immunoproliferative disorders, infarction, inflammation, intestinal diseases, intestinal neoplasms, ischemia, kidney cancer, kidney diseases, kidney neoplasms, leukemia, B-Cell, leukemia, lymphoid, liver cancer, liver diseases, lung diseases, lymphatic diseases, lymphoblastic lymphoma, lymphoma, macular degeneration, macular edema, melanoma, mouth neoplasms, multiple myeloma, myelodysplastic syndromes, myelofibrosis, myeloproliferative disorders, neuroectodermal tumors, neuroendocrine tumors, neuroepithelioma, neurofibroma, renal cancer, respiratory tract diseases, retinal degeneration, retinal diseases, retinal neoplasms, retinoblastoma, rhabdomyosarcoma, thoracic neoplasms, uveitis, vascular diseases, Waldenstrom Macroglobulinemia, and wet macular degeneration. In addition, delivery of rapamycin using the microneedle devices and methods disclosed herein may be combined with one or more agents listed herein or with other agents known in the art.

In one embodiment, the VEGF antagonist delivered via the non-surgical methods described herein is an antagonist of a VEGF receptor (VEGFR), i.e., a drug that inhibits, reduces, or modulates the signaling and/or activity of a VEGFR. The VEGFR may be a membrane-bound or soluble VEGFR. In a further embodiment, the VEGFR is VEGFR-1, VEGFR-2 or VEGFR-3. In one embodiment, the VEGF antagonist targets the VEGF-C protein. In another embodiment, the VEGF modulator is an antagonist of a tyrosine kinase or a tyrosine kinase receptor. In another embodiment, the VEGF modulator is a modulator of the VEGF-A protein. In yet another embodiment, the VEGF antagonist is a monoclonal antibody. In a further embodiment, the monoclonal antibody is a humanized monoclonal antibody.

In one embodiment, the drug formulation delivered to the SCS of an eye of a patient in need thereof via the methods described herein comprises an effective amount of vascular permeability inhibitor. In one embodiment, the vascular permeability inhibitor is a vascular endothelial growth factor (VEGF) antagonist or an angiotensin converting enzyme (ACE) inhibitor. In a further embodiment, the vascular permeability inhibitor is an angiotensin converting enzyme (ACE) inhibitor and the ACE inhibitor is captopril.

In one embodiment, the drug formulation delivered to the SCS of an eye of a patient in need thereof via the methods described herein comprises a steroidal compound, which may include hydrocortisone, hydrocortisone-17-butyrate, hydrocortisone-17-aceponate, hydrocortisone-17-buteprate, cortisone, tixocortol pivalate, prednisolone, methylprednisolone, prednisone, triamcinolone, triamcinolone acetonide, mometasone, amcinonide, budesonide, desonide, fluocinonide, halcinonide, bethamethasone, bethamethasone dipropionate, dexamethasone, fluocortolone, hydrocortisone-17-valerate, halometasone, alclometasone dipropionate, prednicarbate, clobetasone-17-butyrate, clobetasol-17-propionate, fluocortolone caproate, fluocortolone pivalate, fluprednidene acetate or prednicarbate.

In one embodiment, the drug formulation delivered is a specific class of NSAID, non-limiting examples of which include salicylates, propionic acid derivatives, acetic acid derivatives, enolic acid derivatives, fenamic acid derivatives and cyclooxygenase-2 (COX-2) inhibitors. In one embodiment, one or more of the following NSAIDs are provided in the drug formulation: acetylsalicylic acid, diflunisal, salsalate, ibuprofen, dexibuprofen, naproxen, fenoprofen, keotoprofen, dexketoprofen, flurbiprofen, oxaprozin, loxaprofen, indomethacin, tolmetin, sulindac, etodolac, ketorolac, diclofenac or nabumetone, piroxicam, meloxicam, tenoxicam, droxicam, lornoxicara or isoxicam, mefanamic acid, meclofenamic acid, flufenamic acid, tolfenamic acid, celecoxib, refecoxib, valdecoxib, parecoxib, lumiracoxib, etoricoxib, or firocoxib.

Other examples of anti-inflammatory drugs, that can be used to treat a posterior ocular disorder or a choroidal malady, choroidal neovascularization, or subretinal exudation, include, but are not limited to: mycophenoiate, remicase, nepafenac, 19AV agonist(s), 19GJ agonists, 2MD analogs, 4SC101, 4SC102, 57-57, 5-HT2 receptor antagonist, 64G12, A804598, A967079, AAD2004, AB1010, AB224050, abatacept, etaracizumab (Abegrin™), Abevac®, AbGn134, AbGn168, Abki, ABN912, ABR215062, ABR224050, cyclosporine (Abrammune®), docosanol (behenyl alcohol, Abreva®), ABS15, ABS4, ABS6, ABT122, ABT325, ABT494, ABT874, ABT963, ABXIL8, ABXRB2, AC430, Accenetra, lysozyme chloride (Acdeam®), ACE772, aceclofenac (Acebloc, Acebid, Acenac), acetaminophen, chlorzoxazone, serrapeptase, tizanidine hydrochloride, betadex, Aceclogesic Plus, Aceclon, Acecloren, Aceclorism, acecrona, Aceffein, acemetacin, asprin (Acenterine), Acetal-SP (Aceclofenac-combination), Acetyl-G, acetylsalicylate dl-lysine, acetylsalicylic acid, Acicot, Acifine, Acik, Aclocen, Acloflam-P, Aclomore, Aclon, A-CQ, ACS15, actarit, Actemra, Acthelea liofilizado, Actifast, Actimab-B, Actiquim, Actirin, Actis PLUS, activated leukocyte cell adhesion molecule antibody, Acular X, AD452, adalimumab, ADAMTSS inhibitor, ADC1001, Adco-Diclofenac, Adco-Indomethacin, Adco-Meloxicam, Adco-Naproxen, Adco-Piroxicam, Adcort, Adco-Sulindac, adenosine triphosphate disodium, AdenosineA2a Receptor Agonist, Adimod, Adinos, Adioct, Adiodol, Adipoplus, adipose derived stem and/or regenerative cells, Adizen, Adpep, Advacan, Advagraf, Advel, Adwiflam, AEB071, Aental, Afenac, Affen Plus, Afiancen, Afinitor, Aflamin, Aflazacort, Aflogen, Afloxan, AFM15, AFM16, AFM17, AFM23, Afpred-Dexa, AFX200, AG011, Agafen, aganirsen, AGI1096, Agidex, AGS010, Agudol, A-Hydrocort, AIK1, AIN457, Airtal, AIT110, AJM300, ajulemic acid, AK106, AL-24-2A1, AL4-1A1, Ala Cort, Alanz, Albumin immune-globulin, alclometasone dipropionate, ALD518, aldesleukin, Aldoderma, alefacept, alemtuzmab, Alequel™, Alergolon, Alergosone, Aletraxon, Alfenac, Algason, Algin vek coat, Algioflex, Algirex, Aigivin Plus, alicaforsen sodium, Alin, Alinia, Aliviodol, Aliviosin, alkaline phosphatase, ALKS6931, allantoin, Allbupen, Allmol, Allochrysine, allogeneic endothelial cells, allogeneic mesenchymal precursor cells, allogeneic mesenchymal stem cells, alminoprofen, alpha 1 antitrypsin, Alpha 7 nicotinic agonists, alpha amylase, alpha chymotrypsin, alpha fetoprotein, alpha linolenic acid, alpha-1-antitrypsin, α2β1 integrin inhibitors, Alphacort, Alphafen, alpha-hexidine, alpha-trypsin, Alphintern, Alpinamed mobility omega 3, Alpoxen, AL-Revl, Alterase, ALX0061, ALX0761, ALXN1007, ALXN1102, AM3840, AM3876, AMAB, AMAP102, Amason, Ambene, AmbezimG, amcinonide, AME133v, Amecin, Ameloteks, A-Methapred, Amevive, AMG108, AMG139, AMG162, AMG181, AMG191, AMG220, AMG623, AMG674, AMG714, AMG719, AMG729, AMG827, Amidol, amifampridine phosphate, diclofenac (Emifenac®), Amimethacin, amiprilose hydrochloride, Amiprofen, Ammophos, Amoflam, AMP 110, Ampikyy, Ampion, ampiroxicam, amtolmetin guacil, AMX256, AN6415, ANA004, ANA506, Anabu, Anacen, Anaflam, Anaflex ACI, Anaida, anakinra, Analgen Artritis, Anapan, Anaprox, Anavan, Anax, Anco, andrographis, Ancol, Anergix, Anervax.RA™ (therapeutic peptide vaccine), Anflene, ANG797, Anilixin, Anmerushin, Annexin 1 peptides, annexin A5, Anodyne, Ansaid, Anspirin, Antarene, anti BST2 antibody, anti C5a MAb, anti ILT7 antibody, anti VLA1 antibody, anti-alphal 1 antibody, anti-CD4 802-2, anti-CD86 monoclonal antibody, anti-chemokine, anti-DC-SIGN, anti-HMGB-1 MAb, anti-IL-18 Mab, anti-IL-1R MAb, anti-IL-1R MAb, anti-IL23 BRISTOL, anti-interleukin-1β antibody, anti-LIGHT antibody, anti-MIF antibody, anti-miR181a, antioxidant inflammation modulators, Antiphlamine, AntiRAGE MAb, antithrombin III, Anti-TIRC-7 MAb, Anusol-HC, Anyfen, AP105, AP1089, AP1189, AP401, AP501, apazone, APD334, Apentac, APG103, Apidone, apilimod mesylate, Apitac, Apitoxin, Apizel, APN inhibitor, apo-azathioprine, Apo-dexamethasone, ApoE mimetics, ApoFasL, apo-Indomethacin, apo-mefenamic, apo-methotrexate, apo-nabumetone, Apo-Napro-NA, apo-Naproxen, aponidin, apo-Phenylbutazone, apo-Piroxicam, apo-Sulin, Apo-Tenoxicam, apo-Tiaprofenic, Apranax, apremilast, apricoxib, Aprofen, Aprose, Aproxen, APX001 antibody, APX007 antibody, APY0201, AqvoDex, AQX108, AQX1125, AQX131135, AQX140, AQX150, AQX200, AQX356, AQXMN100, AQXMN106, ARA290, Arava, Arcalyst, Arcoxia, Arechin, Arflur, ARG098, ARG301, arginine aescin, arginine deiminase (pegylated), ARGX109 antibody, ARGX110, Arheuma, Aristocort, Aristospan, Ark-AP, ARN4026, Arofen, Aroff EZ, Arolef, Arotal, Arpibru, Arpimune, Arpu Shuangxin, ARQ101, Arrestin SP, Arrox, ARRY162, ARRY371797, ARRY614, ARRY872, ART621, Artamin, Arthfree, Artho Tech, Arthrexin, Arthrispray, Arthrotec, aeterna shark cartilage extract (Arthrovas™, Neoretna™, Psovascar™), Artifit, Artigo, Artin, Artinor, Artisid, Artoflex, Artren Hipergel, Artridol, Artrilase, Artrocaptin, Artrodiet, Artrofen, Artropan, Artrosil, Artrosilene, Artrotin, Artrox, Artyflam, Arzerra, AS604850, AS605858, Asacol, ASA-Grindeks, Asazipam, Aseclo, ASF1096, ASK8007, ASKP1240, ASLAN003, Asmo ID, Asonep, ASP015K, ASP2408, ASP2409, Aspagin, Aspeol, Aspicam, Aspirimex, AST120, astaxanthin, AstroCort, Aszes, AT002 antibody, AT007, AT008 antibody, AT010, AT1001, atacicept, Ataspin, Atepadene, Atgam, ATG-Fresenius, Athrofen, ATI003, atiprimod, ATL1222, ATN103, ATN192, ATR107, Atri, Atrmin, Atrosab antibody, ATX3105, AU801, auranofin, Aurobin, Auropan, Aurothio, aurotioprol, autologous adipose derived regenerative cells, Autonec, Avandia, AVE9897, AVE9940, Avelox, Avent, AVI3378, Avloquin, AVP13546, AVP13748, AVP28225, AVX002, Axcel Diclofenac, Axcel Papain, Axen, AZ17, AZ175, Azacortid, AZA-DR, Azafrine, Azamun, Azanin, Azap, Azapin, Azapren, Azaprin, Azaram, Azasan, azathioprine, AZD0275, AZD0902, AZD2315, AZD5672, AZD6703, AZD7140, AZD8309, AZD8566, AZD9056, Azet, Azintrel, azithromycin, Az-od, Azofit, Azolid, Azoran, Azulene, Azulfidine, Azulfin, Bl antagonists, Baclonet, BAF312, BAFF Inhibitor, Bages, Baily S.P., Baleston, Balsolone, baminercept alfa, bardoxolone methyl, baricitinib, Barotase, Basecam, basiliximab, Baxmune, Baxo, BAY869766, BB2827, BCX34, BCX4208, Becfine, Beclate-C, Beclate-N, Beclolab Q, beclomethasone dipropionate, Beclorhin, Becmet-CG, Begita, Begti, belatacept, belimumab, Belosalic, Bemetson, Ben, Benevat, Benexam, Benflogin, Benisan, Benlysta, benorilate, Benoson, benoxaprofen, Bentol, benzydamine hydrochloride, Benzymin, Beofenac, Berafen, Berinert, Berlofen, Bertanel, Bestamine, Bestofen, Beta Nicip, Betacort, Betacorten G, Betafoam, beta-glucan, Betalar, Beta-M, Betamed, Betamesol, betamethasone, betamethasone dipropionate, betamethasone sodium, betamethasone sodium phosphate, betamethasone valerate, Betane, Betanex, Betapanthen, Betapar, Betapred, Betason, Betasonate, Betasone, Betatrinta, Betaval, Betazon, Betazone, Betesil, Betnecort, Betnesol, Betnovate, Bextra, BFPC13, BFPC18, BFPC21, BFPT6864, BG12, BG9924, BI695500, BI695501, BIA12, Big-Joint-D, BIIB023 antibody, Bi-ksikam, Bingo, BioBee, Bio-Cartilage, Bio-C-Sinkki, Biodexone, Biofenac, Bioreucarn, Biosone, Biosporin, BIRB796, Bitnoval, Bitvio, Bivigam, BKT140, BKTP46, BL2030, BL3030, BL4020, BL6040, BL7060, BL11300, blisibimod, Blokium B12, Blokium Gesic, Blokium, BMS066, BMS345541, BMS470539, BMS561392, BMS566419, BMS582949, BMS587101, BMS17399, BMS936557, BMS945429, BMS-A, BN006, BN007, BNP166, Bonacort, Bonas, bone marrow stromal cell antigen 2 antibody, Bonflex, Bonifen, Boomiq, Borbit, Bosong, BR02001, BR3-FC, Bradykinin B1 Receptor Antagonist, Bredinin, Brexecam, Brexin, Brexodin, briakinumab, Brimani, briobacept, Bristaflam, Britten, Broben, brodalumab, Broen-C, bromelains, Bromelin, Bronax, Bropain, Brosiral, Bruace, Brufadol, Brufen, Brugel, Brukil, Brusil, BT061, BT19, BT kinase inhibitors, BTT1023 antibody, BTT1507, bucillamine, Bucillate, Buco Reigis, bucolome, Budenofalk, budesonide, Budex, Bufect, Bufencon, Bukwang Ketoprofen, Bunide, Bunofen, Busilvex, busulfan, Busulfex, Busulipo, Butartrol, Butarut B12, Butasona, Butazolidin, Butesone, Butidiona, BVX10, BXL628, BYM338, B-Zone, C1 esterase inhibitor, C243, c4462, c5997, CSaQb, c7198, c9101, C9709, c9787, CAB101, cadherin 11 antibody, caerulomycin A, CAL263, Calcort, Calmatel, CAM3001, Camelid Antibodies, Camlox, Camola, Campath, Camrox, Camtenam, canakinumab, candida albicans antigen, Candin, cannabidiol, CAP 1.1, CAP1.2, CAP2.1, CAP2.2, CAP3.1, CAP3.2, Careram, Carimune, Cariodent, Cartifix, CartiJoint, Cartilago, Cartisafe-DN, Cartishine, Cartivit, Cartril-S, Carudol, CaspaCIDe, Casyn, CAT1004, CAT1902, CAT2200, Cataflam, Cathepsin S inhibitor, Catlep, CB0114, CB2 agonist CC0478765, CC10004, CC10015, CC1088, CC11050, CC13097, CC15965, CC16057, CC220, CC292, CC401, CC5048, CC509, CC7085, CC930, CCR1 antagonist, CCR6 inhibitor, CCR7 antagonist, CCRL2 antagonist, CCX025, CCX354, CCX634, CD Diclofenac, CD102, CD103 antibody, CD137 antibody, CD16 antibody, CD18 antibody, CD19 antibody, CD1d antibody, CD20 antibody, CD200Fc, CD209 antibody, CD24, CD3 antibody, CD30 antibody, CD32A antibody, CD32B antibody, CD4 antibody, CD40 ligand, CD44 antibody, CD64 antibody, CDC839, CDC998, CDIM4, CDIM9, CD 9-Inhibitor, CDP146, CDP323, CDP484, CDP6038, CDP870, CDX1135, CDX301, CE224535, Ceanel, Cebedex, Cebutid, Ceclonac, Ceex, CEL2000, Celact, Celbexx, Celcox, Celebiox, Celebrex, Celebrin, Celecox, celecoxib, Celedol, Celestone, Celevex, Celex, CELG4, Cell adhesion molecule antagonists, CellCept, Cellmune, Celosti, Celoxib, Celprot, Celudex, cenicriviroc mesylate, cenplacel-1, CEP11004, CEP37247, CEP37248, Cephyr, Ceprofen, Certican, certolizumab pegol, Cetofenid, Cetoprofeno, cetylpyridimum chloride, CF10I, CF402, CF502, CG57008, CGEN15001, CGEN15021, CGEN 15051, CGEN15091, CGEN25017, CGEN25068, CGEN40, CGEN54, CGEN768, CGEN855, CGI1746, CGI560, CGI676, Cgtx-Peptides, CHI504, CH4051, CH4446, chaperonin 10, chemokine C-C motif ligand 2, chemokine C-C motif ligand 2 antibody, chemokine C-C motif ligand 5 antibody, chemokine C-C motif receptor 2 antibody, chemokine C-C motif receptor 4 antibody, chemokine C-X-C motif ligand 10 antibody, chemokine C-X-C motif ligand 12 aptamer, Chemotaxis Inhibitor, Chillmetacin, chitinase 3-like 1, Chlocodemin, Chloquin, chlorhexidine gluconate, chloroquine phosphate, choline magnesium trisalicylate, chondroitin sulfate, Chondroscart, CHR3620, CHR4432, CHR5154, Chrysalin, Chuanxinlian, Chymapra, Chymotase, chymotrypsin, Chytmutrip, CI202, CI302, Cicloderm-C, Ciclopren, Cicporal, Cilamin, Cimzia, cinchophen, cinmetacin, cinnoxicam, Cinoderm, Cinolone-S, Cinryze, Cipcorlin, cipemastat, Cipol-N, Cipridanol, Cipzen, Citax F, Citogan, Citoken T, Civamide, CJ042794, CJ14877, c-Kit monoclonal antibody, cladribine, Clafen, Clanza, Ciaversal, clazakizumab, Clearoid, Clease, Clevegen, Clevian, Clidol, Clindac, Clinoril, Cliptol, Clobenate, Clobequad, clobetasol butyrate, clobetasol propionate, Clodol, clofarabine, Clofen, Clofenal LP, Clolar, Clonac, Clongamma, clonixin lysine, Clotasoce, Clovacort, Clovana, Cloxin, CLT001, CLT008, C-MAF Inhibitor, CMPXIO23, Cnac, CNDO201, CNI1493, CNTO136, CNT0148, CNTO1959, Cobefen, CoBenCoDerm, Cobix, Cofenac, COG241, COL179, colchicine, Colchicum Dispert, Colchimax, Colcibra, Coledes A, Colesol, Coiifoam, Colirest, collagen, type V, Comcort, complement component (3b/4b) receptor 1, complement component C1s inhibitors, complement component C3, complement factor 5a receptor antibody, complement factor D antibody, Condrosulf, Condrotec, Condrothin, conestat alfa, connective tissue growth factor antibody, Coolpan, Copaxone, Copiron, Cordefla, Corhydron, Cort S, Cortan, Cortate, Cort-Dome, Cortecetine, Cortef, Corteroid, Corticap, Corticas, Cortic-DS, corticotropin, Cortiderm, Cortidex, Cortiflam, Cortinet M, Cortinil, Cortipyren B, Cortiran, Cortis, Cortisolu, cortisone acetate, Cortival, Cortone acetate, Cortopin, Cortoral, Cortril, Cortypiren, Cosamine, Cosone, cosyntropin, COT Kinase Inhibitor, Cotilam, Cotrisone, Cotson, Covox, Cox B, COX-2/5-LO Inhibitors, Coxeton, Coxflam, Coxicam, Coxitor, Coxtral, Coxypar, CP195543, CP412245, CP424174, CP461, CP629933, CP690550, CP751871, CPSI2364, C-quin, CR039, CR074, CR106, CRA102, CRAC channel inhibitor, CRACM ion channel inhibitor, Cratisone, CRB15, CRC4273, CRC4342, C-reactive protein 2-methoxyethyl phosphorothioate oligonucleotide, CreaVax-RA, CRH modulators, critic-aid, Crocam, Crohnsvax, Cromoglycic acid, cromolyn sodium, Cronocorteroid, Cronodicasone, CRTX803, CRx119, CRx139, CRx150, CS502, CS670, CS706, CSFIR Kinase inhibitors, CSL324, CSL718, CSL742, CT112, CT1501R, CT200, CT2008, CT2009, CT3, CT335, CT340, CT5357, CT637, CTP05, CTP10, CT-P13, CTP17, Cuprenil, Cuprimine, Cuprindo, Cupripen, Curaquin, Cutfen, CWF0808, CWP271, CX1020, CX1030, CX1040, CX5011, Cx611, Cx621, Cx911, CXC chemokine receptor 4 antibody, CXCL13 antibodies, CXCR3 antagonists, CXCR4 antagonist, Cyathus 1104 B, Cyclo-2, Cyclocort, cyclooxygenase-2 inhibitor, cyclophosphamide, Cyclorine, Cyclosporin A Prodrug, Cyclosporin analogue A, cyclosporine, Cyrevia, Cyrin CLARIS, CYT007TNFQb, CYT013ILlbQb, CYT015IL17Qb, CYTO2OTNFQb, CYT107, CYT387, CYT99007, cytokine inhibitors, Cytopan, Cytoreg, CZC24832, D1927, D942IC, daclizumab, danazol, Danilase, Dantes, Danzen, dapsone, Dase-D, Daypro, Daypro Alta, Dayrun, Dazen, DB295, DBTP2, D-Cort, DD1, DD3, DE096, DE098, Debio0406, Debio0512, Debio0615, Debio0618, Debio1036, Decaderm, Decadrale, Decadron, Decadronal, Decalon, Decan, Decason, Decdan, Decilone, Declophen, Decopen, Decorex, Decorten, Dedema, Dedron, Deexa, Defcort, De-flam, Deflamat, Defian, Deflanil, Deflaren, Deflaz, deflazacort, Defnac, Defnalone, Defnil, Defosalic, Defsure, Defza, Dehydrocortison, Dekort, Delagil delcasertib, delmitide, Delphicort, Deltacorsolone prednisolone (Deltacortril), Deltafluorene, Deltasolone, Deltasone, Deltastab, Deltonin, Demarin, Demisone, Denebola, denileukin diftitox, denosumab, Denzo, Depocortin, Depo-medrol, Depomethotrexate, Depopred, Deposet, Depyrin, Derinase, Dermol, Dermolar, Dermonate, Dermosone, Dersone, Desketo, desonide, desoxycorticosterone acetate, Deswon, Dexa, Dexabene, Dexacip, Dexacort, dexacortisone, Dexacotisil, dexadic, dexadrin, Dexadron, Dexafar, Dexahil, Dexalab, Dexalaf, Dexalet, Dexalgen, dexallion, dexalocal, Dexalone, Dexa-M, Dexamecortin, Dexamed, Dexamedis, dexameral, Dexameta, dexamethasone, dexamethasone acetate, dexamethasone palmitate, dexamethasone phosphate, dexamethasone sodium metasulfobenzoate, dexamethasone sodium phosphate, Dexamine, Dexapanthen, Dexa-S, Dexason, Dexatab, Dexatopic, Dexaval, Dexaven, Dexazolidin, Dexazona, Dexazone, Dexcor, Dexibu, dexibuprofen, Dexico, Dexifen, Deximune, dexketoprofen, dexketoprofen trometamol, Dexmark, Dexomet, Dexon I, Dexonalin, Dexonex, Dexony, Dexoptifen, Dexpin, Dextan-Plus, dextran sulfate, Dezacor, Dfz, diacerein, Diannexin, Diastone, Dicarol, Dicasone, Dicknol, Diclo, Diclobon, Diclobonse, Diclobonzox, Diclofast, Diclofen, diclofenac, diclofenac beta-dimethylaminoethanol, diclofenac deanol, diclofenac diethylamine, diclofenac epolamine, diclofenac potassium, diclofenac resinate, diclofenac sodium, Diclogen AGIO, Diclogen Plus, Diclokim, Diclomed, Diclo-NA, Diclonac, Dicloramin, Dicloran, Dicloreum, Diclorism, Diclotec, Diclovit, Diclowal, Diclozem, Dico P, Dicofen, Dicoliv, Dicorsone, Dicron, Dicser, Difena, Diffutab, diflunisal, dilmapimod, Dilora, dimethyl sulfone, Dinac, D-Indomethacin, Dioxaflex Protect, Dipagesic, Dipenopen, Dipexin, Dipro AS, Diprobeta, Diprobetasone, Diproklenat, Dipromet, Dipronova, Diprosone, Diprovate, Diproxen, Disarmin, Diser, Disopain, Dispain, Dispercam, Distamine, Dizox, DLT303, DLT404, DM199, DM99, DMI9523, dnaJP1, DNX02070, DNX04042, DNX2000, DNX4000, docosanol, Docz-6, Dolamide, Doclaren, Dolchis, Dolex, Dolflam, Dolfre, Dolgit, Dolmax, Dolmina, Dolo Ketazon, Dolobest, Dolobid, Doloc, Dolocam, Dolocartigen, Dolofit, Dolokind, Dolomed, Dolonac, Dolonex, Dolotren, Dolozen, Dolquine, Dom0100, Dom0400, Dom0800, Domet, Dometon, Dominadol, Dongipap, Donica, Dontisanin, doramapimod, Dorixina Relax, Dormelox, Dorzine Plus, Doxatar, Doxtran, DP NEC, DP4577, DP50, DP6221, D-Penamine, DPIV/APN Inhibitors, DR1 Inhibitors, DR4 Inhibitors, DRA161, DRA162, Drenex, DRF4848, DRL15725, Drossadin, DSP, Duexis, Duo-Decadron, Duoflex, Duonase, DV1079, DV1179, DWJ425, DWP422, Dymol, DYN15, Dynapar, Dysmen, E5090, E6070, Easy Dayz, Ebetrexat, EBI007, ECO286, ECO565, EC0746, Ecax, echinacea purpurea extract, EC-Naprosyn, Econac, Ecosprin 300, Ecridoxan, eculizumab, Edecam, efalizumab, Efcortesol, Effigel, Eflagen, Efridol, EGFR Antibody, EGS21, eIF5A1 siRNA, Ekarzin, elafin, Eldoflam, Elidel, Eliflam, Elisone, Elmes, Elmetacin, ELND001, ELND004, elocalcitol, Elocom, elsibucol, Emanzen, Emcort, Emifen, Emifenac, emorfazone, Empynase, emricasan, Emtor, Enable, Enbrel, Enceid, EncorStat, Encortolon, Encorton, Endase, Endogesic, Endoxan, Enkorten, Ensera, Entocort, Enzylan, Epanova, Eparang, Epatec, Epicotil, epidermal growth factor receptor 2 antibody, epidermal growth factor receptor antibody, Epidixone, Epidron, Epiklin, EPPA1, epratuzumab, EquiO, Erac, Erazon, ERB041, ERB196, Erdon, EryDex, escherichia coli enterotoxin B subunit, Escin, E-Selectin Antagonists, Esfenac, ESN603, esonarimod, Esprofen, estetrol, Estopein, Estrogen Receptor beta agonist, etanercept, etaracizumab, ETC001, ethanol propolis extract, ETI511, etiprednol dicloacetate, Etodin, Etodine, Etodol, etodolac, Etody, etofenamate, Etol Fort, Etolac, Etopin, etoricoxib, Etorix, Etosafe, Etova, Etozox, Etura, Eucob, Eufans, eukaryotic translation initiation factor 5A oligonucleotide, Eunac, Eurocox, Eurogesic, everolimus, Evinopon, EVT401, Exaflam, EXEL9953, Exicort, Expen, Extra Feverlet, Extrapan, Extrauma, Exudase, F16, F991, Falcam, Falcol, Falzy, Farbovil, Farcomethacin, Farnerate, Farnezone, Farotrin, fas antibody, Fastflam, FasTRACK, Fastum, Fauldmetro, FcgammaRIA antibody, FE301, Febrofen, Febrofid, felbinac, Feldene, Feldex, Feloran, Felxicam, Fenac, Fenacop, Fenadol, Fenaflan, Fenarnic, Fenaren, Fenaton, Fenbid, fenbufen, Fengshi Gutong, Fenicort, Fenopine, fenoprofen calcium, Fenopron, Fenris, Fensupp, Fenxicam, fepradinol, Ferovisc, Feverlet, fezakinumab, FG3019, FHT401, FHTCT4, FID114657, figitumumab, Filexi, filgrastim, Fillase, Final, Findoxin, fingolimod hydrochloride, firategrast, Firdapse, Fisiodar, Fivasa, FK778, Flacoxto, Fladalgin, Flagon, Flamar, Flamcid, Flamfort, Flamide, Flaminase, Flamirex Gesic, Flanid, Flanzen, Flaren, Flash Act, Flavonoid Anti-inflammatory Molecule, Flebogamma DIF, Flenac, Flex, Flexafen 400, Flexi, Flexidol, Flexium, Flexon, Flexono, Flogene, Flogiatrin B12, Flogomin, Flogoral, Flogosan, Flogoter, Flo-Pred, Flosteron, Flotrip Forte, Flt3 inhibitors, fluasterone, Flucam, Flucinar, fludrocortisone acetate, flufenamate aluminum, flumethasone, Flumidon, flunixin, fluocinolone, fluocinolone acetonide, fluocinonide, fluocortolone, Fluonid, fluorometholone, Flur, flurbiprofen, Fluribec, Flurometholone, Flutal, fluticasone, fluticasone propionate, Flutizone, Fluzone, FM101 antibody, fms-related tyrosine kinase 1 antibody, Folitrax, fontolizumab, formic acid, Fortecortin, Fospeg, fostamatinib disodium, FP1069, FP13XX, FPA008, FPA031, FPT025, FR104, FR167653, Framebin, Frime, Froben, Frolix, FROUNT Inhibitors, Fubifen PAP, Fucole ibuprofen, Fulamotol, Fulpen, Fungifin, Furotalgin, fusidate sodium, FX002, FX141L, FX201, FX300, FX87L, Galectin modulators, gallium maltolate, Gamimune N, Gammagard, Gamma-I.V., GammaQuin, Gamma-Venin, Gamunex, Garzen, Gaspirin, Gattex, GBR500, GBR500 antibody, GBT009, G-CSF, GED0301, GED0414, Gefenec, Gelofen, Genepril, Gengraf, Genimune, Geniquin, Genotropin, Genz29155, Gerbin, gevokizumab, GF01564600, Gilenia, Gilenya, givinostat, GL0050, GL2045, glatiramer acetate, Globulin, Glortho Forte, Glovalox, Glovenin-I, GLPG0259, GLPG0555, GLPG0634, GLPG0778, GLPG0974, Gluco, Glucocerin, glucosamine, glucosamine hydrochloride, glucosamine sulfate, Glucotin, Gludex, Glutilage, GLY079, GLY145, Glycanic, Glycefort up, Glygesic, Glysopep, GMCSF Antibody, GMI1010, GMI1011, GMI1043, GMR321, GN4001, Goanna Salve, Goflex, gold sodium thiomalate, golimumab, GP2013, GPCR modulator, GPR15 Antagonist, GPR183 antagonist, GPR32 antagonist, GPR83 antagonist, G-protein Coupled Receptor Antagonists, Graceptor, Graftac, granulocyte colony-stimulating factor antibody, granulocyte-macrophage colony-stimulating factor antibody, Gravx, GRC4039, Grelyse, GS101, GS9973, GSC100, GSK1605786, GSK1827771, GSK2136525, GSK2941266, GSK315234, GSK681323, GT146, GT442, Gucixiaotong, Gufisera, Gupisone, gusperimus hydrochloride, GW274150, GW3333, GW406381, GW856553, GWB78, GXPO4, Gynestrel, Haloart, halopredone acetate, Haloxin, HANALL, Hanall Soludacortin, Havisco, Hawon Bucillamin, HB802, HC31496, HCQ 200, HD104, HD203, HD205, HDAC inhibitor, HE2500, HE3177, HE3413, Hecoria, Hectomitacin, Hefasolon, Helen, Helenil, HemaMax, Hematom, hematopoietic stem cells, Hematrol, Hemner, Hemril, heparinoid, Heptax, HER2 Antibody, Herponil, hESC Derived Dendritic Cells, hESC Derived Hematopoietic stem cells, Hespercorbin, Hexacorton, Hexadrol, hexetidine, Hexoderm, Hexoderm Salic, HF0220, HF 1020, HFT-401, hG-CSFR ED Fc, Hiberna, high mobility group box 1 antibody, Hiloneed, Hinocam, hirudin, Hirudoid, Hison, Histamine H4 Receptor Antagonist, Hitenercept, Hizentra, HL036, HL161, HMPL001, HMPL004, HMPL011, HMPL342, HMPL692, honey bee venom, Hongqiang, Hotemin, HPH116, HTI101, HuCAL Antibody, Human adipose mesenchymal stem cells, anti-MHC class II monoclonal antibody, Human Immunoglobulin, Human Placenta Tissue Hydrolysate, HuMaxCD4, HuMax-TAC, Humetone, Humicade, Humira, Huons Betamethasone sodium phosphate, Huons dexamethasone sodium phosphate, Huons Piroxicam, Huons Talniflumate, Hurofen, Huruma, Huvap, HuZAF, HX02, Hyalogel, hyaluronate sodium, hyaluronic acid, hyaluronidase, Hyaron, Hycocin, Hycort, Hy-Cortisone, hydrocortisone, hydrocortisone acetate, hydrocortisone butyrate, hydrocortisone hemisuccinate, hydrocortisone sodium, phosphate, hydrocortisone sodium succinate, Hydrocortistab, Hydrocortone, Hydrolin, Hydroquine, Hydro-Rx, Hydrosone HIKMA, hydroxychloroquine, hydroxychloroquine sulfate, Hylase Dessau, HyMEX, Hypen, HyQ, Hysonate, HZN602, I.M.75, IAP Inhibitors, Ibalgin, Ibalgin, Ibex, ibrutinib, IBsolvMIR, Ibu, Ibucon, Ibudolor, Ibufen, Ibuflam, Ibuflex, Ibugesic, Ibu-Hepa, Ibukim, Ibumal, Ibunal, Ibupental, Ibupril, Ibuprof, ibuprofen, Ibuscent, Ibusoft, Ibusuki Penjeong, Ibususpen, Ibutard, Ibutop, Ibutrex, IC487892, ichthammol, ICRAC Blocker, IDEC131, IDECCE9.1, Ides, Idicin, Idizone, IDN6556, Idomethine, IDR1, Idyl SR, Ifen, iguratimod, IK6002, IKK-beta inhibitor, IL17 Antagonist, IL-17 Inhibitor, IL-17RC, IL18, IL1Hy1, IL1R1, IL-23 Adnectin, IL23 Inhibitor, IL23 Receptor Antagonist, IL-31 mAb, IL-6 Inhibitor, IL6Qb, Ilacox, Ilaris, ilodecakin, ILV094, 1LV095, Imaxetil, IMD0560, IMD2560, Irnesel Plus, Iminoral, Immodin, IMMUI03, IMMU106, Immucept, Immufine, Immunex Syrup, immunoglobulin, immunoglobulin G, Immunoprin, ImmunoRel, Immurin, IM08400, IMP731 antibody, Implanta, Imunocell, Imuran, Imurek, Imusafe, Imusporin, Imutrex, IN0701, Inal, INCB039110, INCB18424, INCB28050, INCB3284, INCB3344, Indexon, Indic, Indo, indo-A, Indobid, Indo-Bros, Indocaf, Indocarsil, Indocid, Indocin, Indomehotpas, Indomen, Indomet, Indometacin, indomethacin, Indomethasone, Indometin, Indomin, Indopal, Indoron, Indotroxin, INDUS830, INDUS83030, Infladase, Inflamac, Inflammasome inhibitor, Inflavis, Inflaxen, Inflectra, infliximab, Ingalipt, Inicox dp, Inmecin, Inmunoartro, Innamit, InnoD06006, IN07997, Inocin, Inoten, Inovan, Inpra, Inside Pap, Insider-P, Instacyl, Instracool, Intafenac, Intaflam, Inteban, Inteban Spansule, integrin, alpha 1 antibody, integrin, alpha 2 antibody, Intenurse, interferon alfa, interferon beta-la, interferon gamma, interferon gamma antibody, Interking, interleukin 1 Hyl, interleukin 1 antibody, interleukin 1 receptor antibody, interleukin 1 beta antibody, interleukin 10, interleukin 10 antibody, interleukin 12, interieukin 12 antibody, interleukin 13 antibody, interleukin 15 antibody, interleukin 17 antibody, interleukin 17 receptor C, interleukin 18, interleukin 18 binding protein, interleukin 18 antibody, interleukin 2 receptor, alpha antibody, interleukin 20 antibody, Interleukin 21 mAb, interleukin 23 aptamer, interleukin 31 antibody, interleukin 34, Interleukin 6 Inhibitor, interleukin 6 antibody, interleukin 6 receptor antibody, interleukin 7, interleukin 7 receptor antibody, interleukin 8, interleukin 8 antibody, interleukin-18 antibody, Intidrol, Intradex, Intragam P, Intragesic, Intraglobin F, Intratect, Inzel, Iomab B, IOR-T3, IP75I, IPH2201, IPH2301, IPH24, IPH33, IPI145, Ipocort, IPP201007, I-Profen, Iprox, Ipson, Iputon, IRAK4 Inhibitor, Iremod, Irtonpyson, IRX3, IRX5183, ISA247, ISIS104838, ISIS2302, ISISCRPRx, Ismafron, IsoQC inhibitor, Isox, ITF2357, Iveegam EN, Ivepred, WIG-SN, IW001, Izilox, J607Y, J775Y, JAK Inhibitor, JAK3 inhibitor, JAK3 kinase inhibitor, JI3292, JI4135, Jinan Lida, JNJ10329670, JNJ18003414, JNJ26528398, JNJ27390467, JNJ28838017, JNJ31001958, JNJ38518168, JNJ39758979, JNJ40346527, JNJ7777120, JNT-Plus, Joflam, Joint, Glucosamin, Jointec, Jointstem, Joinup, JPE1375, JSM10292, JSM7717, JSM8757, JTE051, JTE052, JTE522, JTE607, Jusgo, K412, K832, Kaflam, KAHR101, KAHR102, KAI9803, Kalymin, Kam Predsol, Kameton, KANAb071, Kappaproct, KAR2581, KAR3000, KAR3166, KAR4000, KAR4139, KAR4141, KB002, KB003, KD7332, KE298, keliximab, Kemanat, Kemrox, Kenacort, Kenalog, Kenaxir, Kenketsu Venoglobulin-IH, Keplat, Ketalgipan, Keto Pine, Keto, Ketobos, Ketofan, Ketofen, Ketolgan, Ketonal, Ketoplus Kata Plasma, ketoprofen, Ketores, Ketorin, ketorolac, ketorolac tromethamine, Ketoselect, Ketotop, Ketovail, Ketricin, Ketroc, Ketum, Keyi, Keyven, KF24345, K-Fenac, K-Fenak, K-Gesic, Kifadene, Kilcort, Kildrol, KIM127, Kimotab, Kinase Inhibitor 4SC, Kinase N, Kincort, Kindorase, Kineret, Kineto, Kitadol, Kitex, Kitolac, KLK1 inhibitor, Klofen-L, Klotaren, KLS-40or, KLS-40ra, KM277, Knavon, Kodolo orabase, Kohakusanin, Koide, Koidexa, Kolbet, Konac, Kondro, Kondromin, Konshien, Kontab, Kordexa, Kosa, Kotase, KPE06001, KRP107, KRP203, KRX211, KRX252, KSB302, K-Sep, Kv 1.3 Blocker, Kv 1.3 4SC, Kv1.3 inhibitor, KVK702, Kynol, L156602, Labizone, Labohydro, Labopen, Lacoxa, Lamin, Lamit, Lanfetil, laquinimod, larazotide acetate, LAS186323, LAS187247, LAS41002, Laticort, LBEC0101, LCP3301, LCP-Siro, LCP-Tacro, LCsA, LDP392, Leap-S, Ledercort, Lederfen, Lederlon, Lederspan, Lefenine, leflunomide, Leflux, Lefno, Lefra, Leftose, Lefumide, Lefunodin, Lefva, lenalidomide, lenercept, LentiRA, LEO15520, Leodase, Leukine, Leukocyte function-associated antigen-1 antagonist, leukocyte immunoglobulin-like receptor, subfamily A, member 4 antibody, Leukothera, leuprolide acetate, levalbuterol, levomenthol, LFA-1 Antagonist, LFA451, LFA703, LFA878, LG106, LG267 Inhibitors, LG688 Inhibitors, LGD5552, Li Life, LidaMantle, Lidex, lidocaine, lidocaine hydrochloride, Lignocaine hydrochloride, LIM0723, LIM5310, Limethason, Limus, Limustin, Lindac, Linfonex, Linola acute, Lipcy, lisofylline, Listran, Liver X Receptor modulator, Lizak, LJP1207, LJP920, Lobafen, Lobu, Locafluo, Localyn, Locaseptil-Neo, Locpren, Lodine, Lodotra, Lofedic, Loflani, Lofnac, Lolcam, Lonac, lonazolac calcium, Loprofen, Loracort, Lorcam, Lorfenamin, Lorinden Lotio, Lorncrat, lornoxicam, Lorox, losmapimod, loteprednol etabonate, Loteprednol, Lotirac, Low Molecular Ganoderma Lucidum Polysaccharide, Loxafen, Loxfenine, Loxicam, Loxofen, Loxonal, Loxonin, loxoprofen sodium, Loxoron, LP183A1, LP183A2, LP204A1, LPCN1019, LT1942, LT1964, LTNS101, LTNS103, LTNS106, LTNS108, LTS1115, LTZMP001, Lubor, lumiracoxib, Lumitect, LX2311, LX2931, LX2932, LY2127399, LY2189102, LY2439821, LY294002, LY3009104, LY309887, LY333013, lymphocyte activation gene 3 antibody, Lymphoglobuline, Lyser, lysine aspirin, Lysobact, Lysoflam, Lysozvme hydrochloride, M3000, M834, M923, mAb hG-CSF, MABP1, macrophage migration inhibitory factor antibody, Maitongna, Majamil prolongatum, major histocompatibility complex class II DR antibody, major histocompatibility complex class II antibody, Malidens, Malival, mannan-binding lectin, mannan-binding lectin-associated serine protease-2 antibody, MapKap Kinase 2 Inhibitor, maraviroc, Marlex, masitinib, Maso, MASP2 antibody, MAT304, Matrix Metalloprotease Inhibitor, mavrilimumab, Maxiflam, Maxilase, Maximus, Maxisona, Maxius, Maxpro, Maxrel, Maxsulid, Maxyl 2, Maxy30, MAXY4, Maxy735, Maxy740, Mayfenamic, MB11040, MBPY003b, MCAF5352A, McCam, McRofy, MCS18, MD707, MDAM, MDcort, MDR06155, MDT012, Mebicam, Mebuton, meclofenamate sodium, Meclophen, Mecox, Medacomb, Medafen, Medamol, Medesone, MEDI2070, MEDI5117, MEDI541, MED1552, MEDI571, Medicox, Modifen, Medisolu, Medixon, Mednisol, Medrol, Medrolon, medroxyprogesterone acetate, Mefalgin, mefenamic acid, Mefenix, Mefentan, Meflen, Mefnetra forte, Meftagesic-DT, Meftal, Megakaryocyte Growth and Development Factor, Megaspas, Megaster, megestrol acetate, Meite, Meksun, Melbrex, Melcam, Melflam, Melic, Melica, Melix, Melocam, Melocox, Mel-One, Meloprol, Melosteral, Melox, Meloxan, Meloxcam, Meloxic, Meloxicam, Meloxifen, Meloxin, Meloxiv, Melpred, Melpros, Melurjin, Menamin, Menisone, Menthomketo, Menthoneurin, Mentocin, Mepa, Mepharen, meprednisone, Mepresso, Mepsolone, mercaptopurine, Mervan, Mesadoron, mesalamine, Mesasal, Mesatec, Mesenchymal Precursor Cells, mesenchymal stem cell, Mesipol, Mesren, Mesulan, Mesulid, Metacin, Metadaxan, Metaflex, Metalcaptase, metalloenzyme inhibitors, Metapred, Metax, Metaz, Meted, Metedic, Methacin, Methaderm, Methasone, Methotrax, methotrexate, methotrexate sodium, Methpred, Methyl prednisolone acetate, methyl salicylate, methyl sulphonyl methane, Methylon, Methylpred, methylprednisolone, methylprednisolone acetate, methylprednisolone sodium succinate, methylprednisolone succinate, Methysoi, Metindol, Metoart, Metoject, Metolate, Metoral, Metosyn, Metotab, Metracin, Metrex, metronidazole, Metypred, Mevamox, Mevedal, Mevilox, Mevin SR, Mexilal, Mexpharm, Mext, Mextran, MF280, M-FasL, MHC class II beta chain peptide, Micar, Miclofen, Miclofenac, Micofenolato Mofetil, Micosone, Microdase, microRNA 181a-2 oligonucleotide, MIF Inhibitors, MIFQb, MIKA-Ketoprofen, Mikametan, milodistim, Miltax, Minafen, Minalfen, Minalfene, Minesulin, Minocort, Mioflex, Miolox, Miprofen, Miridacin, Mirloks, Misoclo, Misofenac, MISTB03, M1STB04, Mitilor, mizoribine, MK0359, MK0812, MK0873, MK2 Inhibitors, MK50, MK8457, MK8808, MKC204, MLN0002, MLN0415, MLN1202, MLN273, MLN3126, MLN3701, MLN3897, MLNM002, MM093, MM7XX, MN8001, Mobic, Mobicam, Mobicox, Mobifen Plus, Mobilat, Mobitil, Mocox, Modigraf, Modrasone, Modulin, Mofecept, Mofetyl, mofezolac sodium, Mofilet, Molace, molgramostim, Molslide, Momekin, Momen Gele, Moment 500, Momesone, Momesun, Mometamed, mometasone, mometasone furoate, Monimate, monosodium alpha-luminol, Mopik, MOR103, MOR104, MOR105, MOR208 antibody, MORAb022, Moricam, momiflumate, Mosuolit, Motoral, Movaxin, Mover, Movex, Movix, Movoxicarn, Mox Forte, Moxen, moxifloxacin hydrochloride, Mozobil, MP, MP0210, MP0270, MP1000, MP 1031, MP196, MP435, MPA, mPGES-1 inhibitor, MPSS, MRX7EAT, MSL, MT203, MT204, mTOR Inhibitor, MTRX1011A, Mucolase, Multicort, MultiStem, muramidase, muramidase hydrochloride, muromonab-CD3, Muslax, Muspinil, Mutaze, Muvera, MX68, Mycept, Mycocell, Mycocept, Mycofenolatmofetil Actavis, Mycofet, Mycofit, Mycolate, Mycoldosa, Mycomun, Myconol, mycophenolate mofetil, mycophenolate sodium, mycophenolic acid, Mycotil, myeloid progenitor cells, Myfenax, Myfetil, Myfortic, Mygraft, Myochrysine, Myocrisin, Myprodol, Mysone, nab-Cyclosporine, Nabentac, nabiximols, Nabton, Nabuco, Nabucox, Nabuflam, Nabumet, nabumetone, Nabuton, Nac Plus, Nacta, Nacton, Nadium, Naklofen SR, NAL1207, NAL1216, NAL1219, NAL1268, NAL8202, Nalfon, Nalgesin S, namilumab, Namsafe, nandrolone, Nanocort, Nanogam, Nanosomal Tacrolimus, Napageln, Napilac, Naprelan, Napro, Naprodil, Napronax, Napropal, Naproson, Naprosyn, Naproval, Naprox, naproxen, naproxen sodium, Naproxin, Naprozen, Narbon, Narexsin, Naril, Nasida, natalizumab, Naxdom, Naxen, Naxin, Nazovel, NC2300, ND07, NDC01352, Nebumetone, NecLipGCSF, Necsulide, Necsunim, Nelsid-S, Neo Clobenate, Neo Swiflox FC, Neocoflan, Neo-Drol, Neo-Eblimon, Neo-Hydro, Neoplanta, Neoporine, Neopreol, Neoprox, Neoral, Neotrexate, Neozen, Nepra, Nestacort, Neumega, Neupogen, Neuprex, Neurofenac, Neurogesic, Neurolab, Neuroteradol, Neuroxicam, Neutalin, neutrazumab, Neuzym, New Panazox, Newfenstop, NewGam, Newmafen, Newmatal, Newsicam, NEX1285, sFcRIIB, Nextomab, NF-kappaB Inhibitor, NGD20001, NHP554B, NHP554P, NI0101 antibody, NI0401, NI0501 antibody, NI0701, NI071, NI1201 antibody, NI1401, Nicip, Niconas, Nicool, NiCord, Nicox, Niflumate, Nigaz, Nikam, Nilitis, Nimace, Nimaid, Nimark-P, Nimaz, Nimcet Juicy, Nime, Nimed, Nimepast, nimesulide, Nimesulix, Nimesulon, Nimica Plus, Nimkul, Nimlin, Nimnat, Nimodol, Nimpidase, Nimsaid-S, Nimser, Nimsy-SP, Nimupep, Nimusol, Nimutal, Nimuwin, Nimvon-S, Nincort, Niofen, Nipan, Nipent, Nise, Nisolone, Nisopred, Nisoprex, Nisulid, nitazoxanide, Nitcon, nitric oxide, Nizhvisal B, Nizon, NL, NMR1947, NN8209, NN8210, NN8226, NN8555, NN8765, NN8828, NNV014100000100, NNCO51869, Noak, Nodevex, Nodia, Nofenac, Noflagma, Noflam, Noflamen, Noflux, Non-antibacterial Tetracyclines, Nonpiron, Nopain, Normferon, Notpel, Notritis, Novacort, Novagent, Novarin, Novigesic, NOXA12, NOXD19, Noxen, Noxon, NPI1302a-3, NP1342, NPI1387, NPI1390, NPRCS1, NPRCS2, NPRCS3, NPRCS4, NPRCSS, NPRCS6, NPS3, NPS4, nPT-ery, NU3450, nuclear factor NF-kappa-B p65 subunit oligonucleotide, Nucort, Nulojix, Numed-Plus, Nurokind Ortho, Nusone-H, Nutrikemia, Nuvion, NVO7alpha, NX001, Nyclobate, Nyox, Nysa, Obarcort, OC002417, OC2286, ocaratuzumab, OCTSG815, Oedemase, Oedemase-D, ofatumumab, Ofgy1-O, Ofvista, OHR118, OKi, Okifen, Oksamen, Olai, olokizumab, Omeprose E, Omnacortil, Omneed, Omniclor, Omnigel, Omniwel, onercept, ONO4057, ONS1210, ONS1220, Ontac Plus, Ontak, ONX0914, OPC6535, opebacan, OPN101, OPN201, OPN302, OPN305, OPN401, oprelvekin, OPT66, Optifer, Optiflur, OptiMIRA, Orabase Hca, Oradexon, Oraflex, OralFenac, Oralog, Oralpred, Ora-sed, Orasone, orBec, Orbone forte, Orel, ORE10002, Orencia, Org214007, Org217993, Org219517, Org223119, Org37663, Org39141, Org48762, Org48775, Orgadrone, Ormoxen, Orofen Plus, Oromylase Biogaran, Orthal Forte, Ortho Flex, Orthoclone OKT3, Orthofen, Orthoflam, Orthogesic, Orthoglu, Ortho-II Orthomac, Ortho-Plus, Ortinims, Ortofen, Orudis, Oruvail, OS2, Oscart, Osmetone, Ospain, Ossilife, Ostelox, Osteluc, Osteocerin, osteopontin, Osteral, otelixizumab, Otipax, Ou Ning, OvaSave, OX40 Ligand Antibody, Oxa, Oxagesic CB, Oxalgin DP, oxaprozin, OXCQ, Oxeno, Oxib MD, Oxibut, Oxicam, Oxiklorin, Oximal, Oxynal, oxyphenbutazone, ozoralizumab, P13 peptide, P1639, P21, P2X7 Antagonists, p38 Alpha Inhibitor, p38 Antagonist, p38 MAP kinase inhibitor, p38alpha MAP Kinase Inhibitor, P7 peptide, P7170, P979, PA40I, PA517, Pabi-dexamethasone, PAC, PAC10649, paclitaxel, Painoxam, Paldon, Palima, pamapimod, Pamatase, Panafcort, Panafcortelone, Panewin, PanGraf, Panimun Bioral, Panmesone, Panodin SR, Panslay, Panzem, Panzem NCD, PAP1, papain, Papirzin, Pappen K Pap, Paptinim-D, paquinimod, PAR2 Antagonist, Paracetamol, Paradic, Parafen TAJ, Paramidin, Paranac, Parapar, Parci, parecoxib, Parixam, Parry-S, Partaject Busulfan, pateclizumab, Paxceed, PBI0032, PBI1101, PBI1308, PBI1393, PBI1607, PBI1737, PBI2856, PBI4419, P-Cam, PCI31523, PCI32765, PCI34051, PCI45261, PCI45292, PCI45308, PD360324, PDA001, PDE4 inhibitor, PDL241 antibody, PDL252, Pediapred, Pefree, pegacaristim, Peganix, Peg-Interleukin 12, pegsunercept, PEGylated arginine deiminase, peldesine, pelubiprofen, Penacle, penicillamine, Penostop, Pentalgin, Pentasa, Pentaud, pentostatin, Peon, Pepdase, Pepser, Peptirase, Pepzen, Pepzol, Percutalgine, Periochip, Peroxisome Proliferator Activated Receptor gamma modulators, Petizene, PF00344600, PF04171327, PF04236921, PF04308515, PF05230905, PF05280586, PF251802, PF3475952, PF3491390, PF3644022, PF4629991, PF4856880, PF5212367, PF5230896, PF547659, PF755616, PF9184, PG27, PG562, PG760564, PG8395, PGE3935199, PGE527667, PHS, PH797804, PHA408, Pharmaniaga Mefenamic acid, Pharmaniaga Meloxicam, Pheldin, Phenocept, phenylbutazone, PHY702, PI3K delta inhibitor, PI3 Gamma/Delta Inhibitor, PI3K Inhibitor, Picalm, pidotimod, piketoprofen, Pilelife, Pilopil, Pilovate, pimecrolimus, Pipethanen, Piractam, Pirexyl, Pirobet, Piroc, Pirocam, Pirofel, Pirogel, Piromed, Pirosol, Pirox, Piroxen, Piroxicam, piroxicam betadex, Piroxifar, Piroxil, Piroxim, Pixim, Pixykine, PKC Theta Inhibitor, PL3100, PL5100 Diclofenac, Placenta Polypeptide, Plaquenil, plerixafor, Plocfen, PLR14, PLR18, Plutin, PLX3397, PLX5622, PLX647, PLX-BMT, pms-Diclofenac, pms-Ibuprofen, pms-Leflunomide, pms-Meloxicam, pms-Piroxicam, pms-Prednisolone, pms-Sulfasalazine, pms-Tiaprofenic, PMX53, PN0615, PN100, PN951, podofilox, POL6326, Polcortolon, Polyderm, Polygam S/D, Polyphlogin, Poncif, Ponstan, Ponstil Forte, Porine-A Neoral, Potaba, potassium aminobenzoate, Potencort, Povidone, povidone iodine, pralnacasan, Prandin, Prebel, Precodil, Precortisyl Forte, Precortyl, Predfoam, Predicort, Predicorten, Predilab, Predilone, Predmetil, Predmix, Predna, Prednesol, Predni, prednicarbate, Prednicort, Prednidib, Prednifarma, Prednilasea, prednisolone, Deltacortril (prednisolone), prednisolone acetate, prednisolone sodium phosphate, prednisolone sodium succinate, prednisone, prednisone acetate, Prednitop, Prednol-L, Prednox, Predone, Predonema, Predsol, Predsolone, Predsone, Predval, Preflam, Prelon, Prenaxol, Prenolone, Preservex, Preservin, Presol, Preson, Prexige, Priliximab, Primacort, Primmuno, Primofenac, prinaberel, Privigen, Prixam, Probuxil, Procarne, Prochymal, Procider-EF, Proctocir, Prodase, Prodel B, Prodent, Prodent Verde, Proepa, Profecom, Profenac L, Profenid, Profenol, Proflam, Proflex, Progesic Z, proglumetacin, proglumetacin maleate, Prograf, Prolase, Prolixan, promethazine hydrochloride, Promostem, Promune, PronaB, pronase, Pronat, Prongs, Pronison, Prontoflam, Propaderm-L, Propodezas, Propolisol, Proponol, propyl nicotinate, Prostaloc, Prostapol, Protacin, Protase, Protease Inhibitors, Protectan, Proteinase Activated Receptor 2 Inhibitor, Protofen, Protrin, Proxalyoc, Proxidol, Proxigel, Proxil, Proxym, Prozym, PRT062070, PRT2607, PRTX100, PRTX200, PRX106, PRX167700, Prysolone, PS031291, PS375179, PS386113, PS540446, PS608504, PS826957, PS873266, Psorid, PT, PT17, PTL101, P-Transfer Factor peptides, PTX3, Pulminiq, Pulsonid, Purazen, Pursin, PVS40200, PX101, PX106491, PX114, PXS2000, PXS2076, PYM60001, Pyralvex, Pyranim, pyrazinobutazone, Pyrenol, Pyricam, Pyrodex, Pyroxi-Kid, QAX576, Qianbobiyan, QPI1002, QR440, qT3, Quiacort, Quidofil, R107s, R125224, R1295, R132811, R1487, R1503, R1524, R1628, R333, R348, R548, R7277, R788, rabeximod, Radix Isatidis, Radofen, Raipeck, Rambazole, Randazima, Rapacan, Rapamune, Raptiva, Ravax, Rayos, RDEA119, RDEA436, RDP58, Reactine, Rebif, REC200, Recartix-DN, receptor for advanced glycation end products antibody, Reclast, Reclofen, recombinant HSA-TTMP-2, recombinant human alkaline phosphatase, recombinant Interferon Gamma, Recombinant human alkaline phosphatase, Reconil, Rectagel HC, Recticin, Recto Menaderm, Rectos, Redipred, Redolet, Refastin, Regenica, REGN88, Relafen, Relaxib, Relev, Relex, Relifen, Relifex, Relitch, Rematof, remestemce1-1, Remesulidum, Remicade® (infliximab), Remsima, ReN1869, Renacept, Renfor, Renodapt, Renodapt-S, Renta, Reosan, Repare-AR, Reparilexin, reparixin, Repertaxin, Repisprin, Resochin, Resol, resolvin E1, Resurgil, Re-tin-colloid, Retoz, Reumacap, Reumacon, Reumadolor, Reumador, Reumanisal, Reumazin, Reumel, Reumotec, Reuquinol, revamilast, Revascor, Reviroc, Revlimid, Revmoksikam, Rewalk, Rexalgan, RG2077, RG3421, RG4934 antibody, RG7416, RG7624, Rheila, Rheoma, Rheprox, Rheudenolone, Rheufen, Rheugesic, Rheumacid, Rheumacort, Rheumatrex, Rheumesser, Rheumid, Rheumon, Rheumox, Rheuoxib, Rhewlin, Rhucin, RhuDex, Rhulef, Ribox, Ribunal, Ridaura, rifaximin, rilonacept, rimacalib, Rimase, Rimate, Rimatil, Rimesid, risedronate sodium, Ritamine, Rito, Rituxan, rituximab, RNS60, RO1138452, Ro313948, RO3244794, RO5310074, Rob803, Rocamix, Rocas, Rofeb, rofecoxib, Rofee, Rofewal, Roficip Plus, Rojepen, Rokam, Rolodiquim, Romacox Fort, Romatim, romazarit, Ronaben, ronacaleret, Ronoxcin, RDR Gamma T Antagonist, ROR gamma t inverse agonists, Rosecin, rosiglitazone, Rosmarinic acid, Rotan, Rotec, Rothacin, Roxam, Roxib, Roxicam, Roxopro, Roxygin DT, RP54745, RPI78, RPI78M, RPI78MN, RPIMN, RQ00000007, RQ00000008, RTA402, R-Tyflam, Rubicalm, Rubifen, Ruma pap, Rumalef, Rumidol, Rumifen, Runomex, rusalatide acetate, ruxolitinib, RWJ445380, RX10001, Rycloser MR, Rydol, S1P Receptor Agonists, S1P Receptor Modulators, S1P1 Agonist, S1P1 receptor agonist, S2474, S3013, SA237, SA6541, Saaz, S-adenosyl-L-methionine-sulfate-p-toluene sulfonate, Sala, Salazidin, Salazine, Salazopyrin, Salcon, Salicam, salsalate, Sameron, SAN300, Sanaven, Sandimmun, Sandoglobulin, Sanexon, SangCya, SAR153191, SAR302503, SAR479746, Sarapep, sargramostim, Sativex, Savantac, Save, Saxizon, Sazo, SB1578, SB210396, SB217969, SB242235, SB273005, SB281832, SB683698, SB751689, SBI087, SC080036, SC12267, SC409, Scaflam, SCD ketoprofen, SCIO323, SCIO469, SD-15, SD281, SDP051 antibody, Sd-rxRNA, secukinumab, Sedase, Sedilax, Sefdene, Seizyme, SEL113, Seladin, Selecox, selectin P ligand antibody, Glucocorticoid Receptor Agonist, Selectofen, Selektine, SelK1 antibody, Seloxx, Selspot, Selzen, Selzenta, Selzentry, semapimod, semapimod hydrochloride, semparatide, Senafen, Sendipen, Senterlic, SEP119249, Sepdase, Septirose, Seractil, Serafen-P, Serase, Seratid D, Seratiopeptidase, Serato-M, Seratoma Forte, Serazyme, Serezon, Sero, Serodase, Serpicam, Serra, serrapeptase, Serratin, Serratiopeptidase, Serrazyme, Servisone, Seven E P, SGI1252, SGN30, SGN70, SGX203, shark cartilage extract, Sheril, Shield, Shifazen, Shifazen-Fort, Shincort, Shiosol, ShK186, Shuanghuangxiaoyan, SI615, SI636, Sigmasporin, SIM916, Simpone, Simulect, Sinacort, Sinalgia, Sinapol, Sinatrol, Sinsia, siponimod, Sirolim, sirolimus, Siropan, Sirota, Sirova, sirukmnab, Sistal Forte, SKF105685, SKF105809, SKF106615, SKF86002, Skinalar, Skynim, Skytrip, SLAM family member 7 antibody, Slo-indo, SM101, SM201 antibody, SM401, SMAD family member 7 oligonucleotide, SMART Anti-IL-12 Antibody, SMP114, SNO030908, SNO070131, sodium aurothiomalate, sodium chondroitin sulfate, sodium deoxyribonucleotide, sodium gualenate, sodium naproxen, sodium salicylate, Sodixen, Sofeo, Soleton, Solhidrol, Solicam, Soliky, Soliris, Sol-Melcort, Solomet, Solondo, Solone, Solu-Cort, Solu-Cortef, Solu-Decortin H, Solufen, Solu-Ket, Solumark, Solu-Medrol, Solupred, Somalgen, somatropin, Sonap, Sone, sonepeizumab, Sonexa, Sonim, Sonim P, Soonil, Soral, Sorenil, sotrastaurin acetate, SP-10, SP600125, Spanidin, SP-Cortil, SPD550, Spedace, sperm adhesion molecule 1, Spictol, spleen tyrosine kinase oligonucleotide, Sporin, S-prin, SPWF1501, SQ641, SQ922, SR318B, SR9025, SRT2104, SSR150106, SSR180575, SSS07 antibody, ST1959, STA5326, stabilin 1 antibody, Stacort, Stalogesic, stanozolol, Staren, Starmelox, Stedex IND-SWIFT, Stelara, Stemin, Stenirol, Sterapred, Steriderm S, Sterio, Sterisone, Steron, stichodactyla helianthus peptide, Stickzenol A, Stiefcortil, Stimulan, STNM01, Store Operated Calcium Channel (SOCC) Modulator, STP432, STP900, Stratasin, Stridimmune, Strigraf, SU Medrol, Subreum, Subuton, Succicort, Succimed, Sulan, Sulcolon, Sulfasalazin Heyl, Sulfasalazin, Sulfovit, Sulidac, Sulide, sulindac, Sulindex, Sulinton, Sulphafine, Surnilu, SUN597, Suprafen, Supretic, Supsidine, Surgam, Surgamine, Surugamu, Suspen, Suton, Suvenyl, Suwei, SW Dexasone, Syk Family Kinase Inhibitor, Syn1002, Synacran, Synacthen, Synalar C, Synalar, Synavive, Synercort, Sypresta, T cell cytokine-inducing surface molecule antibody, T cell receptor antibody, T5224, T5226, TA101, TA112, TA383, TA5493, tabalumab, Tacedin, Tacgraf, TACIFc5, Tacrobell, Tacrograf, Tacrol, tacrolimus, Tadekinig alpha, Tadolak, TAFA93, Tafirol Artro, Taizen, TAK603, TAK715, TAK783, Takfa, Taksta, talarozole, Talfin, Talmain, talmapimod, Talmea, Talnif, talniflumate, Talos, Talpain, Talumat, Tamalgen, Tamceton, Tamezon, Tandrilax, tannins, Tannosynt, Tantum, tanzisertib, Tapain-beta, Tapoein, Tarenac, tarenflurbil, Tarimus, Tarproxen, Tauxib, Tazomust, TBR652, TC5619, T-cell, immune regulator 1, ATPase, H+ transporting, lysosomal V0 subunit A3 antibody, TCK1, T-cort, T-Dexa, Tecelac, Tecon, teduglutide, Teecort, Tegeline, Tementil, temoporfin, Tencam, Tendrone, Tenefuse, Tenfly, tenidap sodium, Tenocam, Tenoflex, Tenoksan, Tenotil, tenoxicam, Tenoxim, Tepadina, Teracort, Teradol, tetomilast, TG0054, TG1060, TG20, TG20, tgAAC94, Th1/Th2 Cytokine Synthase Inhibitor, Th-17 cell inhibitors, Thalido, thalidomide, Thalomid, Themisera, Thenii, Therafectin, Therapyace, thiarabine, Thiazolopyrimi dines, thioctic acid, thiotepa, THR090717, THR0921, Threenofen, Thrombate III, Thymic peptide, Thymodepressin, Thymogam, Thymoglobulin, Thymoglobuline, Thymoject thymic peptides, thymoniodulin, thymopentin, thymopolypetides, tiaprofenic acid, tibezonium iodide, Ticoflex, tilmacoxib, Tilur, T-immune, Timocon, Tiorase, Tissop, TKB662, TL011, TLR4 antagonists, TLR8 inhibitor, TM120, TM400, TMX302, TNF Alpha inhibitor, TNF alpha-TNF receptor antagonist, TNF antibody, TNF receptor superfamily antagonists, TNF TWEAK Bi-Specific, TNF-Kinoid, TNFQb, TNFR1 antagonist, TNR001, TNX100, TNX224, TNX336, TNX558, tocilizumab, tofacitinib, Tokuhon happ, TOL101, TOL102, Tolectin, ToleriMab, Tolerostem, Tolindol, toll-like receptor 4 antibody, toll-like receptor antibody, tolmetin sodium, Tongkeeper, Tonmex, Topflame, Topicort, Topleucon, Topnac, Toppin Ichthammol, toralizumab, Toraren, Torcoxia, Toroxx, Tory, Toselac, Totaryl, Touch-med, Touchron, Tovok, Toxic apis, Toyolyzom, TP4179, TPCA1, TPI526, TR14035, Tradil Fort, Traficet-EN, Tramace, tramadol hydrochloride, tranilast, Transimune, Transporina, Tratul, Trexall, Triacort, Triakort, Trialon, Triam, triamcinolone, triamcinolone acetate, triamcinolone acetonide, triamcinolone acetonide acetate, triamcinolone hexacetonide, Triamcort, Triamsicort, Trianex, Tricin, Tricort, Tricortone, TricOs T, Triderm, Trilac, Trilisate, Trinocort, Trinolone, Triolex, triptolide, Trisfen, Trivaris, TRK170, TRK530, Trocade, trolamine salicylate, Trolovol, Trosera, Trosera D, Trovcort, TRX1 antibody, TRX4, Trymoto, Trymoto-A, TT301, TT302, TT32, TT33, TTI314, tumor necrosis factor, tumor necrosis factor 2-methoxyethyl phosphorothioate oligonucleotide, tumor necrosis factor antibody, tumor necrosis factor kinoid, tumor necrosis factor oligonucleotide, tumor necrosis factor receptor superfamily, member I B antibody, tumor necrosis factor receptor superfamily1B oligonucleotide, tumor necrosis factor superfamily, member 12 antibody, tumor necrosis factor superfamily, member 4 antibody, tumor protein p53 oligonucleotide, tumour necrosis factor alpha antibody, TuNEX, TXA127, TX-RAD, TYK2 inhibitors, Tysabri, ubidecarenone, Ucerase, ulodesine, Ultiflam, Ultrafastin, Ultrafen, Ultralan, U-Nice-B, Uniplus, Unitrexate, Unizen, Uphaxicam, UR13870, UR5269, UR67767, Uremol-HC, Urigon, U-Ritis, ustekinumab, V85546, Valcib, Valcox, valdecoxib, Yaldez, Valdixx, Valdy, Valentac, Vaioxib, Valtune, Valus AT, Valz, Valzer, Vamid, Vantal, Vantelin, VAP-1 SSAO Inhibitor, vapaliximab, varespladib methyl, Varicosin, Varidase, vascular adhesion protein-1 antibody, VB110, VB120, VB201, VBY285, Vectra-P, vedolizumab, Vefren, VEGFR-1 Antibody, Veldona, veltuzumab, Vendexine, Venimmun N, Veno forte, Venoglobulin-IH, Venozel, Veral, Verax, vercirnon, vero-dexamethasone, Vero-Kladribin, Vetazone, VGX1027, VGX750, Vibex MTX, vidofludimus, Vifenac, Vimovo, Vimultisa, Vincort, Vingraf, Vioform-HC, Vioxl, Vioxx, Virobron, visilizumab, Vivaglobin, Vivalde Plus, Vivian-A, VLST002, VLST003, VLST004, VLST005, VLST007, Voalla, voclosporin, Vokam, Vokmor, Volmax, Volna-K, Voltadol, Voltagesic, Voltanase, Voltanec, Voltaren, Voltarile, Voltic, Voren, vorsetuzumab, Votan-SR, VR909, VRA002, VRP1008, VRS826, VT111, VT214, VT224, VT310, VT346, VT362, VTX763, Vurdon, VX30 antibody, VX467, VXS, VX509, VX702, VX740, VX745, VX850, W54011, Walacort, Walix, WC3027, Wilgraf, Winflam, Winmol, Winpred, Winsolve, Wintogeno, WIP901, Woncox, WSB711 antibody, WSB712 antibody, WSB735, WSB961, X071NAB, X083NAB, Xantomicin Forte, Xedenol, Xefo, Xefocam, Xenar, Xepol, X-Flam, Xibra, Xicam, Xicotil, Xifaxan, XL499, XmAb5483, XmAb5485, XmAb5574, XmAb5871, XOMA052, Xpress, XProl 595, XtendTNF, XToll, Xtra, Xylex-H, Xynofen SR, Yang Shu-IVIG, YHB14112, YM974, Youfeline, Youfenac, Yuma, Yumerol, Yuroben, YY piroxicam, Z104657A, Zacy, Zaltokin, zaltoprofen, Zap70 Inhibitor, Zeepain, Zeloxim Fort, Zema-Pak, Zempack, Zempred, Zenapax, Zenas, Zenol, Zenos, Zenoxone, Zerax, Zerocam, Zerospasm, ZFNs, zinc oxide, Zipsor, ziralimumab, Zitis, Zix-S, Zocort, Zodixam, Zoftadex, zoledronic acid, Zolfin, Zolterol, Zopyrin, Zoralone, ZORprin, Zortress, ZP1848, zucapsaicin, Zunovate, Zwitterionic polysaccharides, ZY1400, Zybodies, Zycel, Zyrofen, Zyrogen Inhibitors, Zyser, Zytrim, and Zywin-Forte. In addition, the anti-inflammatory drugs, as listed above, may be combined with one or more agents listed above or herein or with other agents known in the art.

In one embodiment, the anti-inflammatory drug is non-surgically delivered to the SCS of the eye using the microneedle devices and methods disclosed herein, and is used to treat, prevent and/or ameliorate a posterior ocular disorder in a human patient in need thereof. For example, the posterior ocular disorder or disorder selected from macular degeneration (e.g., age related macular degeneration, dry age related macular degeneration, exudative age-related macular degeneration, geographic atrophy associated with age related macular degeneration, neovascular (wet) age-related macular degeneration, neovascular maculopathy and age related macular degeneration, occult with no classic choroidal neovascularization (CNV) in age-related macular degeneration, Stargardt's disease, subfoveal wet age-related macular degeneration, and Vitreomacular Adhesion (VMA) associated with neovascular age related macular degeneration), macular edema, diabetic macular edema, uveitis, scleritis, chorioretinal inflammation, chorioretinitis, choroiditis, retinitis, retinochoroiditis, focal chorioretinal inflammation, focal chorioretinitis, focal choroiditis, focal retinitis, focal retinochoroiditis, disseminated chorioretinal inflammation, disseminated chorioretinitis, disseminated choroiditis, disseminated retinitis, disseminated reinochoroiditis, posterior cyclitis, Harada's disease, chorioretinal scars (e.g., macula scars of posterior pole, solar retinopathy), choroidal degeneration (e.g., atrophy, sclerosis), hereditary choroidal dystrophy (e.g., choroidermia, choroidal dystrophy, gyrate atrophy), choroidal hemorrhage and rupture, choroidal detachment, retinal detachment, retinoschisis, hypersentitive retinopathy, retinopathy, retinopathy of prematurity, epiretinal membrane, peripheral retinal degeneration, hereditary retinal dystrophy, retinitis pigmentosa, retinal hemorrhage, separation of retinal layers, central serous retinopathy, glaucoma, ocular hypertension, glaucoma suspect, primary open-angle glaucoma, primary angle-closure glaucoma, floaters, Leber's hereditary optic neropathy, optic disc drusen, inflammatory disorders of the eye, inflammatory lesions in fungal infections, inflammatory lesions, inflammatory pain, inflammatory skin diseases or disorders, Sjogren's syndrome, opthalmic for Sjogren's syndrome.

Examples of drugs that may be used to treat, prevent, and/or ameliorate macular degeneration that can be delivered to the SCS via the formulations and methods described herein include, but are not limited to: A0003, A36 peptide, AAV2-sFLT01, ACE041, ACU02, ACU3223, ACU4429, AdPEDF, aflibercept, AG13958, aganirsen, AGN150998, AGN745, AL39324, AL78898A, AL8309B, ALN-VEG01, alprostadil, AM1101, amyloid beta antibody, anecortave acetate, Anti-VEGFR-2 Alterase, Aptocine, APX003, ARC 1905, ARC 1905 with Lucentis, ATG3, ATP-binding cassette, sub-family A, member 4 gene, ATXS10, Avastin with Visudyne, AVT1O1, AVT2, bertilimumab, bevacizumab with verteporfin, bevasiranib sodium, bevasiranib sodium with ranibizumab, brimonidine tartrate, BVA301, canakinumab, Cand5, Cand5 with Lucentis, CERE 140, ciliary neurotrophic factor, CLT009, CNT02476, collagen monoclonal antibody, complement component 5 aptamer (pegylated), complement component 5 aptamer (pegylated) with ranibizumab, complement component C3, complement factor B antibody, complement factor D antibody, copper oxide with lutein, vitamin C, vitamin E, and zinc oxide, dalantercept, DE109, bevacizumab, ranibizumab, triamcinolone, triamcinolone acetonide, triamcinolone acetonide with verteporfin, dexamethasone, dexamethasone with ranibizumab and verteporfin, disitertide, DNA damage inducible transcript 4 oligonucleotide, E10030, E10030 with Lucentis, EC400, eculizumab, EGP, EHT204, embryonic stem cells, human stem cells, endoglin monoclonal antibody, EphB4 RTK Inhibitor, EphB4 Soluble Receptor, ESBA1008, ETX6991, Evizon, Eyebar, EyePromise Five, Eyevi, Eylea, F200, FCFD4514S, fenretinide, fluocinolone acetonide, fluocinolone acetonide with ranibizumab, fms-related tyrosine kinase 1 oligonucleotide, fms-related tyrosine kinase 1 oligonucleotide with kinase insert domain receptor 169, fosbretabulin tromethamine, Gamunex, GEM220, GS101, GSK933776, HC31496, Human n-CoDeR, HYB676, IBI-20089 with ranibizumab (Lucentis®), iCo-008, Icon1, I-Gold, Ilaris, Iluvien, Iluvien with Lucentis, immunoglobulins, integrin alpha5beta1 immunoglobulin fragments, Integrin inhibitor, IRIS Lutein, I-Sense Ocushield, Isonep, isopropyl unoprostone, JPE1375, JSM6427, KH902, LentiVue, LFG316, LP590, LPO1010AM, Lucentis, Lucentis with Visudyne, Lutein ekstra, Lutein with myrtillus extract, Lutein with zeaxanthin, M200, M200 with Lucentis, Macugen, MC1101, MCT355, mecamylamine, Microplasmin, motexafin lutetium, MP0112, NADPH oxidase inhibitors, aeterna shark cartilage extract (Arthrovas™, Neoretna™, Psovascar™), neurotrophin 4 gene, Nova21012, Nova21013, NT501, NT503, Nutri-Stulln, ocriplasmin, OcuXan, Oftan Macula, Optrin, ORA102 with bevaciziunab (Avastin®), P144, P17, Palomid 529, PAN90806. Panzem, PARP inhibitors, pazopanib hydrochloride, pegaptanib sodium, PF4523655, PG11047, piribedil, platelet-derived growth factor beta polypeptide aptamer (pegylated), platelet-derived growth factor beta polypeptide aptamer (pegylated) with ranibizumab, PLG101, PMX20005, PMX53, POT4, PRS055, PTK787, ranibizumab, ranibizumab with triamcinolone acetonide, ranibizumab with verteporfin, ranibizumab with volociximab, RD27, Rescula, Retaane, retinal pigment epithelial cells, RetinoStat, RG7417, RN6G, RT101, RTU007, SB267268, serpin peptidase inhibitor, clade F, member 1 gene, shark cartilage extract, Shef1, SIR1046, SIR1G76, Sirna027, sirolimus, SMTD004, Snelvit, SOD Mimetics, Solaris, sonepcizumab, squalamine lactate, ST602, StarGen, T2TrpRS, TA106, talaporfin sodium, Tauroursodeoxycholic acid, TG100801, TK1, TLCx99, TRC093, TRC105, Trivastal Retard, TT30, Ursa, ursodiol, Vangiolux, VAR10200, vascular endothelial growth factor antibody, vascular endothelial growth factor B, vascular endothelial growth factor kinoid, vascular endothelial growth factor oligonucleotide, VAST Compounds, vatalanib, VEGF antagonist (e.g., as described herein), verteporfm, Visudyne, Visudyne with Lucentis and dexamethasone, Visudyne with triamcinolone acetonide, Vivis, volociximab, Votrient, XV615, zeaxanthin, ZFP TF, zinc-monocysteine and Zybrestat. In one embodiment, one or more of the macular degeneration treating drugs described above is combined with one or more agents listed above or herein or with other agents known in the art.

In one embodiment, the drug delivered to the SCS using the non-surgical methods described herein is an antagonist of a member of the platelet derived growth factor (PDGF) family, for example, a drug that inhibits, reduces or modulates the signaling and/or activity of PDGF-receptors (PDGFR). For example, the PDGF antagonist delivered to the suprachoroidal space for the treatment of one or more posterior ocular disorders or choroidal maladies, in one embodiment, is an anti-PDGF aptamer, an anti-PDGF antibody or fragment thereof an anti-PDGFR antibody or fragment thereof or a small molecule antagonist. In one embodiment, the PDGF antagonist is an antagonist of the PDGFRa or PDGFRp. In one embodiment, the PDGF antagonist is the anti-PDGF-β aptamer E10030, sunitnib, axitinib, sorefenib, imatinib, imatinib mesylate, nintedanib, pazopanib HCl, ponatinib, MK-2461, Dovitinib, pazopanib, crenolanib, PP-121, telatinib, KRN 633, CP 673451, TSU-68, Ki8751, amuvatinib, tivozanib, masitinib, motesanib diphosphate, dovitinib dilactic acid, linifanib (ABT-869). In one embodiment, the intraocular elimination half life (t_{1/2}) of the PDGF antagonist administered to the suprachoroidal space is greater than the intraocular t_{1/2} of the PDGF antagonist, when administered intravitreally, intracamerally, topically, parenterally or orally. In another embodiment, the mean intraocular maximum concentration (Cₘₐₓ) of the PDGF antagonist, when administered to the suprachoroidal space via the methods described herein, is greater than the intraocular Cₘₐₓ of the PDGF antagonist, when administered intravitreally, intracamerally, topically, parenterally or orally. In another embodiment, the mean intraocular area under the curve (AUCₒ₋ₜ) of the PDGF antagonist, when administered to the suprachoroidal space via the methods described herein, is greater than the intraocular AUC₀₋ₜ of the PDGF antagonist, when administered intravitreally, intracamerally, topically, parenterally or orally.

In one embodiment, a drug that treats, prevents and/or ameliorates fibrosis is used in conjunction with the devices and methods described herein and is delivered to the SCS of the eye. In a further embodiment, the drug is interferon gamma 1b (Actimmune®) with pirfenidone, ACUHTR028, AlphaVBetaS, aminobenzoate potassium, amyloid P, ANG1122, ANG1170, ANG3062, ANG3281, ANG3298, ANG4011, Anti-CTGF RNAi, Aplidin, astragalus membranaceus extract with salvia and schisandra chinensis, atherosclerotic plaque blocker, Azof, AZX100, BB3, connective tissue growth factor antibody, CT140, danazol, Esbriet, EXC001, EXC002, EXC003, EXC004, EXC005, F647, FG3019, Fibrocorin, Follistatin, FT011, Galectin-3 inhibitors, GKT137831, GMCT01, GMCT02, GRMD01, GRMD02, GRN510, Heberon Alfa R, interferon alfa-2b, interferon gamma-1b with pirfenidone, ITMN520, JKB 119, JKB121, JKB122, KRX168, LPA1 receptor antagonist, MGN4220, MIA2, microRNA 29a oligonucleotide, MMI0100, noscapine, PBI4050, PBI4419, PDGFR inhibitor, PF-06473871, PGN0052, Pirespa, Pirfenex, pirfenidone, plitidepsin, PRM151, Px102, PYN17, PYN22 with PYN17, Relivergen, rhPTX2 Fusion Proteins, RXI109, secretin, STX100, TGF-beta Inhibitor, transforming growth factor, beta receptor 2 oligonucleotide, VA999260 or XV615. In one embodiment, one or more of the fibrosis treating drugs described above is combined with one or more agents listed above or herein or with other agents known in the art.

In one embodiment, a drug that treats, prevents and/or ameliorates diabetic macular edema is used in conjunction with the devices and methods described herein and is delivered to the SCS of the eye. In a further embodiment, the drug is AKB9778, bevasiranib sodium, Candy, choline fenofibrate, Cortiject, c-raf 2-methoxyethyl phosphorothioate oligonucleotide, DE109, dexamethasone, DNA damage inducible transcript 4 oligonucleotide, FOV2304, iCo007, KH902, MP0112, NCX434, Optina, Ozurdex, PF4523655, SAR1118, sirolimus, SK0503 or TriLipix. In one embodiment, one or more of the diabetic macular edema treating drugs described above is combined with one or more agents listed above or herein or with other agents known in the art.

In one embodiment, a drug that treats, prevents and/or ameliorates macular edema is used in conjunction with the devices and methods described herein and is delivered to the SCS of the eye. In a further embodiment, the drug is delivered to the SCS of a human subject in need of treatment of a posterior ocular disorder or choroidal malady via a hollow microneedle. In one embodiment, the drug is denufosol tetrasodium, dexamethasone, ecallantide, pegaptanib sodium, ranibizumab or triamcinolone. In addition, the drugs delivered to ocular tissues using the microneedle devices and methods disclosed herein which treat, prevent, and/or ameliorate macular edema, as listed above, may be combined with one or more agents listed above or herein or with other agents known in the art.

In one embodiment, a drug that treats, prevents and/or ameliorates ocular hypertension is used in conjunction with the devices and methods described herein and is delivered to the SCS of the eye. In a further embodiment, the drug is 2-MeS-beta gamma-CC12-ATP, Aceta Diazol, acetazolamide, Aristomol, Arteoptic, AZD4017, Betalmic, betaxolol hydrochloride, Betimol, Betoptic S, Brimodin, Brimonal, brimonidine, brimonidine tartrate, Brinidin, Calte, carteolol hydrochloride, Cosopt, CS088, DE092, DE104, DE111, dorzolamide, dorzolamide hydrochloride, Dorzolamide hydrochloride with Timolol maleate, Droptimol, Fortinol, Glaumol, Hypadil, Ismotic, isopropyl unoprostone, isosorbide, Latalux, latanoprost, Latanoprost with Timolol maleate, levobunolol hydrochloride, Lotensin, Mannigen, mannitol, metipranolol, mifepristone, Mikelan, Minims Metipranolol, Mirol, nipradilol, Nor Tenz, Ocupress, olmesartan, Ophtalol, pilocarpine nitrate, Piobaj, Rescula, RU486, Rysmon TG, SAD448, Saflutan, Shemol, Taflotan, tafluprost, tafluprost with timolol, Thiaboot, Timocomod, timolol, Timolol Actavis, timolol hemihydrate, timolol maleate, Travast, travoprost, Unilat, Xalacom, Xalatan or Zomilol. In addition, the drugs delivered to the SCS using the microneedle devices and methods described herein which treat, prevent, and/or ameliorate ocular hypertension, as listed above, may be combined with one or more agents listed above or herein or with other agents known in the art.

Microneedle Devices

The microneedle devices used for administration of the formulations provided herein include one or more microneedles. The microneedles may be hollow (e.g., where a fluid drug formulation is infused through the microneedle bore) or solid (e.g., where the drug formulation is coated onto the microneedle). The device also may include an elongated housing for holding the proximal end of the microneedle.

As used herein, the term “microneedle” refers to a structure having a base, a shaft, and a tip end suitable for insertion into the ocular tissue and has dimensions suitable for minimally invasive insertion and administration of the formulations described herein. That is, the microneedle has a length or effective length that from about 50 μm to about 2000 microns and a width (or diameter) from about 100 μm to about 500 μm.

In various embodiments, the microneedle may have a length of from about 50 μm, about 75 μm, about 100 μm, about 200 μm, about 300 μm, about 400 μm, or about 500 μm up to about 1500 μm, about 1250 μm, about 1000, about 999 μm, about 900 μm, about 800 μm, about 700 μm, about 600 μm, or about 500 μm. For example, in embodiments the microneedle may have a length from about 75 μm to about 1500 μm, about 200 μm to about 1250 μm, or about 500 μm to about 1000 μm.

In various embodiments, the proximal portion of the microneedle (i.e., the portion nearest its base) may have a width or cross-sectional dimension of from about 100 μm, about 150 μm, or about 200 μm up to about 500 μm, about 400 μm, about 350 μm, about 300 μm, about 250 μm, or about 200 μm. For example, in embodiments the microneedle may have a width at its base from about 100 μm to about 400 μm, from about 150 μm to about 400 μm, from about 200 μm to about 300 μm, or from about 250 μm to about 400 μm.

In embodiments, the tip end of the microneedle may have a planar or curved bevel. For example, a curved bevel may have a radius of curvature at its tip that is specially configured for the type of tissue that is being targeted. In one aspect, the tip end of the microneedle may have a radius of curvature at its tip of from about 100 nm to about 50 μm. For example, the tip end of the microneedle may have a radius of curvature at its tip of from about 200 nm, about 500 nm, about 1000 nm, about 2000 nm, about 5000 nm, or about 10,000 nm up to about 40 μm, about 30 μm, about 20 μm, or about 10,000 nm.

In embodiments, the microneedle extends from a base that may be integral with or separate from the microneedle. The base may be rigid or flexible and substantially planar or curved. For example, the base may be shaped to minimize contact between the base and the ocular tissue at the point of insertion and/or so as to counteract the deflection of the ocular tissue and facilitate insertion of the microneedle into the ocular tissue (e.g., extending toward the tip portion of the microneedle so as to “pinch” the ocular tissue).

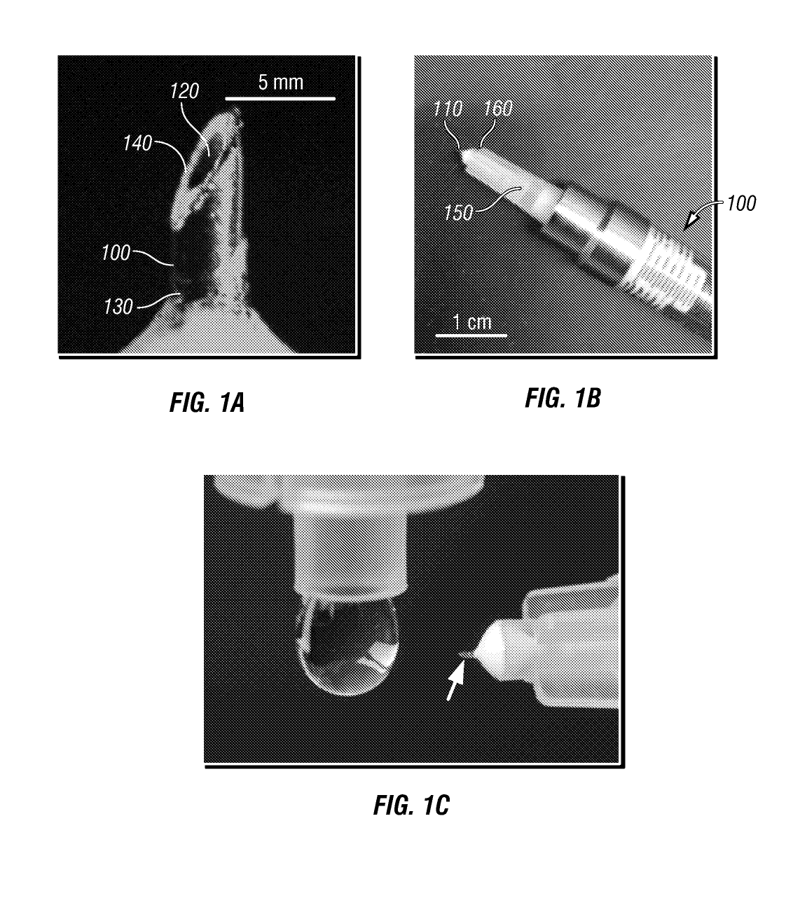

An exemplary microneedle device is illustrated in FIG. 1, which shows a microneedle device with a single hollow microneedle. As used herein, the term “hollow” includes a single straight bore through the center of the microneedle, as well as multiple bores, bores that follow complex paths through the microneedles, multiple entry and exit points from the bore(s), and intersecting or networks of bores. That is, a hollow microneedle has a structure that includes one or more continuous pathways from the base of the microneedle to an exit in the shaft and/or tip portion of the microneedle distal to the base. In such embodiments, the device may further include a means for conducting a fluid formulation through the hollow microneedle. For example, the means may be a flexible or rigid conduit in fluid connection with the base or proximal end of the microneedle. The means may also include a pump or other devices for creating a pressure gradient for inducing fluid flow through the device. The conduit may be in operable connection with a source of the fluid formulation. For example, the source may be any suitable container, such as a conventional syringe or a disposable unit dose container.

The exemplary microneedle device 100 illustrated in FIGS. 1A and 1B includes a hollow microneedle 110 having a hollow bore 120 through which a fluid formulation can be delivered to the eye or through which a biological fluid can be withdrawn from the eye. The microneedle 110 includes a proximal portion 130 and a tip portion 140 extending from a base (not shown) secured in an adaptor 150. The adaptor 150 may comprise an elongated body having a distal end 160 from which the proximal portion 130 and tip portion 140 of the microneedle 110 extends, and may further comprise a means for securing the base portion of the microneedle 110 within the distal end 160 of the adaptor 150 (e.g., a screw or pin). In some embodiments, the microneedle device may be adjustable such that the proximal portion and tip portion of the microneedle extending from the adaptor may be adjusted depending on the depth of the ocular tissue at the insertion site.

The microneedle device may further include a fluid reservoir for containing the fluid drug formulation, the fluid drug formulation being in operable communication with the bore of the microneedle at a location distal to the tip end of the microneedle. The fluid reservoir may be integral with the microneedle, integral with the adaptor, or separate from both the microneedle and adaptor.

In embodiments, the microneedle device may include an assembly or array of two or more microneedles. For example, the device may include an array of between two and 100 microneedles (e.g., any number from two, three, five, 10, 20, and 50). In embodiments, the array of microneedles may include a combination of different microneedles. For instance, the array may include microneedles of various lengths, base portion diameters, tip portion shapes, spacings, coatings, and the like.

The microneedles can be formed/constructed of different biocompatible materials, including metals, glasses, semi-conductor materials, ceramics, or polymers. Exemplary metals include pharmaceutical grade stainless steel, gold, titanium, nickel, iron, gold, tin, chromium, copper, and alloys thereof. Exemplary polymers may be biodegradable or non-biodegradable. Non-limiting examples of biodegradable polymers include polylactides, polyglycolides, polylactide-co-glycolides (PLGA), polyanhydrides, polyorthoesters, polyetheresters, polycaprolactones, polyesteramides, poly(butyric acid), poly(valeric acid), polyurethanes and copolymers and blends thereof. Non-limiting examples of non-biodegradable polymers include various thermoplastics or other polymeric structural materials known in the fabrication of medical devices, such as nylons, polyesters, polycarbonates, polyacrylates, polymers of ethylene-vinyl-acetates and other acyl substituted cellulose acetates, non-degradable polyurethanes, polystyrenes, polyvinyl chloride, polyvinyl fluoride, poly(vinyl imidazole), chlorosulphonate polyolefins, polyethylene oxide, and blends and copolymers thereof. Biodegradable microneedles may be beneficial by providing an increased level of safety as compared to non-biodegradable ones, such that the microneedles are essentially harmless even if inadvertantly broken off into the ocular tissue or are rendered unsuitable for use.

The microneedle can be fabricated by a variety of methods known in the art or as described in the examples. In one embodiment, the microneedle is fabricated using a laser or similar optical energy source. For example, a hollow microneedle may be fabricated from a microcannula cut using a laser to the desired microneedle length. The laser may also be used to shape single or multiple tip openings for hollow microneedles. Single or multiple cuts may be performed on a single microcannula to shape the desired microneedle structure (e.g., to obtain the desired radius of curvature at the microneedle tip). In one example, the microcannula may be made of metal such as stainless steel and cut using a laser with a wavelength in the infrared region of the light spectrum (0.7-300 μm). Further refinement may be performed using metal electropolishing techniques familiar to those in the field. In another embodiment, the microneedle length and optional bevel shape is formed by a physical grinding process, which for example, may include grinding a metal cannula against a moving abrasive surface. The fabrication process may further include precision grinding, micro-bead jet blasting and ultrasonic cleaning to form the shape of the desired precision tip of the microneedle.

Further details of possible manufacturing techniques are described, for example, in PCT Publication No. WO 2014/036009, U.S. Patent Application Publication No. 2006/0086689 to Raju et al., U.S. Patent Application Publication No. 2006/0084942 to Kim et al., U.S. Patent Application Publication No. 2005/0209565 to Yuzhakov et al., U.S. Patent Application Publication No. 2002/0082543 to Park et al., U.S. Pat. No. 6,334,856 to Allen et al., U.S. Pat. No. 6,611,707 to Prausnitz et al., or U.S. Pat. No. 6,743,211 to Prausnitz et al.

Delivering drugs to the eye can be challenging due to complex anatomy and unique physiology of the eye. Thus, in order to treat ophthalmic diseases effectively, both the effectiveness of the drug and the delivery method may be carefully considered in view of the complex ocular anatomy that can prevent penetration of the drug to the targeted location and reduce the efficiency of the pharmacotherapies. The embodiments of formulations, systems, and methods for administration provided herein advantageously overcome these difficulties by enhancing targeting of pharmacotherapies to specific ocular tissues, such as the cornea, ciliary body, choroid, and posterior segment of the eye, using microneedles as a drug delivery platform. The embodied formulations enable highly targeted administration of formulations, and provide many advantages not capable of being attained using existing, prior art formulations. For example, (i) bioavailability may approach 100% by delivering drugs directly to the targeted tissue, (ii) side effects may be reduced due to administration of a lower dosage that is enabled by delivering more drugs to the targeted site, and (iii) patient compliance can be improved by administering longer controlled-release formulations that would not be possible without highly targeted delivery.

Embodiments of the present invention may be further understood with reference to the following non-limiting examples.

The following examples illustrate the various advantages and features of the present description. Example 1 summarizes a study of targeted delivery of protein therapeutics into the cornea using coated microneedles to suppress corneal neovascularization in an injury-induced rabbit model. The results showed that minimally invasive administration of a protein therapeutic (bevacizumab) locally into the intracorneal space of the cornea that was effective to suppress neovascularization using a much lower dose than other conventionally used methods. Example 2 summarizes a study of targeted delivery to the ciliary body and choroid via suprachoroidal space injection using novel polymeric excipient formulations that immobilized injected polymeric particles to target ciliary body or enhanced mobility of polymeric particles to target the entire layer of the choroid. The results showed that a strongly non-Newtonian fluid was effective to immobilize the particles at the injection site up to 2 months as compared to the high molecular weight formulation with weakly non-Newtonian fluid that was effective to increase the spreading of particles away from the injection site to provide 100% coverage of the choroidal surface with a single injection. The results also demonstrated that significant dose sparing (on the order of 500-1000-fold) was attainable by targeted delivery via supraciliary space injection. Example 3 summarizes a study of novel emulsion droplets to target different locations within the eye using gravity-mediated delivery technique via suprachoroidal space injection. The results showed that particle-stabilized emulsion droplets of a high-density emulsion were effective to create movement inside the suprachoroidal space in the direction of gravity. Example 4 summarizes a study of formulations developed either to immobilize particles at the site of injection or to enhance the spreading of the particles throughout the suprachoroidal space. The results showed that particles up to 10 μm in size could be targeted to the ciliary body or throughout the choroid using non-Newtonian formulations of polymers having different viscosity, molecular weight and hydrophobicity.

### EXAMPLE 1

Corneal neovascularization is the invasion of blood vessels into the clear cornea, which can cause visual impairment. Conventional therapy for corneal neovascularization relies on steroids, such as hydrocortisone and dexamethasone; however, steroids carry the risk of serious side effects such as cataract and glaucoma. Recently, anti-vascular endothelial growth factor (VEGF) treatments have shown promising results for treating corneal neovascularization. Currently, topical and subconjunctival injection of bevacizumab is used off-label in clinic to treat corneal neovascularization; however, topical administration is extremely inefficient due to the barrier properties of corneal epithelium, and systemic delivery is often accompanied by side effects. Subconjunctival administration is a more efficient and targeted delivery method; however, subconjunctival injection of bevacizumab can cause side effects due to the high dose requirements and may not be suitable for long-term use. Intrastromal injection of bevacizumab using a hypodermic needle has recently shown promising results. Thus, a study was conducted to assess the efficacy of intrastromal delivery using microneedles in an injury-induced neovascularization model and compared microneedle-based therapy to conventional topical and subconjunctival delivery of bevacizumab.

Fluorescent Labeling of Bevacizumab

Bevacizumab (Avastin, Genentech, South San Francisco, Calif.) was labeled using a SAIVI Alexa Fluor 750 Antibody/Protein labeling kit protocol (Invitrogen-Molecular Probes, Eugene, Oreg.). Briefly, Alexa Fluor NHS esters were incubated with the protein in a basic medium (pH 9.3). Labeled protein (bevacizumab) was isolated and purified by gel filtration. The final dye-to-protein ratio (number of Alexa Fluor molecules coupled to each protein molecule) was determined to be between 2.5 and 3.5 according to a protocol from Invitrogen. Finally, this solution of labeled protein (8 mg/mL) was mixed with untagged bevacizumab (i.e., Avastin, 25 mg/mL) at a volumetric ratio of 1:1 and was stored in the dark at 4° C.

Enzyme-Linked Immunosorbent Assay (ELISA) of Bevacizumab

A serial dilution of bevacizumab (6.25-50 ng/mL) was used to generate a standard curve. Bevacizumab-coated microneedles were dissolved in phosphate-buffered saline (PBS) and diluted as needed to bring the concentration into the ELISA assay range. Diluted solutions were put in triplicate into wells in a Maxisorp ELISA plate (Nunc, Roskilde, Denmark). Plates with vascular endothelial growth factor (VEGF-165, R&D Systems, Minneapolis, Minn.) were coated overnight at 4° C. in sodium carbonate buffer at pH 9.6. Plates were washed three times with PBS-T (PBS with 0.05% Tween-20) and blocked with 300 μL per well of 1% bovine serum albumin (BSA) in PBS for 2 hours at room temperature. After three washes with 300 μL PBS-T each, 100 μL of bevacizumab-containing samples were added in triplicate for 2 hours at room temperature. They were then washed three times with PBS-T as above and 100 μL horseradish peroxidase-labeled goat-anti-human IgG (R&D Systems) in 0.1% BSA per well and then incubated for 2 hours at room temperature. Washing was performed as described and 100 μL of TMB (3,3′,5,5″-tetramethylbenzidine) substrate reagent solution (R&D Systems) was transferred into each well. Reaction was terminated after 20 min by adding 50 μL of 0.5 M HCl to each well. Absorbance was measured spectrophotometrically at a wavelength of 450 nm (iMark Microplate Reader, Bio-Rad, Hercules, Calif.).

Microneedle Fabrication and Coating

To make coating formulations, the solution described above containing a mixture of labeled and unlabeled bevacizumab was further diluted with stock solution of bevacizumab (i.e., Avastin, 25 mg/mL) at a volumetric ratio of 1:1. The mixed solution was repeatedly centrifuged using Nanosep centrifuge filters (Port Washington, N.Y.) with a 3 kDa molecular weight cutoff until the retentate reached a concentration of 100 mg/mL of bevacizumab. This solution was then immediately mixed with 5% carboxymethylcellulose at a volumetric ratio of 1:3 to make the final coating solution.

Solid microneedles were fabricated by cutting needle structures from stainless steel sheets (SS304, 75 μm thick; McMaster Carr, Atlanta, Ga.) using an infrared laser (Resonetics Maestro, Nashua, N.H.) and then electropolished to yield microneedles of defined geometry that were 400 μm in length, 150 μm in width, 75 μm in thickness, and 55° in tip angle. Prior to coating, microneedles were treated in a plasma cleaner (PDC-32CG, Harrick Plasma, Ithaca, N.Y.) to facilitate coating of the formulation on the micronneedles. Microneedles were coated by dipping 10 to 40 times into the coating solution at room temperature.

Hollow microneedles were fabricated from borosilicate micropipette tubes (Sutter Instrument, Novato, Calif.). A custom, pen-like device with a threaded cap was fabricated to position the microneedle and allow precise adjustment of its length. This device was attached to a gas-tight, 10-μL glass syringe (Thermo Scientific, Waltham, Mass.).

Induction of Corneal Neovascularization

All animal studies adhered to the ARVO statement for the Use of Animals in Ophthalmic and Vision Research and were approved by the Georgia Institute of Technology Institutional Animal Care and Use Committee (IACUC). Male and female New Zealand rabbits (2.2-2.5 kg) were anesthetized with ketamine (17 mg/kg), xylazine (8.5 mg/kg) and acepromazine (0.5 mg/kg) subcutaneously. Following topical administration of 0.5% proparacaine hydrochloride to minimize discomfort, a single 7.0-gauge silk suture (Ethicon TG140, Blue Ash, Ohio) was placed at midstromal depth 1 mm away from the limbus of the rabbit cornea to generate corneal neovascularization associated with minor traumatic injury. This silk suture was left in the rabbit cornea for the duration of the experiment to induce neovascularization. For each animal, a suture was placed in one eye and the companion eye was left untreated.

Measurement of Neovascularization

During the experiment, the rabbit eye was imaged using a digital camera (Cannon Rebel Tli, Melvile, N.Y.) with macroscopic lens (Cannon MP-E 65 mm) at 3× magnification every two days after placement of the suture. The area of neovascularization was quantified using Adobe Photoshop (Adobe, Jan Jose, Calif.).

Experimental Treatment Groups

Prior to all treatment procedures except for topical delivery, rabbits were anesthetized with ketamine (6 mg/kg), xylazine (4 mg/kg) and acepromazine (0.25 mg/kg) subcutaneously. A reduced dose of anesthesic compared to the suture insertion procedure was used to reduce possible stress to the animals. A single drop of topical proparacaine ophthalmic solution was given as anesthesia. The duration of each study was 18 days and, after the suture insertion at the beginning of the experiment, 4 days were allowed for neovascularization to develop. All the treatments were done on day 4 except as indicated below. The treatment groups are listed in the table below.

Treatment groups

| | |
|---|---|
| UT | Untreated group |
| TOP | Topical delivery group |
| SC-high | High-dose subconjunctival group |
| SC-low | Low-dose subconjunctival group |
| MN-1bolus | 1 microneedle bolus delivery group |
| MN-4bolus | 4 microneedle bolus delivery group |
| MN-1 × 3 | 1 microneedle, 3 doses delivery group |
| MN-placebo | 1 microneedle placebo group |
| MN-hollow | Hollow microneedle bolus delivery group |

Untreated Group (UT)

Other than applying a suture to the eye, these animals received no further treatments.

Topical Delivery Group (TOP)

Topical delivery of bevacizumab was given into the upper conjunctival sack without anesthesia three times per day (at approximately noon, 3:00 μm and 6:00 μm) on day 4 through day 17. Each drop contained 1250 μg of bevacizumab in 50 μL, for a daily dose of 3750 μg of bevacizumab and a total dose of 52,500 μg of bevacizumab over the course of 14 days of treatment.

Subconjunctival Delivery Groups (SC)

Bevacizumab was injected subconjunctivally with a 30-gauge hypodermic needle at the upper bulbar conjunctiva four days after suture placement. The high-dose group (SC-high) received 2500 μg of bevacuzumab (in 100 μL, i.e., Avastin). The low-dose group (SC-low) received 4.4 μg of bevacuzumab (Avastin was diluted with HBSS to 100 μL).

Microneedle Delivery Groups (MN)

Microneedles designed to deliver 1.1 μg of bevacizumab were inserted at the site of silk suture placement in the cornea and left in place for 1 min to allow dissolution of the coating. For the one-microneedle bolus delivery group (MN-1bolus), a single microneedle (i.e., 1.1 μg of bevacizumab) was given as a bolus dose four days after suture placement. For the four-microneedle bolus delivery group (MN-4bolus), four microneedles (i.e., 4.4 μg of bevacizumab) were given as a bolus dose four days after suture placement. For the one-microneedle three doses delivery group (MN-1×3), a single microneedle (i.e., 1.1 μg of bevacizumab) was given as at 4, 6 and 8 days after suture placement (i.e., for a total dose of 3.3 μg of bevacizumab). For the microneedle placebo group (MN-placebo), four microneedles coated with formulation containing no bevacizumab was given as a bolus dose four days after suture placement. Finally, for the hollow microneedle bolus delivery group (MN-hollow), a hollow microneedle was used to inject 2 μL of 25 mg/mL bevacizumab (i.e., Avastin, dose of 50 μg bevacizumab) intrastromally at the site of silk suture placement as a bolus dose four days after suture placement. After all of the insertion procedures, the eyelid was left closed for 5 min, after which all the tear fluid was wiped off the eye to collect any residual bevacizumab that was not able to penetrate into the cornea using a small piece of a Kimwipe towel. The used towels and microneedles were collected and incubated in HBSS to collect residual bevacizumab.

Fluorescently Labeled Bevacizumab Imaging Study

Prior to imaging, rabbits were anesthetized by subcutaneous injection using ketamine/xylazine/acepromazine at concentrations of 6/4/0.25 mg/kg. Eyes were kept open using a lid speculum for the duration of the imaging procedures. The fluorescence signal intensity in the rabbits was imaged on a In Vivo Imaging System (IVIS; Caliper Xenogen Lumina, Waltham, Mass.) at 0, 2, and 4 days post-insertion. Animals were imaged at 745 nm excitation wavelength, 780 nm emission wavelength and 1 sec exposure time. Fluorescence intensity was measured as background-subtracted average efficiency within a fixed region of interest centered on the insertion site.

Safety Study

To identify possible microanatomical changes after intrastromal delivery using microneedles, we conducted a histological safety study using four study groups: (i) The untreated group received no suture and no other treatments. (ii) The suture-only group received a suture at day 0, but no other treatments. (iii). The suture with non-coated microneedles group received a suture on day 0 and four non-coated microneedles inserted at the site of the suture on day 4. (iv) The suture with coated microneedles group received a suture on day 0 and four microneedles each coated with 1.1 μg of bevacizumab inserted at the site of the suture on day 4. Animals were sacrificed on days 1, 6, 10 and/or 18 for histological analysis. Suture placement and microneedle application were carried out as described above. High magnification images were taken every day in all study groups to assess possible gross corneal damage. Corneal tissues were fixed in 10% formalin and embedded in paraffin. Hematoxylin-eosin (HE) or periodic acid-Schiff (PAS) staining was performed.

Statistical Analysis

Replicate pharmacodynamics experiments were done for each treatment group above. The mean and standard error of mean were calculated from multiple (3-6) images. Experimental data were analyzed using two-way analysis of variance (ANOVA) to examine the difference between treatments. In all cases, a value of p<0.05 was considered statistically significant.

Characterization of Microneedles Coated with Bevacizumab

Solid microneedles were first designed to penetrate into, but not across, the cornea and in that way deposit drug coated onto the microneedles within the corneal stroma at the site of microneedle penetration. Guided by the average rabbit corneal thickness of 400 μm and possible tissue deformation during microneedle insertion, the microneedles used for rabbit corneal insertion were 400 μm in length, 150 μm in width, 75 μm in thickness, and 55° in tip angle. These microneedles were coated with a dry film of bevacizumab that was localized to the microneedle shaft and not on the supporting base structure. Coatings were applied by dipping repeatedly into a solution of bevacizumab using an automated coating machine. This design enabled efficient delivery of bevacizumab into the corneal stroma at the site of microneedle insertion (data not shown).

Intracorneal/Intrastromal Delivery of Bevacizumab In Vivo

In vivo bioavailability of bevacizumab delivered from coated microneedles was quantified by tagging the bevacizumab with florescent dye. Alexa Fluor 750 dye was tagged to bevacizumab to quantify using ELISA. Microneedles prepared by coating with 10, 20, 30 or 40 dips were inserted into the cornea of an anesthetized rabbit. The amount of bevacizumab coated per microneedle was quantified using ELISA. Coated microneedles were inserted into but not across the cornea for 60 sec and then removed. The insertion time of 60 sec was used as it was expected it to be sufficient to dissolve most of the coating off the microneedles while minimizing possible patient discomfort and clinical throughput time in future applications. Images showed that the bevacizumab coating was largely deposited in the corneal stroma.

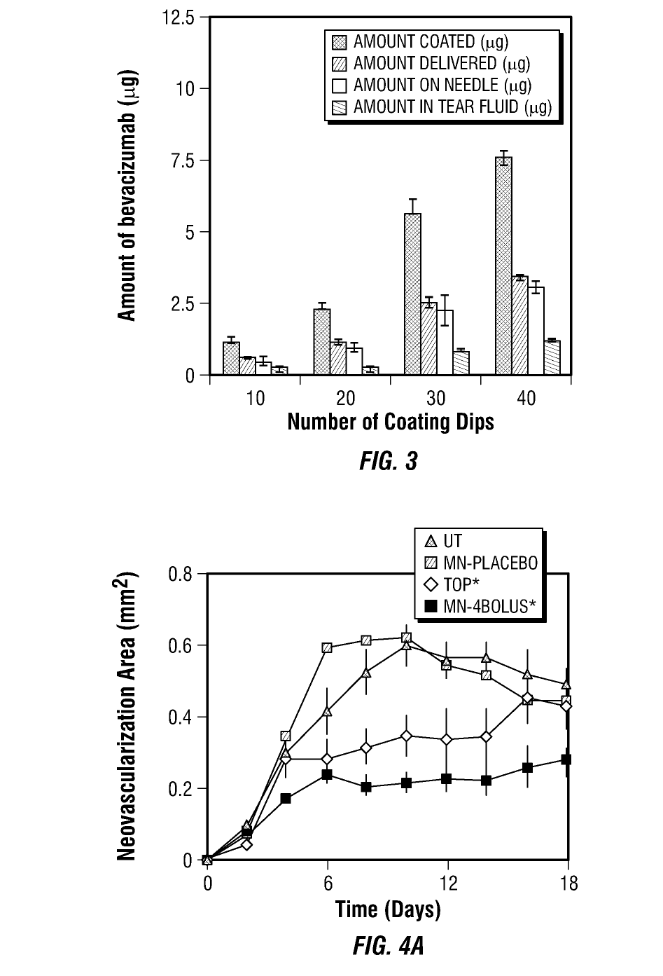

The amount of bevacizumab coated onto microneedles increased linearly from 1.1 μg to 7.6 μg per microneedles with increasing number of dip coats (FIG. 3). However, the amount of bevacizumab delivered into the cornea increased linearly with coating amount. For example, coatings produced using 10 dip coats delivered 52% of the coated bevacizumab into the cornea, with most of the remaining drug still coated on the microneedle, whereas coatings produces using 40 dip coated delivered just 44% of the coated drug. These delivery efficiencies are similar to results from a previous study using fluorescein-coated microneedles in rabbit eyes. This effect may be explained by thick coatings on microneedles making insertion into tissue and rapid dissolution in the tissue more difficult. Given these data, microneedles coated with 20 dips were selected as a compromise formulation that can deliver 1.14±0.11 μg of bevacizumab with reasonable efficiency for the pharmacodynamic tests in this study.

Efficacy of Intrastromal Delivery of Bevacizumab

Using Microneedles Compared to Topical Delivery

To further assess the capability of microneedles as an intrastromal drug delivery platform, injury-induced neovascularization was created in a rabbit model and bevacizumab was delivered using either microneedle or topical eye drops.

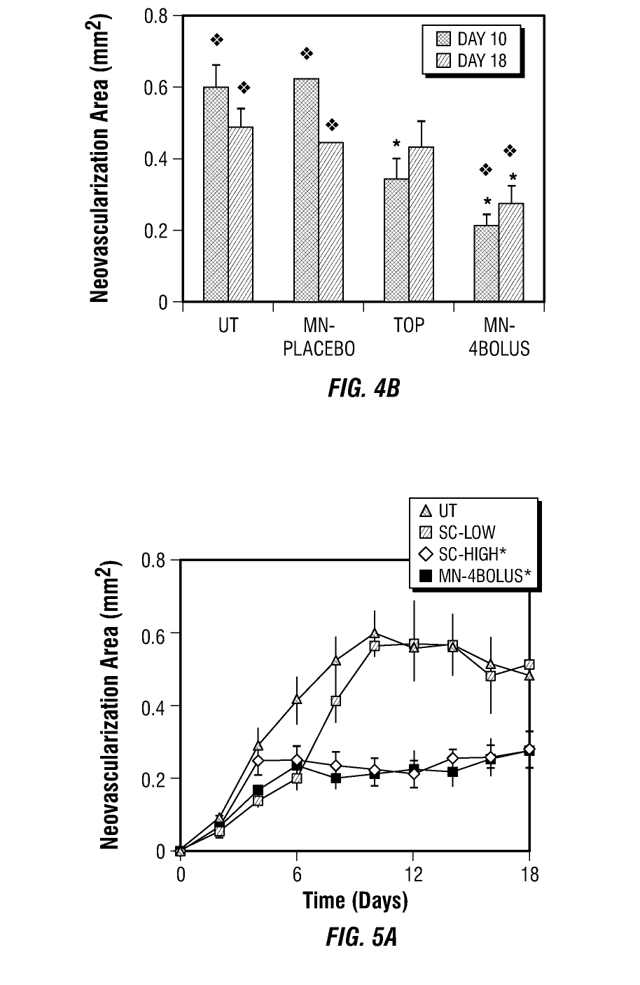

A suture was inserted into the mid-space of the cornea. All treatments were then started after 4 days, once significant neovascularization had developed. Changes in vascularization area in the eyes was measured using image analysis to compare the pharmacodynamics of topical and microneedle delivery. As negative controls, a group of rabbits were left untreated (UT) and another group of rabbits were treated with four placebo microneedles (MN-placebo; coated with drug-free formulation). The untreated and placebo microneedle groups showed similar changes in corneal neovascularization with no statistical difference (p=0.11), where the neovascularization area increased until day 10 and then decreased slightly until day 18 (FIGS. 4A and 4B). The peak neovascularization area for the untreated group was 0.60±0.06 mm² on day 10 and by day 18 area was 0.49±0.05 mm² (FIGS. 4A and 4B).

For the topical delivery group (TOP), 3 topical eye drops were given every day from day 4 through the end of the experiment (day 18), which is a total of 52,500 delivered g of bevacizumab over a period of 14 days (i.e., 3750 delivered g/day). Topical eye drops reduced neovascularization compared to the untreated eyes by 44% (day 10) and 6% (day 18) (FIGS. 4A and 4B). The topical eye drops group showed an immediate inhibition of the blood vessel growth after starting the treatment at day 4. However, neovascularization area increased steadily after that until the end of the experiment. At day 18, the topical eye drops group showed no significant difference versus the untreated eyes (one-way ANOVA, p=0.36). Two-way ANOVA analysis showed that the change in neovasculaturization area for the topical group over time was significantly different from the untreated group (p<0.0001). This was consistent with literature data that topical administration of bevacizumab can reduce corneal neovascularization.

For the microneedles group (MN-4bolus), eyes were treated one time with 4.4 delivered g of bevacizumab using four microneedles. This small dose administered using microneedles reduced neovascularization area compared to the untreated eyes by 65% (day 10) and 44% (day 18) (FIGS. 4A and 4B). Two-way ANOVA analysis showed that the microneedles group was significantly more effective at reducing corneal neovascularization compared to the untreated group (p<0.0001) and the topical group (p<0.0001), even though the microneedles group used 9722 times less bevacizumab compared to topical delivery.

The fact that intrastromal delivery of just 4.4 delivered g of drug using microneedles outperformed the administration of 52,500 delivered g of topical bevacizumab showed the inefficiency of the topical delivery and the highly targeted nature of intrastromal delivery (data not shown). This low bioavailability of bevacizumab by topical delivery can be explained by the strong barrier properties of the cornea to macromolecules and the rapid clearance of topical formulations from the precorneal space. In possible future clinical use, the dose sparing enabled by intrastromal delivery may reduce the risk of adverse events associated with prolonged topical administration of bevacizumab.

Efficacy of Intrastromal Delivery of Bevacizumab

Using Microneedles Compared to Subconjunctival Delivery

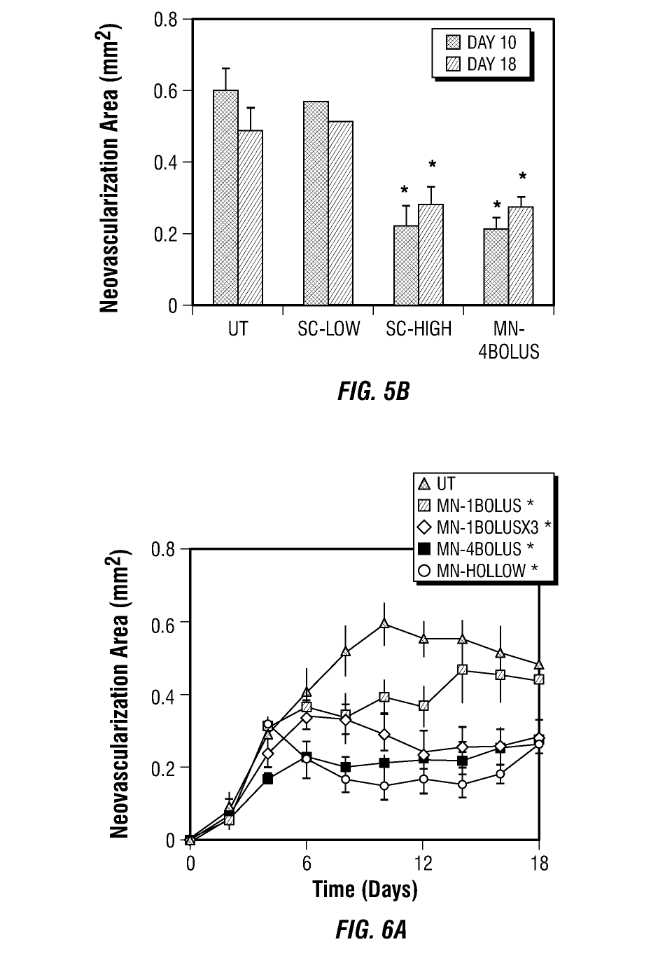

The pharmacodynamics of subconjunctival versus microneedle delivery methods were compared by measuring changes in neovascularization area in eyes treated with high-dose (SC-high) and low-dose (SC-low) subconjunctival injection of bevacizumab. Based on the reported effective dose in literature, 2500 μg (i.e., 100 μL of a 25 μg/μL bevacizumab solution) was given as a bolus on day 4 for the high-dose subconjunctival injection. For the low-dose subconjunctival injection, the microneedle dose that was able to inhibit neovascularization (see FIG. 5A) was matched. For this group, 4.4 μg of bevacizumab was given as a bolus on day 4.

Eyes treated with a low dose of 4.4 μg of bevacizumab by subconjunctival injection (SC-low) had no significant effect on neovascularization compared to the untreated eyes (UT) (FIG. 5A, two-way ANOVA, p=0.05). For the high-dose subconjunctival injection (SC-high), eyes treated with 2500 μg of bevacizumab significantly reduced neovascularization compared to the untreated eye by 62% (day 10) and 29% (day 18) (FIG. 5B, two-way ANOVA p<0.0001) and was not significantly different compared to the microneedles group (MN-4bolus) (FIG. 5B, two-way ANOVA, p=0.45). Although the pharmacodynamic responses for the microneedle group and high-dose subconjunctival group were similar, the microneedle group received 568 times less bevacizumab. This effect can be explained by the highly targeted nature of intrastromal delivery using microneedles.

Effect of Bevacizumab Dose on Efficacy Of Intrastromal

Delivery Using Microneedles

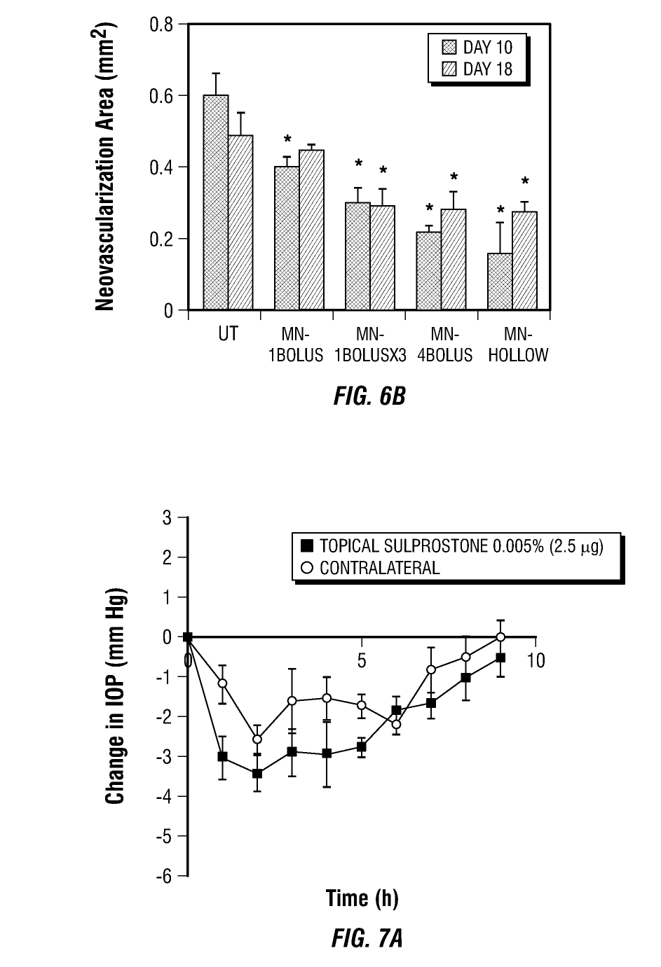

Other intrastromal doses were studied to improve the dosing regimen. First, a lower dose of 1.1 μg was given as a bolus on day 4 using a single microneedle (MN-1bolus). The average neovascularization area was 34% (day 10) and 10% (day 18) lower after this low-dose intrastromal bolus (MN-1bolus) compared to the no treatment group (UT) (FIGS. 6A and 6B, two-way ANOVA, p=0.001). However, the low-dose intrastromal bolus (MN-1bolus) was not as effective at reducing neovascularization compared to the higher-dose bolus microneedle group (MN-4bolus) (FIGS. 6A and 6B, two-way ANOVA, p<0.0009). This showed that an intrastromal bolus of 1.1 μg bevacizumab was effective, but a bolus of 4.4 μg bevacizumab was more effective.

Next, administration of bevacizumab as multiple sequential doses, in which eyes were treated with one microneedle administering 1.1 μg bevacizumab on days 4, 6 and 8 (i.e., for a total of 3.3 μg bevacizumab) was measured. This protocol (MN-1bolusx3) reduced neovascularization area by 50% (day 10) and 41% (day 18), which was significantly better compared to the untreated group (UT) (FIGS. 6A and 6B, two-way ANOVA, p<0.0009), but was not as effective as the bolus high-dose microneedle group (MN-4bolus) (FIGS. 6A and 6B, two-way ANOVA, p=0.019). The three-dose protocol (MN-1bolus×3) appeared to have a delayed effect on inhibiting neovascularization, where the first dose had only a partial effect, but after the third dose inhibition of neovascularization was equivalent to that achieved with the high-dose bolus (MN-4bolus). This showed that multiple small doses can be effective, but administration of a single bolus dose should be simpler in possible future clinical practice.

Finally, bolus intrastromal administration of an even higher dose of 50 μg of bevacizumab was measured. This high dose would have required the use of 46 coated microneedles, which is impractical. This larger dose was injected with a hollow microneedle (MN-hollow; 2 μL of a 25 μg/μL bevacizumab solution) and was found to reduce neovascularization compared to untreated eyes (UT) by 74% (day 10) and 45% (day 18), (FIGS. 6A and 6B, two-way ANOVA, p<0.0009) and was not significantly different compared to the bolus high-dose microneedle group (MN-4bolus) (FIGS. 6A and 6B, two-way ANOVA, p=0.154). This showed that giving a bolus dose more than 4.4 μg of bevacizumab did not provide additional improvement. However, this comparison was complicated by the fact that the high dose (MN-hollow) was given as a liquid solution that spread over a larger area in the corneal stroma, in contrast to the solid formulation (MN-4bolus) that dissolved off the solid microneedles at the sites of microneedle insertion.

Safety of Intrastromal Delivery of Bevacizumab

The rabbit corneas with and without microneedle treatment and with and without suture placement were evaluated to assess the safety of microneedle insertion by both magnified inspection of the corneal surface in vivo and histological examination of tissue sections obtained at various times after microneedle treatment Immediately after insertion and removal of the microneedle, a small puncture in the corneal epithelium was evident with a size on the order of 200 μm (data not shown). By the next day, it was not possible to locate the insertion site due to apparent repair of the epithelium. Similarly, at later times the corneal surface continued to look intact and normal. Eyes treated with bevacizumab-coated microneedles also were examined, and again showed only a microscopic puncture in the corneal epithelium that disappeared within one day and was not associated with any complications (data not shown). These injection sites were examined on a daily basis throughout the 18-day experiments, but no evidence of corneal opacity was observed in any of the 22 eyes treated with microneedles in this study.

In addition to examining the corneal surface, animals were sacrified at different time points to look for changes in corneal microanatomical structure. Histological analysis was carried out by an investigator who is board certified in both ophthalmology and anatomic pathology (data not shown). In comparison with untreated eyes, eyes treated by insertion of non-coated microneedles exhibited no significant changes in microanatomical structure of the cornea; no evidence of the corneal puncture could be found. There was also no significant presence of macrophages or vascularization observed. Histological sections from eyes that only had a suture applied were compared to an eye that had been sutured and then treated four days later with 1.1 μg bevacizumab using a microneedle. In the sutured eyes, there were large numbers of inflammatory cells present, but there were no notable differences seen between sutured eyes with and without microneedle treatment

### EXAMPLE 2

A study was conducted to assess the efficacy of supraciliary delivery using a hollow microneedle in the rabbit and to compare that to conventional topical delivery. This assessment was conducted by delivering anti-galucoma drugs to the supraciliary space and measuring reduction in intraocular pressure (TOP) over time compared to topical delivery of the same drugs. The drugs used in this study—sulprostone and brimonidine—both have sites of action in the ciliary body, which suggests that supraciliary targeting should be beneficial.

Sulprostone is a prostaglandin E2 analogue that has been shown to lower IOP in the rabbit, but is not used in humans to treat glaucoma. Latanoprost, travoprost, and bimatoprost are prostaglandin F2a analogues in common human clinical use, but rabbits respond poorly to these drugs. The receptors for prostaglandin analogues F2a are located in both trabecular meshwork and ciliary body in humans. The receptors for prostaglandin E2 analogues (e.g., sulprostone) are found in the ciliary body and iris of the rabbit. Although the mechanism of the action of prostaglandin E2 and F2a are different, the targeting or binding sites for both drugs are in the ciliary body. Therefore, sulprostone was used as a model analogue with a similar targeting site to other prostaglandin F2a analogues.

Brimonidine is in common clinical use for anti-glaucoma therapy and is active in the rabbit eye too.

Microneedle Fabrication and Formulation

Microneedles were fabricated from 33-gauge stainless steel needle cannulas (TSK Laboratories, Tochigi, Japan). The cannulas were shortened to approximately 700-800 μm in length and the bevel at the orifice was shaped using a laser (Resonetics Maestro, Nashua, N.H.), as described previously. The microneedles were electropolished using an E399 electropolisher (ESMA, South Holland, Ill.) and cleaned with deionized water. Sulprostone (Cayman Chemical, Ann Arbor, Mich.) and 0.15% brimonidine tartrate ophthalmic solution (Alphagan® P, Allergan, Irvine, Calif.) were diluted in Hank's Balanced Salt Solution (HBSS, Cellgro, Manassas, Va.). For topical delivery, the final concentration was 0.05 mg/mL sulprostone or 1.5 mg/mL brimonidine tartrate. For supraciliary injection, the solution was diluted to a range of drug concentrations and included 2% carboxymethylcellulose (CMC, 700 kDa molecular weight, Sigma-Aldrich, St. Louis, Mo.) to increase viscosity and thereby improve localization of the drug at the site of injection.

Anesthesia and Euthanasia

All studies used New Zealand White rabbits of mixed gender weighing between 3-4 kg (Charles River Breeding Laboratories, Wilmington, Mass.). All of the animals were treated according to the ARVO statement for the Use of Animals in Ophthalmic and Vision Research. For supraciliary injections and for application of topical eye drops, rabbits were anesthetized using 0.5-3.0% isoflurane, unless otherwise noted. The isoflurane percentage was slowly increased from 0.5% up to 2.5% or 3.0% for 15 min. To achieve longer-lasting anesthesia for the supraciliary and intravitreal safety studies measuring IOP immediately after injection, anesthesia was achieved using subcutaneous injection of a mixture of ketamine (25 mg/kg) and xylazine (2.5 mg/kg). This ketamine/xylazine dose was also used during initial studies screening suitable anesthetics for this study. For brimonidine treated eyes, proparacaine (a drop of 0.5% solution) was given 1-3 min before each injection to locally numb the ocular surface. Animals were euthanized with an injection of 150 mg/kg pentobarbital into the ear vein.

Pharmacodynamics Studies

For supraciliary injection, a microneedle was attached to a 50-100 μL gas-tight glass syringe containing either (i) a placebo formulation of BSS or (ii) a drug formulation containing a specified concentration of either sulprostone or brimonidine tartrate. The eyelid of the rabbit was pushed back and the microneedle was inserted into the sclera 3 mm posterior to the limbus in the superior temporal quadrant of the eye. A volume of 10 μL was injected within 5 sec and the microneedle was removed from the eye 15 sec later to reduce reflux of the injected formulation. Topical delivery of sulprostone and brimonidine was achieved by administering an eye drop into the upper conjunctival sack. IOP was measured hourly for 9 hours after drug administration, as described below. Each treatment involved application of just one dose of one drug either topically or by supraciliary injection in one eye. After a recovery period of at least 14 days, rabbits were used for additional experiments, alternating between the left and right eyes.

Safety Studies

Supraciliary injections of either 10 μL or 50 μL of BSS were performed as described above. Intravitreal injection was performed by inserting a 30-gauge hypodermic needle across the sclera 1.5 mm posterior to the limbus in the superior temporal quadrant of the eye. A volume of 50 μL HBSS was injected within 5 sec and the needle was removed from the eye 15 sec later to reduce reflux. IOP was measured periodically for 1 hour after injection, as described below.

Tonometer Calibration

The tonometer (TonoVet, icare, Vantaa, Finland) used for this study is calibrated for use in dogs and cats, and was therefore re-calibrated both in vivo (N=4) and ex vivo (N=3) for the rabbit eye. Ex vivo rabbit eyes were cannulated using a 25-gauge hypodermic needle (Becton Dickinson). The needle was inserted 2-3 mm posteriorly from the limbus and was connected to a reservoir containing balanced salt solution (BSS, Baxter, Deerfield, Ill.) elevated to a known height in order to create a controlled pressure inside the eye. The surface of the eye was wetted using saline solution periodically (every 2-3 min) to mimic the wetting of the cornea by the tear fluid. The final measurements were made after confirming stable IOP for 5 min. Data over a range of IOPs (7.3-22 mmHg) were collected and used to generate a calibration curve to correct values reported by the TonoVet device to the actual values of IOP in the eyes.

For the in vivo study, rabbits were anesthetized using a subcutaneous injection of a mixture of ketamine (25 mg/kg) and xylazine (2.5 mg/kg). Proparacaine (a drop of 0.5% solution) was given 1-3 min before cannulation to locally numb the ocular surface. IOP was controlled in a similar manner to the ex vivo experiments using an elevated BSS reservoir and a similar calibration curve was generated.

The in vivo and in vitro experiments yielded calibration curves of y=1.18x+1.82 (R²=0.98) and y=1.01x+3.08 (R²=1.00), respectively, where x=IOP reported by the TonoVet tonometer and y=water column pressure applied to the eye. The resulting calibration curves showed approximately linear relationships with similar slopes. The in vivo calibration curve was used for all data reported in this study.

Intraocular Pressure Measurement

IOP was measured with a hand-held tonometer (TonoVet) in the awake, restrained rabbit. Topical anesthesia was not necessary for the measurement and no general anesthetic or immobilizing agent was used because the procedure is not painful. Every effort was made to avoid artificial elevation of IOP by avoiding topical anesthesia and by careful and consistent animal handling during each measurement. Each rabbit was acclimatized to the IOP measurement procedure for at least 7 days to obtain a stable background IOP reading. To account for the specific IOP behavior of each rabbit, the initial IOP value (time=0) reported for each individual eye is an average of measurement over 3-4 days and the IOP over time are reported as changes in IOP relative to that initial average value.

Calculation of Area Under the Curve and Equivalent Dosage

The pharmacodynamic effect of each treatment was characterized by determining the area under the curve of the temporal profile of intraocular pressure by numerically integrated using the trapezoidal rule. This pharmacodynamic area under the curve (AUC_{PD}) is a measure of the strength and duration of the treatment on IOP. To make the AUC_{PD} calculation, IOP readings were normalized to the IOP reading prior to the treatment. The obtained value of AUC_{PD} had units of mm Hg-hr and a negative value (because the drugs under study all lowered IOP). However, the negative values were changed to positive values for better representation of the data.

$$A U {C}_{PD} = {\sum }_{i = 1}^{9} [ - ( {t}_{i} - {t}_{i - 1} ) [ \frac{I O P ( {t}_{i - 1} ) + I O P ( {t}_{i} )}{2} ] ] \qquad ( 1 )$$

where IOP(tᵢ) in mm Hg represents the IOP value measured at time tᵢ in seconds.

An equivalent dosage comparison between topical and supraciliary delivery was made using the following equation, where D is the dose administered and the subscripts SC and topical mean suprachoroidal injection and topical administration, respectively.

$$Equivalent dosage = [ \frac{A U {C}_{PD , SC} / {D}_{PD , SC}}{A U {C}_{PD , topical} / {D}_{PD , topical}} ] \qquad ( 2 )$$

Statistical Analysis

Three replicate pharmacodynamics and safety experiments were done for each treatment group, from which the mean and standard error of mean were calculated. Experimental data were analyzed using two-way analysis of variance (ANOVA) to examine the difference between treatments. In all cases, a value of p<0.05 was considered statistically significant. Parametric statistics were used to evaluate the data, as justified by an Anderson-Darling normality test, which showed a normal distribution of IOP measurements in untreated eyes (N=3, p-value=0.367).

Effect of Anesthesia on Transient IOP Change

Before studying the effect of supraciliary targeting of anti-glaucoma drugs, a general anesthetic was identified that does not create artifactual changes in rabbit IOP over the time scale of the experiment. Subcutaneous injections of ketamine/xylazine were tested, which produced deep anesthesia for approximately 2 hours. This anesthetic also produced significant ocular hypotension that lasted for 4-5 hours, with a peak IOP decrease of approximately 5 mmHg at 1 hour after injection of the anesthetic, which was followed by a slow recovery of IOP over time (data not shown).

Isoflurane was then tested, which was administered by inhalation of an escalating dose over 15 min. Anesthesia quickly set in upon initiation of the isoflurane dose and quickly reversed upon discontinuation of the isoflurane dose. During the 15 min of isoflurane administration, IOP was elevated by almost 5 mmHg, but quickly returned to normal after isoflurane administration was stopped, and remained unchanged for 9 hours after that (data not shown). The initial, transient ocular hypertension may have been due to both the pharmacological effect of the anesthetic, as well as the psychological effect (i.e., startling the rabbit) of administering the inhaled anesthetic.

Thus, it was determined that isoflurane was a suitable anesthetic for the pharmacodynamic experiments in this study, because isoflurane's effects on IOP reversed within 15-30 min, which was fast enough to permit hourly measurements of IOP without significant artifact from the anesthetic. However, for the safety experiments in this study in which IOP was measured multiple times within 1 hour, the rapidly changing effects of isoflurane on IOP would significantly affect IOP measurements. For that reason, ketamine/xylazine was used for the safety study, because the effect of the anesthetic on IOP was relatively small during the first 10 min when the most critical IOP measurements were made in the safety study.

Anti-Glaucoma Drugs in the Normotensive Rabbit Model

Anti-glaucoma drugs that have pharmacological action at the ciliary body and reduce IOP in the normotensive rabbit model were identified. Candidates included prostaglandin analogues, adrenergic agonists and beta-blockers that have their pharmacological site of action at the ciliary body. Prostaglandin analogues were preferred because they are widely used in human clinical medicine, including for glaucoma treatment. Latanoprost, travoprost, and bimatoprost are commonly used prostaglandin analogues, but rabbits respond poorly to these drugs. For example, latanoprost was tested in the rabbit model, but no change in IOP was observed at the standard human dose of 2.5 μg (data not shown).

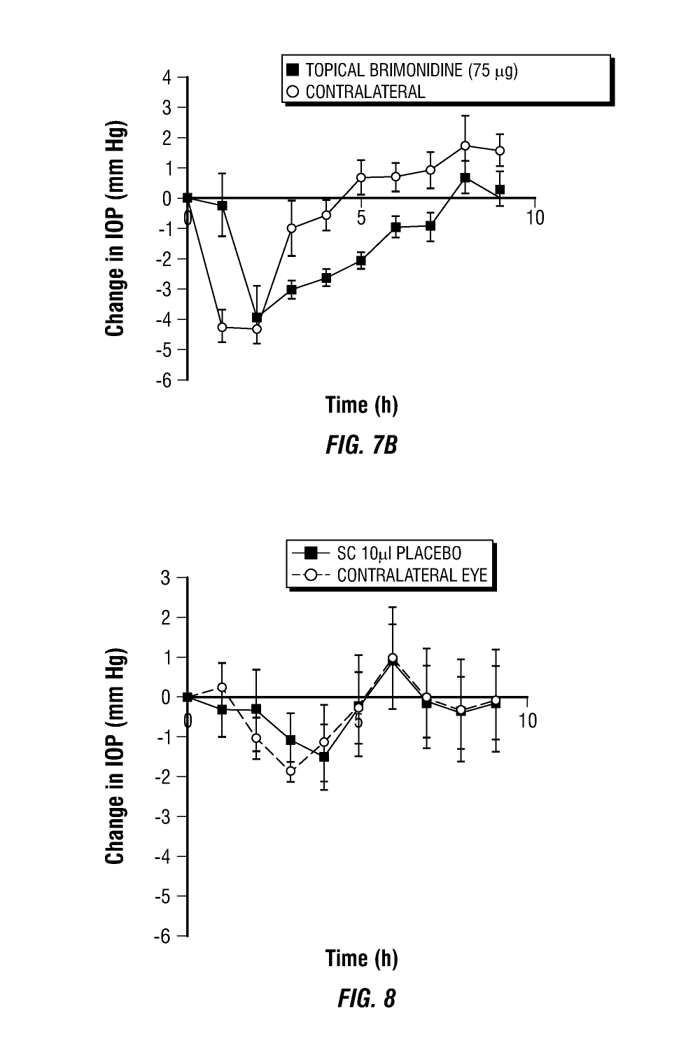

Thus, sulprostone was used as a model prostaglandin analogue with its site of pharmacological action to the ciliary body and an ocular hypotensive effect well documented in literature. A single topical eye drop of 2.5 μg of sulprostone gave a maximum IOP decrease of almost 3.4 mmHg at approximately 2 hours after drug administration (FIG. 7A). Ocular hypotension in the treated eye lasted about 8 hours. Changes in IOP also were observed in the contralateral (i.e., untreated) eye, but to a lesser extent.

A second drug that lowers IOP by a different mechanism in the ciliary body, brimonidine, an adrenergic agonist that is widely used in clinical glaucoma therapy was also evaluated. While the pharmacology and site of action causing an IOP response to brimonidine is species dependent, adrenergic agonists have a site of action in the ciliary body in both the rabbit and human. Topical administration of a single drop (75 μg) of brimonidine produced a peak IOP reduction of approximately 4 mmHg at 2 hours after drug administration, which slowly returned to normal within 6 hours (FIG. 7B). It is notable that the contralateral (untreated) eye also experienced a decrease in IOP with faster kinetics and similar magnitude, presumably due to systemic distribution of brimonidine. The slower kinetics in the treated eye could be explained by a local brimonidine concentration that was initially too high and only after some clearance of the drug reached the optimal concentration for IOP reduction, whereas the contralateral eye had lower brimonidine concentration from the start due to the non-targeted systemic delivery route. Previous research also showed decreased IOP in the contralateral eye in rabbits, which was produced due to systemic administration after administering brimonidine at high concentrations in the treated eye and was reflected by plasma concentrations high enough to activate central α₂-adrenoceptors and cardiovascular changes.

Microneedles for Targeted Delivery to the Supraciliary Space

Targeted injection into the supraciliary space using a microneedle was demonstrated using microneedles measuring 700-800 μm to be inserted to the base of the sclera. The needles were longer than the thickness of the sclera to account for the overlying conjunctiva and for the expected deformation of the sclera during insertion of the microneedle. Previous studies making injections in this way have targeted the suprachoroidal space with the objective of having the injected formulation flow away from the site of injection and travel circumferentially around the eye for broad coverage of the choroidal surface, especially toward the posterior pole. This study had the opposite objective—to localize the injected formulation at the site of injection immediately above the ciliary body and minimize flow to other parts of the eye.

To accomplish this goal, the viscosity of the injected formulation was increased by adding 2% w/v CMC. The viscosity of this solution at rabbit body temperature of 39° C. was 80.5±3.7 Pa-s at a shear rate of 0.1 s⁻¹, which is approximately 80,000 times more viscous than water at room temperature. Injection of this high-viscosity formulation into the rabbit eye using a microneedle was able to localize the injection near the site of injection (data not shown). The dye injected in this way spread over an area within just a few millimeters from the site of injection. The degree of this spread depended on the amount of fluid injected, such that there was more spread when larger volumes were used (data not show).

Histological examination demonstrated that the injection was localized to the supraciliary space. The injected dye was seen in the expanded supraciliary space bounded by the ciliary body on the lower anterior boundary, the choroid on the lower central and posterior boundary and the sclera on the upper boundary of the rabbit eye. A similar experiment was conducted in a human eye, and similarly showed supraciliary localization of the injected fluorescent particles. While the supraciliary space is significantly expanded immediately after injection when these tissues were frozen for analysis, it is believed that this space closes down again as fluid flows away and is absorbed (based on unpublished data on suprachoroidal injections and other data discussed further below).

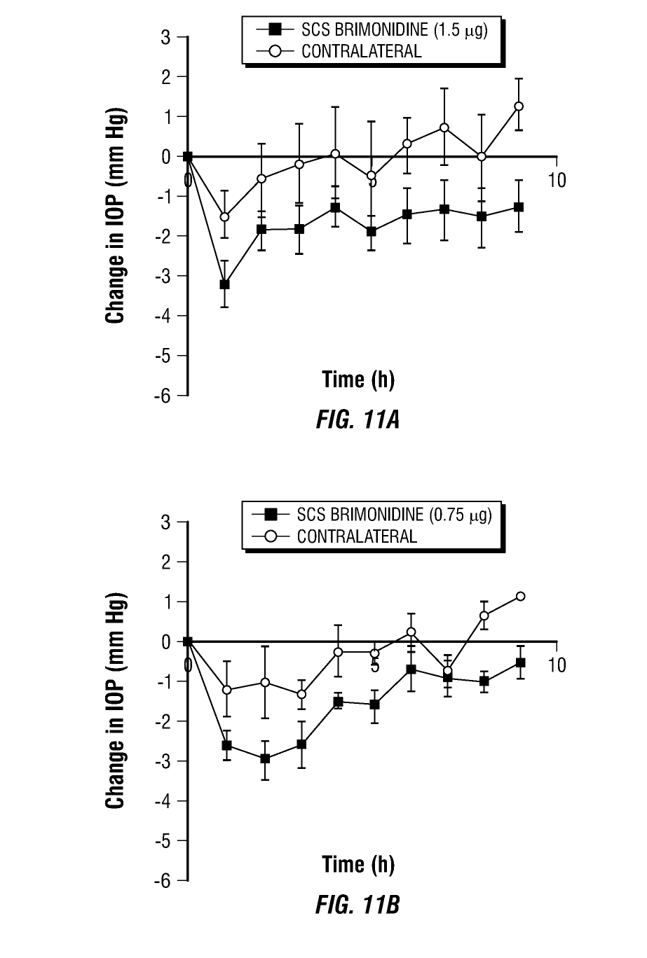

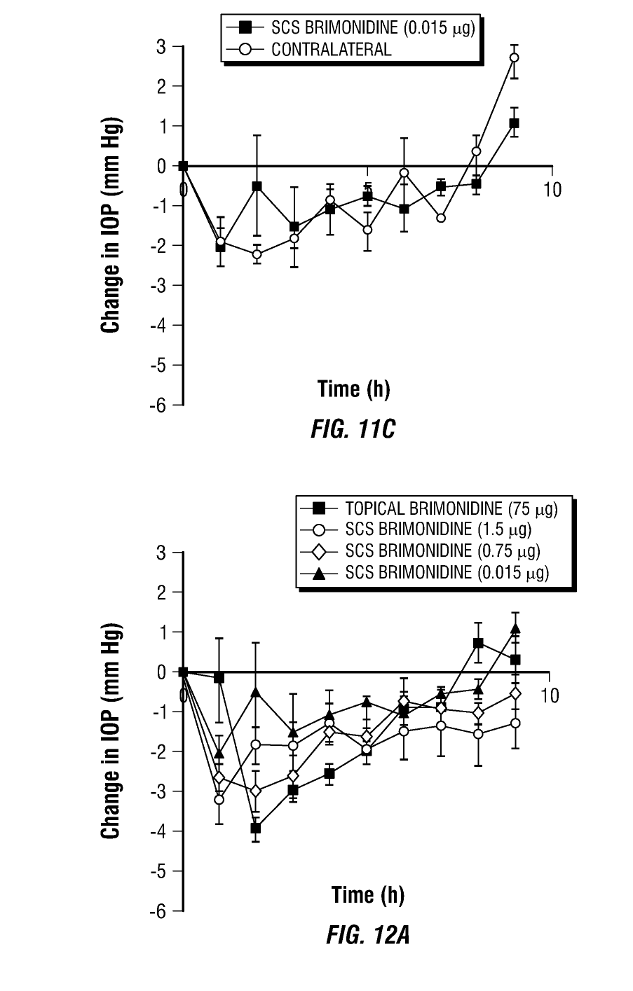

The possible effects of supraciliary injection of 2% CMC in 10 μL on IOP were evaluated over the course of the experiments. As shown in FIG. 8, there was no apparent effect of this injection on IOP at the hourly timepoints over the course of a 9 hour study. A two-way ANOVA comparing the isoflurane-only group (data not shown) to the data in FIGS. 11A-11C showed no statistically significant difference with p-values of 0.05 and 0.07 for treated and contralateral eyes, respectively.

Pharmacodynamics of Sulprostone after Supraciliary Delivery

Having completed the initial experiments on anesthesia, topical delivery and supraciliary targeting, the effects of anti-glaucoma drugs targeted to the supraciliary space were evaluated by injecting sulprostone into the supraciliary space over a range of doses (0.025 μg-0.005 μg in 10 μL) in rabbits.

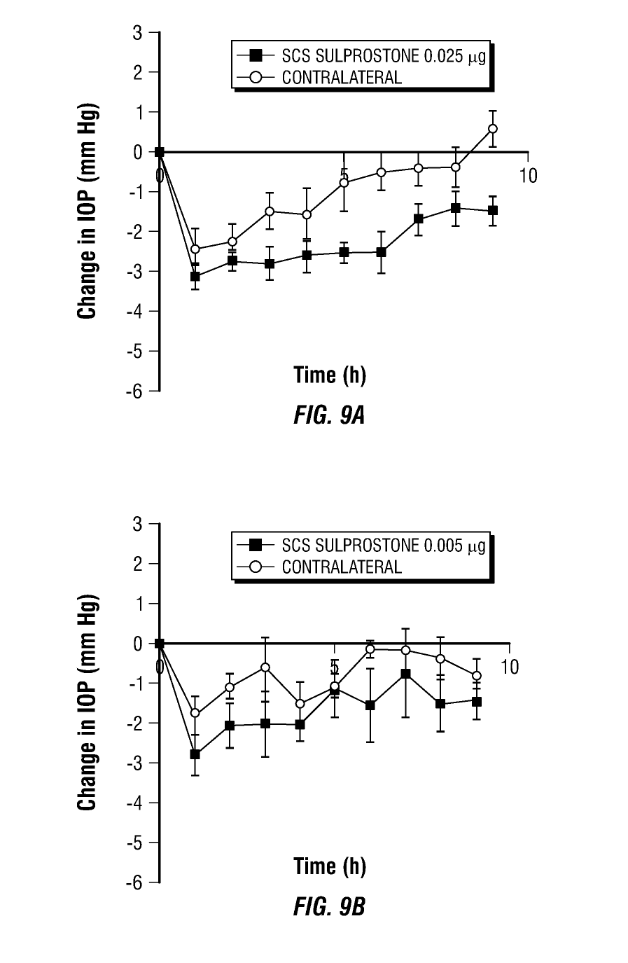

Supraciliary delivery of sulprostone at a dose of 0.025 μg in 10 μL (i.e., a dose 100 times lower than a typical topical dose) produced an IOP decrease of ˜3.1 mmHg within 1 hour that persisted at that level for at least 9 hours (FIG. 9A). IOP was similarly decreased in the contralateral eyes, but to a lesser extent.

Supraciliary delivery of 0.005 μg sulprostone in 10 μL (i.e., a dose 500 times lower than the topical dose) produced a peak IOP drop of ˜2.8 mmHg at 1 hour after drug administration (FIG. 9B). IOP increased over time, but ocular hypotension persisted for the approximately 6 hours in the treated eye and were statistically significant compared to placebo treated eyes (p<0.0001). However, responses of the contralateral eyes were not significantly different from placebo treated eyes (p=0.159).

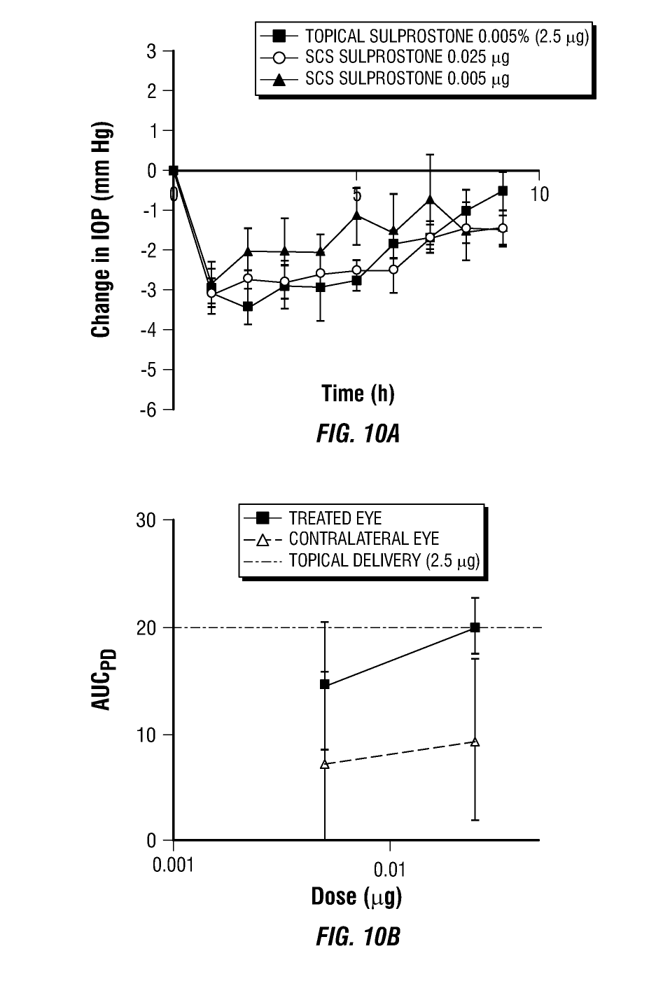

Overall, sulprostone was found to lower IOP in a dose-dependent manner (FIG. 10A). Based on a rough comparison, topical delivery of 2.5 μg sulprostone and supraciliary delivery of 0.025 μg sulprostone in 10 μL showed similar levels of initial IOP reduction, although the effect lasted longer after supraciliary delivery. To provide a more quantitative measure of the supraciliary dose equivalent to topical delivery, the AUC_{PD} for the pharmacodynamic data in the topical and supraciliary treated eyes was determined and compared (FIG. 10B). Comparison of these values gave a ratio of 101, which indicates that the supraciliary dose needed to achieve a similar pharmacodynamic response was ˜100 fold less than for topical delivery. This dramatic dose sparing may have been achieved by highly targeted delivery of sulprostone to its site of action in the ciliary body.

Pharmacodynamics of Brimonidine after Supraciliary Delivery

To assess the generality of dose sparing by targeting anti-glaucoma drugs to the supraciliary space, similar experiments were carried out to study supraciliary delivery of brimonidine over a range of concentrations (0.015 μg-0.15 μg in 10 μL) in rabbits. Similar to sulprostone, brimonidine produced a concentration-dependent drop in IOP at doses much lower than used for topical delivery.

Supraciliary delivery of brimonidine at a dose of 1.5 μg in 10 μL (i.e., a dose 50 times lower than the typical topical dose) produced an IOP decrease of ˜3.3 mmHg within 1 hour that persisted at that level for about 9 hours (FIG. 11A). IOP was similarly decreased in the contralateral eye, but to a lesser extent.

Supraciliary delivery of 0.75 μg brimonidine in 10 μL (i.e., a dose 100 times lower than the topical dose) produced a peak IOP drop of ˜3 mmHg at 2 hours after drug administration that persisted at that level for about 5 hours (FIG. 11B). The contralaterial eye showed a similar, but smaller drop in IOP. Statistical analysis showed significant difference for treated (p<0.001) eyes but not for the contralateral eyes (p=0.915)

Supraciliary delivery of 0.015 μg brimonidine in 10 μL (i.e., doses 500 times lower than the topical dose) showed no significant IOP changes in treated (p=0.20) and contralateral eyes (p=0.26) (FIG. 11C).

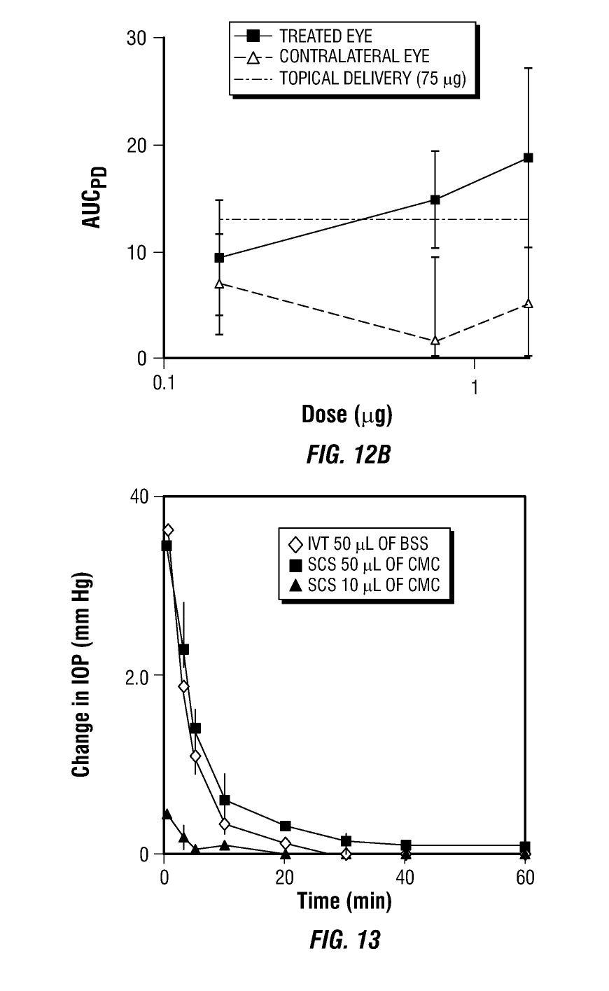

Supraciliary delivery of brimonidine reduced IOP in a dose-dependent matter (FIG. 12A). Compared to topical delivery of 75 μg of brimonidine, a 100-fold lower dose of 0.75 μg of brimonidine by supraciliary delivery showed a similar duration and magnitude of ocular hypotension. By calculating AUC_{PD} values (FIG. 12B), the supraciliary dose needed to get a similar pharmacodynamic response was estimated to be 115-fold less than topical delivery.

It is notable that in the rabbit model studied here, decreased IOP was seen both in the treated eyes and to a lesser extent in the contralateral eyes. Ocular hypotension in contralateral eyes is believed to be due to systemic absorption. Similar contralateral responses were also observed after topical delivery of brimonidine.

Safety of Microneedle Injection into the Supraciliary Space

Injections into the supraciliary space using microneedles were well tolerated and no injection-related complications were observed, such as bleeding or squinting. After injection, the needle insertion site was not visually apparent on the conjunctival surface, indicating only very minor trauma (data not shown). No inflammation, redness, or pain-related response after the injection was observed. No apparent vision loss was observed in any of the rabbits.

To further assess safety, IOP elevation associated with supraciliary and intravitreal injection was measured. Note that this is the short-lived elevation in IOP caused by the injection itself (as opposed to the longer-term IOP reduction caused by the anti-glaucoma drugs presented above). For this study, ketamine/xylazine was used for general anesthesia because it provides a relatively steady IOP between 1 hour and 2 hours after injection. Rabbits given an intravitreal injection of 50 μL of HBSS 1 hour after induction of anesthesia were found to have a peak IOP increase 36±1 mmHg due to the injection (FIG. 13). IOP then decreased exponentially until it stabilized after 30-40 min after the injection. This is similar to what is seen in human patients, where intravitreal injection can increase IOP by ˜30 mmHg. Considering intravitreal injection is well tolerated in human patients using just topical anesthesia and is safely performed millions of times per year, this temporary increase in IOP would be expected to be safe and well tolerated.

A transient increase in IOP that peaked at 35±3 mmHg and decayed in under 1 hour was observed upon injection of 50 μL of a 2% CMC formulation into the supraciliary space of the rabbit eye, which is similar to the effects of conventional intravitreal injection (FIG. 13). The peak IOP increase was 5±1 mmHg upon injection of 10 μL of formulation into the supraciliary space, which then disappeared within 20 min. Considering the similar magnitude and kinetics of IOP change by these intravitreal and supraciliary injection, the safety profile of supraciliary delivery may be similar to that of intravitreal injection. In fact, supraciliary injection may be safer than intravitreal injection, considering that intravitreal and supraciliary injections are performed at the same site of the eye (i.e., pars plana), but supraciliary injection uses a needle that penetrate an order of magnitude less deeply into the eye.

This study introduced the idea of targeting the ciliary body by injection into the adjacent supraciliary space. This space located just a few hundred microns below the conjunctival surface was accessed by using a hollow microneedle designed to be just long enough to penetrate to the base of the sclera. Injection at this site filled the supraciliary space with a formulation designed with high viscosity that inhibited its flow away from the site of injection, thereby creating a depot next to the ciliary body. When anti-glaucoma drugs were injected in this way, they were able to reduce IOP at doses two orders of magnitude lower than those required for similar pharmacodynamics using topical eye drops. These results show the highly targeted nature of supraciliary delivery and suggest opportunities to improve glaucoma therapies.

Moreover, targeted delivery may reduce the amount of drug administered. This can improve safety and patient acceptance, due to reduced side effects. Targeted delivery also facilitates development of sustained-release therapies that eliminate the need for patients to comply with daily eye-drop regimens. For example, brimonidine is used clinically at a daily topical dose of 75 μg given 3 times per day. The daily dose of brimonidine administered to the supraciliary space appears to be approximately 100 times less than the topical dose. This means that the supraciliary daily dose is roughly to be 2.25 μg and a three-month supply would be 67.5 μg. While these calculations suggest the feasibility of injecting controlled-release microparticles into the supraciliary space, additional pharmacokinetics study will be needed to develop such controlled-release microparticles.

If this vision for sustained-release drug therapy can be realized, it could have a dramatic effect on patient compliance with glaucoma therapy. Current therapy requires many patients to administer eye drops on at least a daily basis. Compliance with such dosing schedules is very low, in the range of 56%. Many glaucoma patients visit their ophthalmologists every six months for routine exams. In this way, glaucoma patients could receive supraciliary injections of sustained-release medication during their regular doctor's visits and thereby eliminate the need for compliance with topical eye drop therapy.

From a practical standpoint, supraciliary injections could be relatively easily introduced into clinical practice. Currently, retina specialists give millions of intravitreal injections per year at the pars plana located 2-5 mm from the limbus. Supraciliary targeting requires placement of microneedles at the same site, which should be straightforward for an ophthalmologist to do. Assuring microneedles go to the right depth at the base of the sclera is determined by microneedle length, which is designed to match approximate scleral thickness. Variation of the scleral thickness could be compensated for by the pliable nature of the choroid.

### EXAMPLE 3

Previous studies have used microneedles to inject drug formulations into the suprachoroidal space in a minimally invasive manner. These microneedles are 30- to 33-gauge hypodermic needles that have been laser-machined to a length of less than 1 mm, which allows them to cross the sclera and overlying conjunctiva for precise placement of the needle tip at the suprachoroidal space. This injection procedure, which requires minimal training for an experienced researcher or ophthalmologist, has been used extensively in animals and, more recently, in human subjects. Upon fluid injection, the suprachorodal space can expand to incorporate injected materials, including polymeric particle formulations. Injection of unformulated particles in saline distributes the particles over a portion of the suprachorodial space, but does not target delivery to specific regions within suprachoroidal space. To improve on this technique, a new formulation was developed to deliver nanoparticles to specific sites within the suprachoroidal space using emulsion droplets to target the macula near the back of the suprachoroidal space and to target the ciliary body near the front of the suprachoroidal space.

Fabrication of Particle-Stabilized Emulsion Droplets PEDs

Carboxylate-modified, non-biodegradable, 200 nm diameter, fluorescent polystyrene nanoparticles at an initial concentration of 2% by weight (Fluospheres, Invitrogen, Carlsbad, Calif.) were diluted in BSS to obtain 0.6%, 0.4%, and 0.2% solutions. These solutions were then mixed at a 7:3 ratio by volume with perfluorocarbon (perfluorodecalin, Sigma-Aldrich, St. Louis, Mo.) and homogenized (PowerGen 700, Fisher Scientific, Pittsburgh, Pa.) at setting 5 for 20 sec to form PEDs. The aqueous phase was then removed using pipettor and replaced with 1% polyvinyl alcohol (PVA, Sigma-Aldrich) in BSS solution. The solution was then filtered through various sizes (11, 20, 30, 40 μm) of nylon net filters (Millipore, Billerica Mass.) to obtain desired emulsion droplet sizes. Multiple images of the PEDs were taken using a microscope (IX 70, Olympus, Center Valley, Pa.) and the PED size distribution was measured using ImageJ software (US National Institutes of Health, Bethesda, Md.). The concentration of the PEDs was determined by the volume of settled PEDs per volume of aqueous phase (1% PVA). All the particle sizes were prepared using a concentration of 50 μL of PEDs per 1 mL of aqueous solution (1% PVA).

Microneedle Fabrication

Metal microneedles were fabricated from 30-gauge needle cannulas (Becton Dickinson, Franklin Lakes, N.J.). The cannulas were shortened to approximately 600-700 μm in length and the bevel at the orifice was shaped using a laser (Resonetics Maestro, Nashua, N.H.). The microneedles were electropolished using an E399 electropolisher (ESMA, South Holland, Ill.) and cleaned with deionized water.

Ex Vivo Injection Procedure

Whole New Zealand White rabbit eyes (Pel-Freez Biologicals, Rogers, Ark.) with the optic nerve attached were shipped on ice and stored wet at 4° C. for up to 2 days prior to use. Eyes were allowed to come to room temperature, and any fat and conjunctiva were removed to expose the sclera. A catheter was inserted through the optic nerve into the vitreous and connected to a bottle of Hank's Balanced Salt Solution (BSS, Corning Cellgro, Manassas, Va.) raised to a height to generate internal eye pressure of 10 mmHg, which was used to mimic the lowered intraocular pressure in rabbit eyes under general anesthesia. The eye was positioned with cornea facing up or down, as needed to orient relative to gravity. The microneedle was attached to a gas-tight glass syringe containing the formulation to be injected. The microneedle was then inserted perpendicular to the sclera tissue 3 mm posterior from the limbus in the superior temporal quadrant of the eye. A volume of 200 μL was injected within 3 sec and then an additional 30 sec was allowed before removing the microneedle from the eye to prevent excessive reflux.

In Vivo Microneedle Injection

Microneedle injection was done under systemic anesthesia (subcutaneous injection of a mixture of ketamine/xylazine/ace promazine at a dose of 17.5/8.5/0.5 mg/kg). Topical proparacaine (a drop of 0.5% solution) was given 2-3 min before microneedle injection as a local anesthetic. The rabbit was positioned with cornea facing up or down, as needed to orient relative to gravity. The microneedle was attached to a gas-tight glass syringe containing the formulation to be injected. For a suprachoroidal space injection, the eyelids of the rabbit were pushed back and the microneedle was inserted into the sclera 3 mm posterior to the limbus in the superior temporal quadrant of the eye. A volume of 200 μl was injected within 5 sec and an additional 60 sec was allowed before removing the microneedle from the eye to prevent excessive reflux. The animal was maintained in position and under anesthesia for 30 min after the injection to give enough time for the PEDs to completely settle down and all the aqueous formulation to dissipate out of the suprachoroidal space. At this point, if needed, an injection into the other eye was similarly performed. All experiments were carried out using New Zealand white rabbits with approval from the Georgia Tech Institutional Animal Care and Use Committee, and animals were euthanized with an injection of pentobarbital through the ear vein.

Tissue Processing and Measurement of Fluorescent Intensity

After the suprachoroidal injection, eyes were snap frozen in an isopropyl alcohol (2-isopropanol, Sigma Aldrich) bath, which was cooled in dry ice. After the eyes were completely frozen, they were removed and eight radial cuts were made from the posterior pole toward the anterior segment. After making eight cuts around the ocular globe, each “petal” was peeled away outwardly to expose the inside of the eye. This makes eyes into a flat mount-like “flower-petal” configuration visually exposing the inner side and the injected dyes in the eyes. Brightfield and fluorescence images of the inside of the eyes were imaged to visualize the distribution of fluorescent nanoparticles. Brightfield images were taken using a digital camera (Cannon Rebel Tli, Melville, N.Y.) and fluorescence images were taken using a fluorescence microscope (Olympus SZX16, Center Valley, Pa.). Each of the eight petals was then divided into additional four pieces. Approximate distance from the ciliary body to the back of eye ranged from 1.2-1.4 mm. The cuts were made 3, 6, and 9 mm away from the ciliary body, where the suprachoroidal space starts, producing a total of 32 tissue pieces from each eye. Individual pieces were paired into 4 quadrants resulting in 16 vials each containing two pieces of the tissue in BSS solution. Ocular tissues were then homogenized (Fisher Scientific PowerGen) to extract injected non-biodegradable fluorescent nanoparticles (Figure S4 in Supplemental Information). The aqueous part of the mixture was pipetted out into 96 well plates to measure fluorescence signal intensity (Synergy Microplate Reader, Winooski, Vt.).

Particle-Stabilized Emulsion Droplet Fall Time Measurement

A solution containing 5% by volume PEDs was put into a clear glass vial and vigorously shaken before the start of recording the movement of PEDs using a digital camera (Cannon Rebel Tli). A green light bulb (Feit Electric, Pico Rivera, Calif.) was used to excite the fluorescent nanoparticles surrounding the PEDs and a red camera filter (Tiffen red filter, Hauppauge, N.Y.) was mounted on the digital camera to visualize the movement of the PEDs. The height of the solution was measured and the time it took for essentially all the PEDs to fall to the bottom of the vial was measured.

Particle-Stabilized Emulsion Droplet Fall Time Modeling

The time it took for PEDs to fall to the bottom of the vial was modeled using the following equations.

Fₙₑₜ=F_{g}−F_{B}−F_{D} (3)

ρₒVₒxⁿ(t)=ρₒVₒg+ρ_{f}V_{f}g+6πηrx′(t) (4)

where Fₙₑₜ is the net force, F_{g} is gravitational force, F_{B} is buoyancy force, F_{D} is Stokes drag force, ρₒ is density of the PED (i.e., 1.9 g cm⁻³), ρ_{f} is density of a carrier fluid (i.e., water, 1 g cm⁻³), Vₒ is the displacement volume of a PED (i.e. 1440, 8180, or 22,400 μm³), V_{f} is the displacement volume of the carrier fluid (i.e., 1440, 8180, or 22400 μm³), g is gravitation acceleration (i.e., 9.8 m s⁻²), η is the viscosity of the carrier fluid (i.e., 1 cP), r is the radius of a PED (i.e. 14, 25 or 35 μm), and x(t) is height as a function of time.

Ultrasound Measurement

An ultrasound scanner (UBM Plus, Accutome, Malvern, Pa.) was used to monitor the expansion of the suprachoroidal space. The injection was performed at a superior temporal site (between 1 and 2 o'clock) 3 mm back from the limbus and the ultrasound probe was positioned 45 degrees superior to the injection site (at 12 o'clock) 3 mm back from the limbus. Ultrasonic imaging was conducted before and for 10 min after the injection procedure.

Statistical Analysis

A minimum of three replicate experiments was performed for each treatment group, from which the mean and standard deviation were calculated. Experimental data were analyzed using one-way analysis of variance (ANOVA) to examine the difference between treatments. In all cases, a value of p<0.05 was considered statistically significant.

Results

Stabilization of the emulsion droplets was achieved by controlling two properties of the polymeric nanoparticles. First, the hydrophilicity was controlled such that the nanoparticles prefer to be at the emulsion droplet interface and not in either the surrounding water or the perfluorodecalin core. Thus, polystyrene particles were modified with carboxylate groups on the surface, which provided a zeta potential of −47.5±6.07 mV. Second, the largest possible polymer nanoparticles were used, since larger particles generally enable longer controlled release. It was found that nanoparticles up to 200 nm in diameter could be used, but emulsion droplets were unable to be created using larger nanoparticles (data not shown).

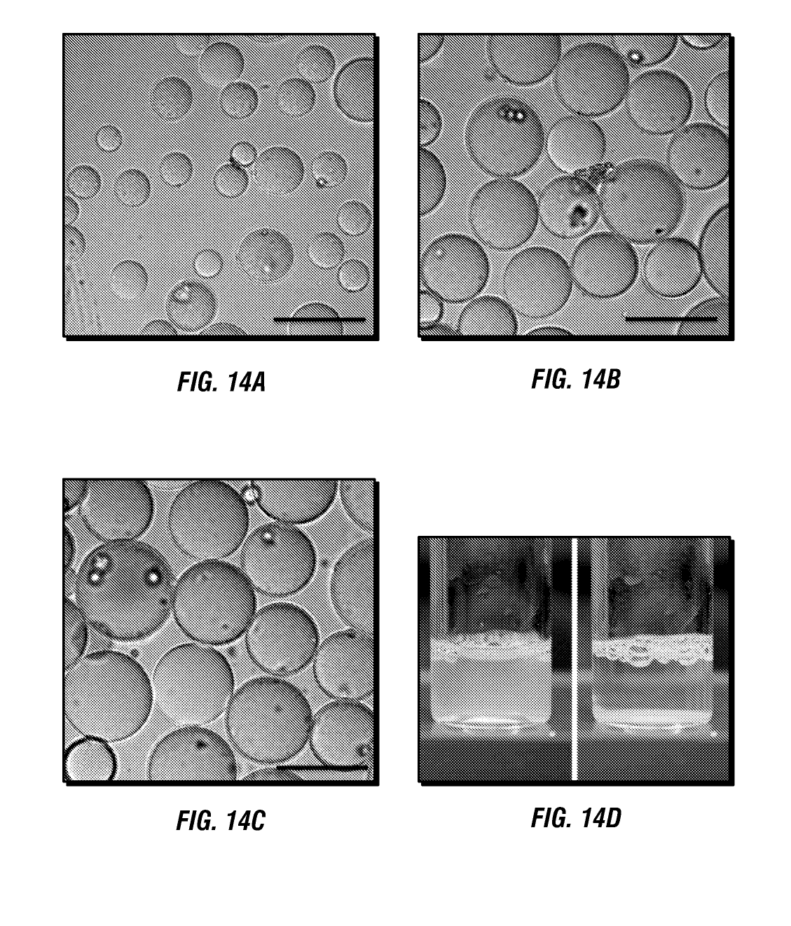

Next, PEDs were made as large as possible to promote rapid settling in the eye due to gravity. PED size was varied by varying the concentration of nanoparticles in the solution when fabricating the PEDs. PED size decreased with increasing nanoparticle concentration (data not shown), which is consistent with observations by others. Increased nanoparticle concentration allows larger surface area coverage of the emulsion droplets, which results in smaller size of PEDs (i.e., higher surface-to-volume ratio). Because PED populations produced in this way were highly poly-disperse, more uniform particle size distributions were prepared by separating the PEDs into size fractions by passing sequentially through nylon net membrane filters of 11, 20, 30 and 40 μm pore size, which produced PED populations of 14±4.3 μm, 25±6.0 μm and 35±7.5 μm diameter (FIGS. 14A-14C). The ability to separate the different PED sizes by filtration showed that the PEDs were mechanically strong enough to withstand the separation process.

As shown in FIG. 14, each PED contained a non-fluorescent interior composed of perfluorodecalin and a film of red-fluorescent nanoparticles around the outer surface. The high-density of the PEDs was demonstrated by rapid settling under gravity, as shown in FIG. 14D. PEDs were designed to fall quickly in the eye due to gravity, with the expectation that larger particles should fall faster than smaller particles due to their increased mass. To determine the fall time of the PEDs in water, which provides an initial estimate of fall time inside the eye after injection, experimental measurements and theoretical calculations were performed. The measured time for PEDS of 14 μm, 25 μm and 35 μm diameter to fall to the bottom of a vial filled with water to a height of 1 cm was 93±3 sec, 54±5 sec, and 31±2.4 sec, respectively (data not shown). A simple force balance to model the process predicted fall times of 104 sec, 32 sec and 16 sec, respectively. The discrepancies between measured and calculated values may be due to variation of the size of and interaction between the PEDs, as well as the subjective nature of experimentally determining when all PEDs settled to the bottom by visualization. In any case, settling times by measurement and calculation were fast, i.e., on the order of 1 min.

Use of Gravity to Target Peds within the Rabbit Eye Ex Vivo

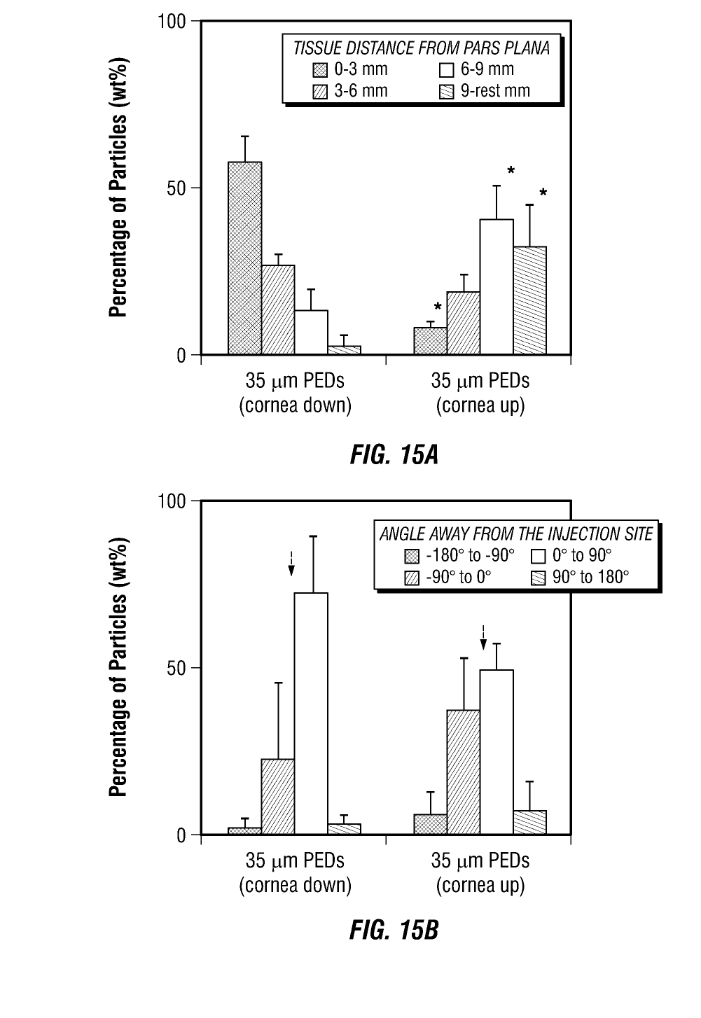

Before conducting in vivo experiments, the hypothesis that deposition of PEDs in eye can be directed by gravity by injecting 35 μm-diameter PED suspensions in the suprachoroidal space of the rabbit eye ex vivo and changing orientation of the eye with respect to gravity was tested. Delivery was first targeted to the anterior portion of the suprachoroidal space by positioning the eye with the cornea facing down and injecting a suspension of PEDs into the suprachoroidal space using a microneedle. The distribution of PEDs after injection was determined by dividing the suprachoroidal space into four antero-posterior quadrants. 59% of the injected PEDs were targeted to the most anterior quadrant, located between the ciliary body and the site of injection 3 mm back from the ciliary body, and 85% were located in the two most anterior quadrants (i.e.,<6 mm from the ciliary body) (FIG. 15A). Particle concentration decreased further back in the eye, with just 2.3% of PEDs in the most posterior quadrant located 9 mm or further back from the ciliary body. There was a statistically significant decrease in PED concentration moving posteriorly within the suprachoroidal space (one-way ANOVA, p=0.0002). This showed significant targeting of the PEDs to the anterior portion of the suprachoroidal space.

Delivery was next targeted to the posterior portion of the suprachoroidal space by positioning the eye with the cornea facing up. In this case, 30% of the injected PEDs were located in the most posterior quadrant adjacent to the macula and 61% were loaded in the two most posterior quadrants (>6 mm from the ciliary body) (FIG. 15A). Just 9.6% were in the most anterior quadrant. There was a statistically significant increase in PED concentration moving posteriorly within the first three quadrants of the suprachoroidal space (one-way ANOVA, p=0.02). This showed significant targeting of the PEDs to the posterior portion of the suprachoroidal space and, when compared with the anteriorly targeted data, demonstrated the gravity-mediated mechanism of the targeting.

Finally, the radial distribution of PEDS to the left and right of the injection site was characterized. As shown in FIG. 15B, the large majority of the particles were located in the upper radial quadrants immediately to the left and right of the injections site (i.e., between −90° to 0° and 0° to 90°) and very little reached the lower radial quadrants (i.e., between −180° to −90° and 90° to 180°). There was no significant difference between the particle concentrations in each of these quadrants as a function of eye orientation (i.e., cornea up versus cornea down, p>0.10). This was expected, because radial movement was in the direction perpendicular to the gravitational field, meaning that gravity should not influence radial movement.

Use of Gravity to Target Peds within the Rabbit Eye In Vivo

Next, injection of 35 μm PEDs into the rabbit eye was repeated in vivo to determine if ex vivo results could be translated to in vivo eyes. The distribution of PEDs in each antero-posterior quadrant of the suprachoroidal space after injection in vivo was not significantly different from injection ex vivo (one-way ANOVA, p>0.7). The radial distributions for in vivo and ex vivo eyes also showed no significant differences (one-way ANOVA, p>0.8). These data showed a good correlation between ex vivo and in vivo injections and demonstrated the use of gravity to target PEDs within the living rabbit eye.

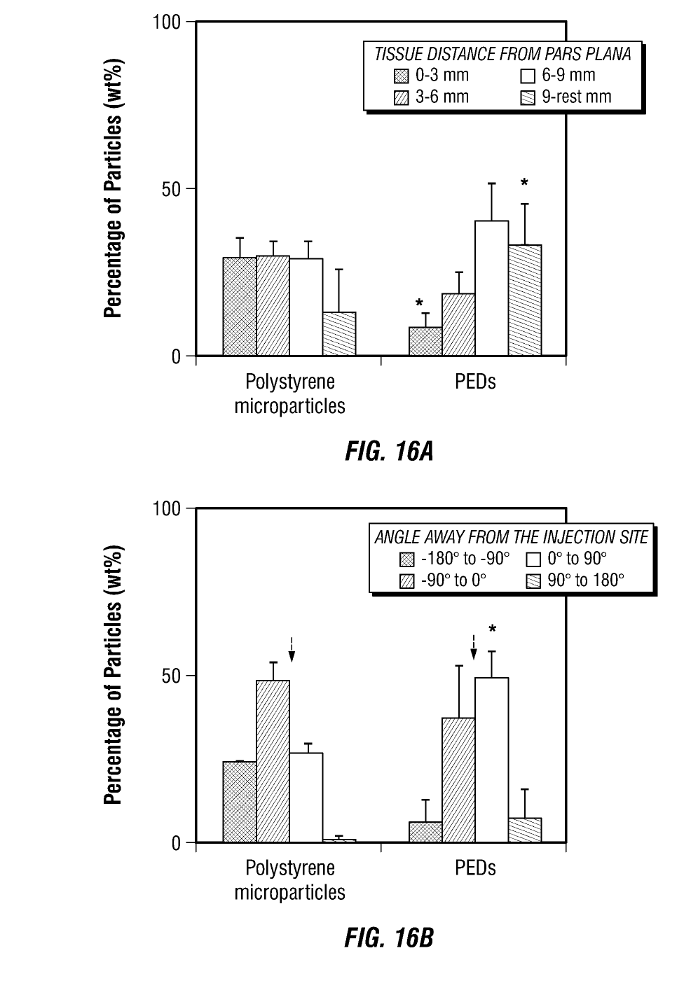

To further assess the role of gravity to target movement of PEDs inside the suprachoroidal space, an identical experiment was carried out ex vivo using fluorescently tagged polystyrene microparticles with a 32 μm diameter that were almost neutral density compared to water (1.05 g cm⁻³) and compared them to PEDs with a 35 μm diameter containing high-density perflurodecalin (1.92 g cm⁻³). The injection conditions in both cases were the same, such as volume injected (200 μL), concentration of particles (5% by volume) and cornea facing up. As shown in FIGS. 16A and 16B, injection of the neutral-density polystyrene fluorescent microparticles resulted in just 13±5% of the particles reaching the most posterior quadrant. In contrast, 2.5 times more of the high-density PEDs reached the most posterior quadrant (i.e., 32±12%). One-way ANOVA analysis showed a statistically significant increase in PED concentration moving posteriorly within the first three quadrants of the suprachoroidal space (one-way ANOVA, p=0.0020). In contrast, there was no statistically significant change in concentration of the polystyrene microparticles within the first three antero-posterior quadrants (one-way ANOVA, p=0.99). The radial distributions showed no significant differences (one-way ANOVA, p>0.10) between PEDs and polystyrene microparticles.

Retention of PEDs at the Site of Targeted Delivery

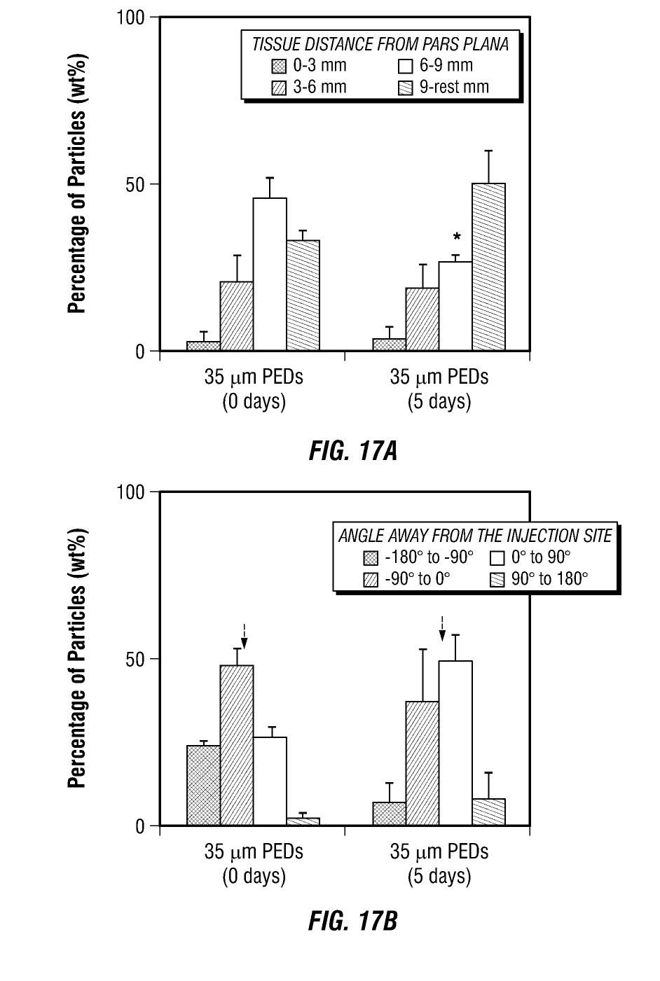

To be most valuable, PEDs should not move around inside the eye after the targeted injection. It was hypothesized that the suprachoroidal space expanded during an injection, but collapsed back to its normal position as fluid dissipated and that this collapse would immobilize the PEDs. To test this hypothesis, PEDs were injected into the left-side eyes of rabbits in vivo with the cornea facing up to localize PEDs to the back of the eye. After five days, during which time the rabbits were allowed to move freely, identical injections were made into the right-side eyes and the animals immediately sacrified to compare PED distribution immediately after and five days after injection. As shown in FIGS. 17A and 17B, the distribution of PEDs in both cases showed a similar trend of increasing PED content toward the back of the eye. After five days, 50% of the injected PEDs were located in the most posterior quadrant adjacent to the macula and 77% were loaded in the two most posterior quadrants (>6 mm from the ciliary body) (FIG. 17A). Statistical analysis (one-way ANOVA) between antero-posterior tissue segments in the two groups were not significant different (p>0.01), except in the 6-9 mm segment (p=0.032). The radial distributions for eyes at 0 days and 5 days after injection showed no significant differences (one-way ANOVA, p>0.25). Thus, it was concluded that PEDs could be targeted to regions of the suprachoroidal space during injection and then could be retained at the site of targeted delivery afterwards. Additional studies will be needed to further assess this retention of PEDs over longer times and, eventually, in humans.

Effect of PED Size on Gravity-Mediated Targeting

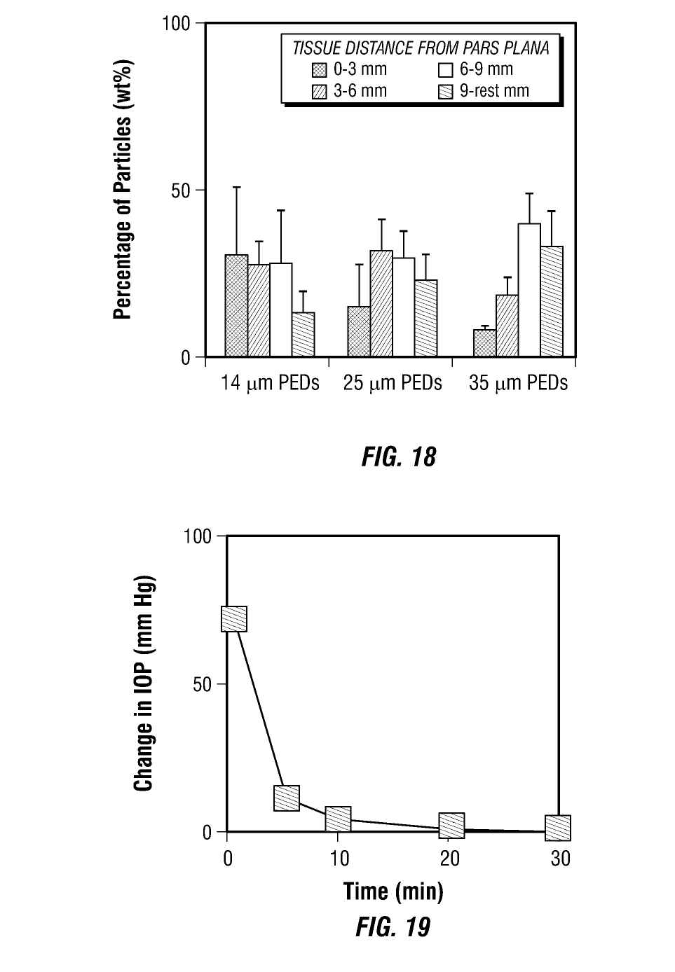

As a further test of gravity-mediated delivery, the mobility of PEDs inside the suprachoroidal space as a function of PED size was measured, with the expectation that larger PEDs should be better targeted by gravity due to their faster fall time. PEDs of 14 μm, 25 μm and 35 μm diameter (see FIG. 14) were injected into the suprachoroidal space and measured the extent of posterior targeting with the cornea facing up in the rabbit eye in vivo. As shown in FIG. 18, PED concentration increased significantly when moving posteriorly within the first three quadrants of the suprachoroidal space for the 35 μm PEDs (one-way ANOVA, p=0.002), but not for the smaller PEDs (one-way ANOVA, p>0.81). This suggested that 35 μm PEDs are optimal for gravity-mediated targeting among the PED sizes tested. It is possible that still-larger PEDS would provide even better targeting by gravity; however, if the PEDs become too large their movement in the suprachoroidal space and in the microneedle during injection may be hindered.

Kinetics of Suprachoroidal Space Collapse

An important parameter that could affect the movement of PEDs in the suprachoroidal space is the time it takes for the suprachoroidal space to collapse after the injection and thereby prevents further movement of the PEDs. Because larger particles were able to more effectively target the back of the eye (FIG. 18) and because these particles have a 1-cm fall time on the order of 1 min (FIG. 14), it was hypothesized that the suprachoroidal space would collapse on a similar timescale on the order of 1 min.

To test this hypothesis, the time it takes for fluid to dissipate from the suprachoroidal space was determined by two methods. First, intraocular pressure (TOP) was measured over time after injection as an indirect measure of suprachoroidal space expansion. As shown in FIG. 19, IOP increased by 72 mmHg upon injection, substantially dropped within 5 min and then returned to baseline IOP within 20 min. The initial increase in IOP is believed to be due to introduction of additional fluid into the eye. This effect is seen after intravitreal injection as well. The decay in IOP is believed to be due to clearance of the fluid from the eye. These data therefore suggest that fluid that is injected into the suprachoroidal space is largely cleared from the eye within 5 min and completely within 20 min. This measurement may provide an overestimate of the time for suprachoroidal space collapse, because fluid in the suprachoroidal space may first redistribute within the eye (which could collapse the suprachoroidal space, but not reduce IOP) and then be cleared from the eye (which would reduce IOP).

The second method used to assess the kinetics of suprachoroidal space collapse employed ultrasound imaging to directly measure the height of the suprachoroidal space over time at a single location. Measurements by ultrasound at a location 45° away radially from the injection site showed immediate expansion of the suprachoroidal space to as much as ˜1000 μm spacing, followed by substantial collapse within tens of seconds. This more direct measurement may provide a more accurate estimate of suprachoroidal space collapse time. This rapid collapse of the suprachoroidal space could explain why 35 μm PEDs showed better movement towards the back of the eye compared to smaller PEDs (FIG. 18).

While most efforts to target drug delivery for ocular applications seek to preferentially deliver drugs to the eye as opposed to other parts of the body, more recent efforts have emphasized more-precisely targeted delivery that directs drug delivery within the eye to specific sites of drug action. Targeting was achieved in this study through the use of high-density PEDs that could be moved by gravity. The design of PEDs achieved gravity-mediated delivery using a perfluorodecalin core stabilized with polymeric nanoparticles that could be adapted in the future for controlled release of encapsulated drugs. While liquid perfluorodecalin was chosen to provide high density and solid polymer nanoparticles to provide future controlled release functionality, alternative designs might choose different materials or combinations of materials to achieve these two capabilities.

For possible future clinical use of PEDs for targeted drug delivery in the eye, it is envisioned that patients will lie down on an exam table (either face up or face down, depending on whether posterior or anterior targeting is needed) for a period of time after receiving an injection to let the PEDs move to their target location while the suprachoroidal space collapses.

### EXAMPLE 4

The location of the suprachoroidal space adjacent to the sites of pharmacological action for diseases like glaucoma (ciliary body) and wet AMD, diabetic retinopathy, and uveitis (choroid and/or retina) may provide a route of administration that enables delivery of higher drug levels in these target tissues. While suprachoroidal space injection enables improved drug targeting, this study sought still better targeting by controlling delivery within the suprachoroidal space. Using conventional formulations, the particles injected into the suprachoroidal space spread over a portion of the suprachoroidal space, but are not well targeted either to localize anteriorly adjacent to pharmacological sites of action in the ciliary body or to spread posteriorly across the whole choroidal surface adjacent to pharmacological sites of action in the choroid and/or retina.

To improve targeting within the suprachoroidal space, formulations were developed either to immobilize particles at the site of injection or to enhance the spreading of the particles throughout the suprachoroidal space. The distribution of particles was determined after injection into the suprachoroidal space as a function of particle size in polymer-free saline formulation. The extent to which polymeric formulation could affect the distribution of microparticles inside the suprachoroidal space was evaluated, with the objective of delivering particles localized immediately above the ciliary body or distributed throughout the suprachoroidal space. To image and quantify movement of particles, non-biodegradable fluorescent particles were used throughout the study. For the first time, this study presents methods to deliver particles up to 10 μm in size targeted to the ciliary body or throughout the choroid using non-Newtonian formulations of polymers having different viscosity, molecular weight and hydrophobicity.

Microneedle Fabrication

Microneedles were fabricated from 33-gauge needle cannulas (TSK Laboratories, Tochigi, Japan). The cannulas were shortened to approximately 750 μm in length and the bevel at the orifice was shaped using a laser (Resonetics Maestro, Nashua, N.H.). The microneedles were electropolished using an E399 electropolisher (ESMA, South Holland, Ill.) and cleaned with deionized water, as described previously. Microneedles were attached to gas-tight, 100-250 mL glass syringes (Thermo Scientific Gas-Tight GC Syringes, Waltham, Mass.) containing the formulation to be injected.

Formulations

Solutions for injection were prepared by mixing 2 wt % FluoSpheres in water (Invitrogen, Grand Island, N.Y.), 0.2 wt % Sky Blue particles in water (Spherotech, Lake Forest, Ill.) and Hank's balanced salt solution (BSS, Manassas, Va.) containing polymer formulations described below at a volumetric ratio of 1:1:2. When carboxymethyl cellulose or methyl cellulose were used, they were dissolved in deionized water rather than BSS. Fluospheres were labeled with red-fluorescent dye and Sky Blue particles were labeled with infrared-fluorescent dye. Particles having diameters of 20 nm, 200 nm, 2 μm or 10 μm were used, but in a given formulation, only one diameter particle was used, and the FluoSpheres and Sky Blue particles both had the same diameter. The polymeric formulations were made using carboxymethyl cellulose (Sigma Aldrich, St. Louis, Mo.), hyaluronic acid (R&D Systems, Minneapolis, Minn.), methylcellulose (Alfa Aesar, Ward Hill, Mass.) or DiscoVisc® (Alcon, Fort Worth, Tex.).

Viscosity Measurements

The viscosity (η) measurements were carried out on an MCR300 controlled-stress rheometer (Anton Paar, Ashland, Va.) equipped with Peltier elements for temperature control and an evaporation blocker that enables measurements of polymer solutions at elevated temperature in a cone-plate geometry. The viscosities of samples were measured at shear rates from 0.01 s⁻¹ to 100 s⁻¹. The viscosity reported for each sample in this study was matched at a shear rate of 0.1 s⁻¹. Multiple measurements were performed, and the mean value is reported.

Ex Vivo Injection Procedure

Whole rabbit eyes were obtained with the optic nerve attached (Pel-Freez Biologicals, Rogers, Ark.). Eyes were shipped on ice and stored wet at 4° C. for up to 2 days prior to use. Before use, eyes were allowed to come to room temperature, and any fat and conjunctiva were removed to expose the sclera. A catheter was inserted through the optic nerve into the vitreous and connected to a bottle of BSS raised to a height that generated an internal eye pressure of 10 mmHg, which mimics the lowered intraocular pressure in the rabbit eye under general anesthesia. The microneedle was then inserted perpendicular to the sclera surface 3 mm posterior from the limbus. A volume of 50 μL or 100 μL was injected within 15 sec, followed by a 30 sec delay before removing the microneedle from the eye to prevent excessive reflux.

In Vivo Injection Procedure

Microneedle injections were carried out in New Zealand White rabbits (Charles River Breeding Laboratories, Wilmington, Mass.). All injections were done under systemic anesthesia by subcutaneous injection of a mixture of ketamine/xylazine/acepromazine at a concentration of 17.5/8.5/0.5 mg/kg. A drop of 0.5% proparacaine was given 2-3 min before injection as a topical anesthetic. To perform a suprachoroidal space injection, the eyelid of the rabbit eye was pushed back and the microneedle was inserted into the sclera 3 mm posterior to the limbus in the superior temporal quadrant of the eye. A volume of 50 μL or 100 μL was injected within 15 sec, followed by a 30 sec delay before removing the microneedle from the eye to prevent excessive reflux. At terminal study endpoints, rabbits were euthanized with an injection of pentobarbital through the ear vein. The eyes were enucleated after death and processed for further analysis. All animal studies were carried out with approval from the Georgia Institute of Technology Institutional Animal Care and Use Committee (IACUC).

Tissue Processing and Measurement of Fluorescence Intensity

Immediately after suprachoroidal space injection into rabbit eyes ex vivo and immediately after enucleation of rabbit eyes in vivo, eyes were snap frozen in an isopropyl alcohol (2-isopropanol, Sigma Aldrich, St. Louis, Mo.) bath, which was cooled in dry ice. After the eyes are completely frozen, they were removed and eight radial cuts were made from the optic nerve on the posterior side to the limbus on the anterior side of each eye. Each of the eight pieces of cut tissue was then peeled away outward exposing the chorioretinal surface inside the eye. This made the eyes into a flat mount-like configuration, exposing the injected dyes for imaging. Brightfield and fluorescence images were taken using a digital camera (Cannon Rebel Tli, Melville, N.Y.). A green light bulb (Feit Electric, Pico Rivera, Calif.) was used to excite the fluorescent particles and a red camera filter (Tiffen red filter, Hauppauge, N.Y.) was mounted on the digital camera to image the distribution of particles inside the suprachoroidal space.

Obtained images were used to quantify the suprachoroidal space area containing injected particles using Adobe Photoshop (Adobe, Jan Jose, Calif.). Each of the eight tissue pieces was then divided into additional two pieces. The cuts were made 6 mm antero-posteriorly from the ciliary body, which is approximately at the mid-point of the suprachoroidal space. In this study, ocular tissue between 0 mm and 6 mm from the ciliary body are referred to as “anterior SCS” and ocular tissue more than 6 mm away from the ciliary body as “posterior SCS”.

This method produced a total of 16 tissue pieces from each eye. Each of the 16 pieces was then put into separate vials containing BSS and homogenized (Fisher Scientific PowerGen, Pittsburgh, Pa.) to extract injected fluorescent particles. The liquid part of the homogenate was pipetted into 96-well plates to measure fluorescent signal intensity (Synergy Microplate Reader, Winooski, Vt.). To quantify radial distribution of particles, data were designated into two categories radially: ocular tissue between −90° and 90° from the injection sites (referred to as “superior SCS”) and ocular tissue between 90° and 270° from the injection site (referred to as “inferior SCS”).

Statistical Analysis

Replicate experiments were done for each treatment group, from which the mean and standard deviation were calculated. Experimental data were analyzed using both one- and two-way analysis of variance (ANOVA) to examine differences between treatments. In all cases, a value of p<0.05 was considered statistically significant.

Distribution of Nanoparticles and Microparticles in the Suprachoroidal Space

Fluorescently tagged, polystyrene particles with various diameters (20 nm, 200 nm, 2 μm, 10 μm) were suspended in 50 μL of HBSS and injected into the suprachoroidal space of New Zealand White rabbit eyes using a hollow microneedle inserted 3 mm posterior to the limbus. The distribution and number of particles in the suprachoroidal space was determined immediately after injection into rabbit cadaver eyes ex vivo and was determined 14 or 112 days after injection into living rabbit eyes in vivo.

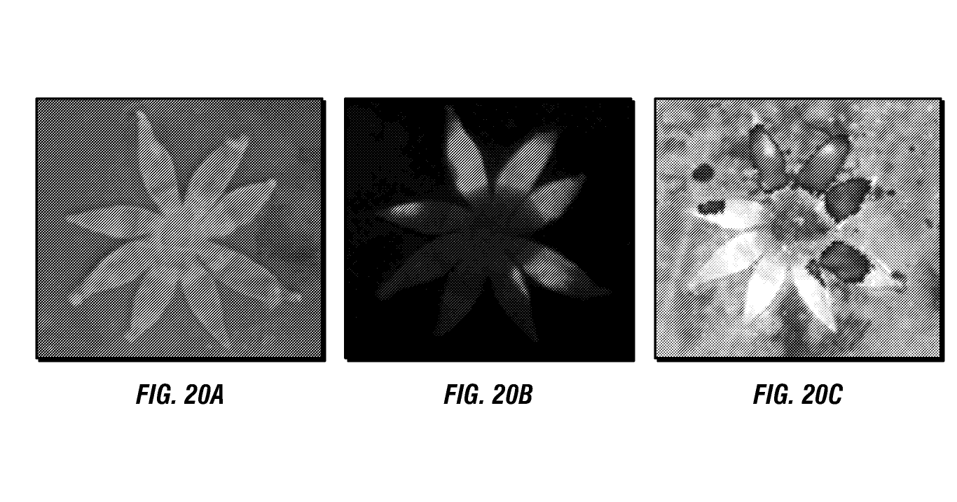

FIG. 20 displays images of a representative eye cut open in a flat-mount presentation showing the distribution of fluorescent particles in the suprachoroidal space. FIG. 20A shows a brightfield image, where the lightly colored interior region is the lens and the tips of the “petals” all were formally joined at the optic nerve before dissection and mounting. FIG. 20B and FIG. 20C show the distribution of red-fluorescent and infrared-fluorescent particles, respectively, which exhibit similar distributions after co-injection. The site of brightest fluorescence intensity corresponds approximately to the site of injection. The sharp circular line where fluorescent signal abruptly ends toward the center of the tissue is interpreted as the anterior end of the suprachoroidal space near the limbus. Quantitative analysis of images like these was used to generate the suprachoroidal space surface area coverage data described immediately below.

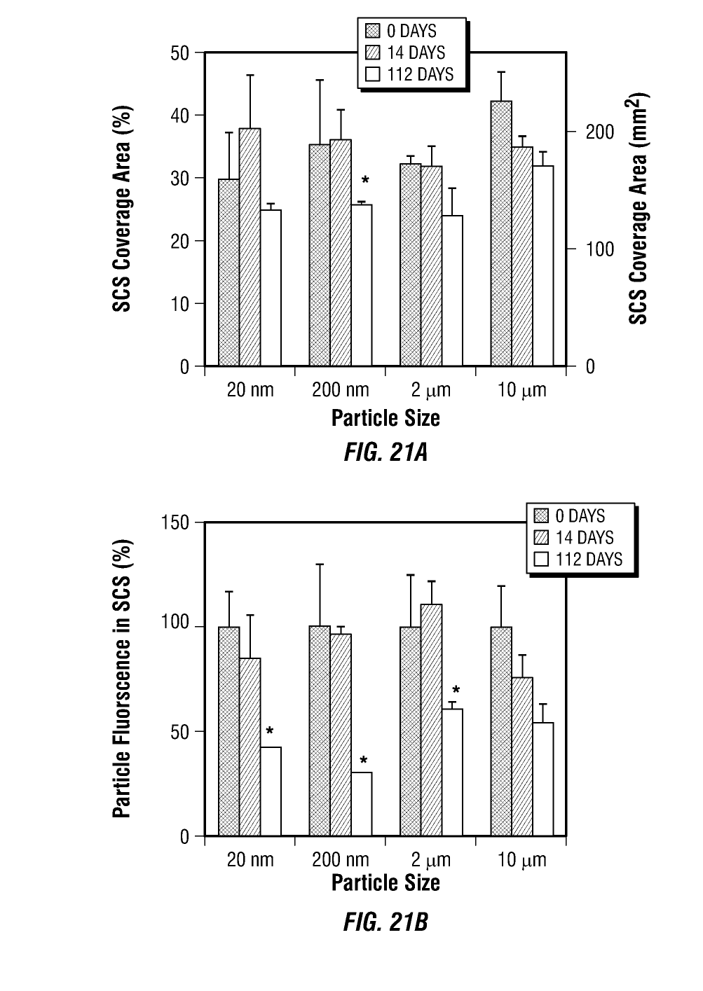

As shown in FIGS. 21A and 21B, immediately after injection, particles covered 29%-42% of the suprachoroidal space surface area. There was no significant effect of particle size on suprachoroidal space surface area coverage (one-way ANOVA, p>0.10). Fourteen days after injection, the suprachoroidal space coverage area did not significantly change for any of the particle sizes studied. Two-way ANOVA analysis showed no significant effect of particle size or time on suprachoroidal spce surface coverage area at 0 and 14 days after injection. Likewise, there was no significant interaction between particle size and time (p=0.16). It is worth noting that the day 0 measurements were made ex vivo, whereas the day 14 measurements were made in vivo, yet the results are similar. Between days 14 and 112, there was a significant decrease in the suprachoroidal space coverage area to 24%-32% of the suprachoroidal space. This represents a reduction of 9%-35% of suprachoroidal space coverage area relative to the day 14 value. Two-way ANOVA analysis showed a significant difference in suprachoroidal space coverage area between day 14 and 112 (p<0.001), but there was no significant effect of particle size (p=0.17). There was also no significant interaction between time and particle size (p=0.21).

In addition to measuring suprachoroidal space coverage area, fluorescence signal intensity of the particles was measured. The fluorescence signal intensity of particles in the SCS between days 0 and 14 showed no significant difference (two-way ANOVA) as a function of time (p=0.13) and particle size (p=0.05). There was also no significant interaction between time and particle size (p=0.1). This suggests that there was no significant clearance of particles during the first 14 days after injection.

However, the fluorescence intensity from particles decreased between days 14 and 112, as shown by fluorescence intensities of 31%-61% of original values (at day 0). This suggests a 39%-69% reduction in the number of particles remaining in the suprachoroidal space. Two-way ANOVA analysis showed a significant difference in particle fluorescence between days 14 and 112 (p<0.001), but not as a function of particle size (p=0.17). There was also no significant interaction between time and particle size (p=0.21).

Loss of fluorescence from particles may either be due to removal of the particles (e.g., by macrophages) or a reduction of the fluorescence signal intensity over time (i.e., artifact). To assess the relative roles of these two possible mechanisms, the decrease in fluorescence intensity of 20 nm, 200 nm, 20 μm, and 10 μm particles in HBSS was measured after storage for 112 days in the dark at 39° C. to mimic conditions in the suprachoroidal space of the rabbit eye. These particles lost 25±6.5% of their fluorescence signal intensity. This suggests that particle clearance from the eye may not be as extensive as reported, because loss of fluorescence signal may at least partially explain the loss.

Overall, these data show that the injected particles spread over a coverage area of about one-third of the suprachoroidal space. Within 14 days, there was little movement or loss of particles in the suprachoroidal space, but after 112 days, there was a reduction in coverage area to about one-quarter of the suprachoroidal space and there was an apparent reduction in the number of particles in the suprachoroidal space of up to about half of the particles originally injected.

Polymer Characterization

The main objective of this study was to develop formulations that target delivery within the suprachoroidal space. For treatment of macular degeneration, uveitis and other chorioretinal diseases, the spread of injected formulations throughout the suprachoroidal space was sought. For treatment of glaucoma, the ciliary body was targeted by immobilizing injected formulations at the injection site. In particular, it was desired to provide delivery of polymeric particles that simulate controlled-release formulations and to use materials expected to be safe based on prior use in parenteral formulations.

When designing formulations to achieve this objective, two time scales for particle transport were considered. One was during the injection itself and the other was after the injection is over. The data indicated that a simple HBSS formulation enabled spread at the time of injection over about one-third of the suprachoroidal space and that no significant further spreading occurred afterwards. This amount of spreading was too little for complete suprachoroidal space coverage and too much for localized delivery at the site of injection.

Prior studies indicated that the suprachoroidal space closes within minutes after saline injection, which then appears to trap particles in place, which is consistent with the data obtained in this study. Thus, it was hypothesized that addition of polymer to the injected formulation could slow down clearance of the formulation from the suprachoroidal space, thereby allowing it to keep the suprachoroidal space open for longer due to smaller polymer diffusivity and increased solution viscosity. This would allow particles to distribute further within the suprachoroidal space after injection through the expanded suprachoroidal space. Because it is desired to inject as easily as possible (i.e., low injection pressure) and distribute the particles as much as possible during the injection (i.e., throughout the suprachoroidal space), low viscosity at high shear is desired during injection. The shear rate during injection through the microneedles was estimated, but because slow clearance of the polymer was desired after injection, a high polymer molecular weight and concentration and a high solution viscosity at low shear were desired after injection. Thus, it was expected the shear rate after injection should be close to zero.

Hyaluronic acid (HA) was selected as a material that meets these criteria. HA is extensively used in the eye with an excellent safety record. It also exhibits shear-thinning non-Newtonian behavior, so that it has low viscosity during injection and high viscosity afterwards. It is also available at high molecular weight (i.e., 950 kDa). In addition to a pure HA solution, the use of a commercial product, DisCoVisc (DCV), which is a dispersive and cohesive viscoelastic material used in ophthalmic surgery, was studied. DCV contains 17% (w/v) HA (1.7 MDa), as well as sodium chondroitin sulfate (22 kDa). Both a pure HA formulation and the DCV formulation exhibited similar rheological behavior. At high shear rate, the viscosity was low, but at low shear rate it was almost two orders of magnitude higher.

For immobilizing particles, a formulation that gels was needed to hold the particles in place. But a formulation also was needed that has significant viscosity initially to localize the injected formulation during the injection procedure. Thus, it was desired that the formulation resist initial spreading of polymeric particles after the injection and deliver polymeric particles for a long-term sustained release. For targeting the ciliary body, injected particles should be immobilized at the site of the injection and immediately above the ciliary body. Many in situ gelling polymers such as solvent removal, temperature, pH, or light mediated did not have the necessary characteristics. Thus, instead of using existing methods, shear rate mediated systems were selected. There is large difference in shear stress during the injection procedure. While fluid is flowing through the needle, the fluid experiences large shear stress. However, upon injection into the tissue, the fluid experiences extremely low or no shear stress. Therefore, it was hypothesized that strongly non-Newtonian material resists spreading of embedded particles away from the injection site due to its high viscosity at low shear rate.

Polysaccharides were examined as potential formulation to immobilize particles inside the suprachoroidal space due to its excellent biocompatibility. 700kDa carboxymethylcellulose (CMC) and 90 kDa methylcellulose (MC) were selected as potential materials to immobilize polymeric particles due to many of its favorable characteristics. Both 700 kDa CMC and 90 kDa MC are shear-thinning materials that have low viscosity at high shear stress, but that restores its high viscosity at low shear rate. Rheological analysis showed these materials are extremely strongly non-Newtonian. After injection, the materials' high viscosity immobilized the injected particles in the suprachoroidal space. The shear-thinning properties of the CMC come from the high molecular weight nature of the material. Rheological analysis of lower molecular weight (90 and 250 kDa) CMC showed this property. In addition, this shear thinning property lowers the pressure required to achieve successful injection of a high viscosity material during the injection procedure.

To test the hypothesis that high molecular weight and weakly non-Newtonian polymers enhance the spreading of polymeric particles inside suprachoroidal space, both pure HA and DisCoVisc® (DCV, a viscoelastic surgical material) were evaluated. The main component in DCV is HA and shows similar rheological characteristics. In addition to the DCV formulation, 2× and 4× the concentration of DCV were evaluated to study the effect of concentration. The hypothesis was that an increase in concentration would enhance spreading due to the increased time for the suprachoroidal space to stay open for particles to mobilize inside the suprachoroidal space. To quantify the spreading of particles inside the suprachroroidal space, the suprachoroidal space coverage area immediately after the injection was compared to that 14 days after injection. All the initial suprachoroidal space coverage area was done in ex vivo eyes (Pel-Freez Biologicals). The suprachoroidal space coverage area of the BSS formulation was also done as a comparison Immediately after the injection, the particles with polymeric formulations covered 8.3%-11% of the suprachoroidal space surface area. This was expected because the formulation was viscous. In contrast, the BSS formulation covered 42% of the suprachoroidal space initially.

Fourteen days after injection, the suprachoroidal space coverage area drastically changed for all the HA based formulations. Suprachoroidal space surface coverage areas for 950 kDa HA, 1×, 2×, 4×-DCV formulation covered 61%-85% of the suprachoroidal space surface. This represented a 5.7- to 8.7-fold increase in suprachoroidal space coverage area for HA based formulation between days 0 and 14. These significant changes in suprachoroidal space coverage area showed HA based formulations are capable of enhancing spreading of embedded particles. In comparison to BSS formulation, the polymeric formulations showed a 0.77-1.3 fold increase in suprachoroidal space coverage areas after 14 days. One-way ANOVA analysis of BSS and polymeric formulations (950 kDa HA, 1×, 2×, 4×-DCV) showed p-values of 0.018, 0.00052, 0.0094, and 0.0019, respectively. Statistically significant difference was shown for all the HA based formulations. The results also showed the higher concentration of HA formulation resulted in an increase in suprachoroidal space coverage area of the delivered particles. Although there was an increase in coverage area between 1× and 2×-DCV formulation, no statistically significant increase in coverage area was observed between 2× and 4×-DCV formulations.

In an effort to examine if a polymeric formulation could be used to cover the entire suprachoroidal space, an increased volume (100 μL) of 4×-DCV formulation was tested. The results showed the coverage of the entire suprachoroidal space coverage area with a single injection after 14 days. This is a 2-fold increase in the coverage area compared to 100 μL in BSS formulation. One-way ANOVA analysis of BSS (100 μL) and polymeric formulations (4×-DCV-100 μL) showed a p-value of less than 0.0001. This represented a 4.6 fold increase in suprachoroidal space coverage area for 4×-DCV-100 μL between days 0 and 14.

Physical delivery of particles to the targeting site is important, but how much can be delivered is also an important factor to consider. In addition to suprachoroidal space coverage area, particle weight percent distribution was measured antero-posteriorly to characterize the mobility of particles inside the suprachoroidal space. For the in vivo experiment, the portion of particles (%) in the posterior suprachoroidal space for 950 kDa HA, DCV (50 μL), 2× DCV (50 μL), 4× DCV (50 μL), and 4× DCV (100 μL) formulation were 31-49%. Likewise, the portion of particles (%) for 50 μL and 100 μL BSS formulation was 29±15% and 48±2.9%. One-way ANOVA analysis (equal volume) of BSS and HA formulations showed p-values of 0.69, 0.021, 0.0070, 0.012, and 0.017, respectively. Statistically significant difference was found for all the DV formulations.

The portion of particles (%) radially in the superior and inferior suprachoroidal space also was measured. The portion of particles in the inferior suprachoroidal space was 22-30% of injected particles. Likewise, 50 μL and 100 μL BSS formulation showed 11 and 13% of the injected particles in inferior suprachoroidal space, respectively. One-way ANOVA analysis (equal volume) of BSS and polymeric formulation showed p-values of 0.05, 0.13, 0.0082, 0.020, and 0.023, respectively. Statistically significant differences were found for 2× DCV (50 μL), and 4× DCV (50 μL). Particle weight percent analysis showed a statistically significant amount in opposite to the injection site and posteriorly compared to BSS formulation. HA-based formulation failed to achieve even distribution of particles radially throughout the whole ocular globe. However, significant amounts of particles were delivered from the injection site to 180 degrees away from the injection site.

The hypothesis that strongly non-Newtonian material resisted spreading of embedded particles away from the injection site was tested. The main parameter measured was the suprachoroidal space coverage area. Viscosity of all the polymers was set at approximately 55 Pa-s at a shear rate of 0.1 s⁻¹. This was the viscosity of 90 kDa carboxymethyl-cellulose (12% in water) at 39° C. This was chosen because the 90 kDa carboxymethyl cellulose had a high enough viscosity to be injected through the microneedles and to provide an accurate volume of injection. All of the initial suprachoroidal space coverage areas were measured using ex vivo eyes (Pel-Freez Biologicals).

Immediately after injection, polymeric formulations (700 kDa CMC, 90 kDa CMC, 90 kDa MC) covered suprachoroidal space surface areas of 7-10%. Likewise, BSS formulations showed a suprachoroidal space coverage area of 42%. Initial suprachoroidal space coverage area of the polymeric formulations, which had a viscosity of 55 Pa-s, were 80% smaller than the BSS formulation.

Fourteen days after injection, suprachoroidal space surface coverage area of 700 kDa CMC and 90 kDa MC did not significantly change, but the 90 kDa CMC formulation did. Between days 0 and 14, suprachoroidal space surface coverage area of polymeric formulations (700 kDa CMC, 90 kDa CMC, 90 kDa MC) increased 0.17-4.17 fold. One-way ANOVA showed significant difference in suprachoroidal space coverage area for 90 kDa CMC (p=0.0007), but no statistical difference was found for 700 kDa CMC and 90 kDa MC (p=0.16 and 0.33, respectively). This was expected because 90 kDa CMC showed a lower viscosity increase compared to 700 kDa CMC and 90 kDa MC formulations.

Forty days after injection, suprachoroidal space surface coverage area of 700 kDa and 90 kDa CMC was 12 and 36%. Suprachoroidal space surface coverage area of 90 kDa CMC between 14 and 40 days did not show significant difference (p=0.08 and 0.9, respectively). Sixty days after injection, suprachoroidal space surface coverage area of 700 kDa increased up to 0.2 fold. 700 kDa CMC, between 0 and 60 days, showed a progressive increase up to 2 fold with a statistically significant difference (p=0.001).

Overall, these data show that strongly non-Newtonian fluids at lower shear rate were capable of slowing down the spreading of particles inside the suprachoroidal space for up to 2 months. Higher concentrations of 700 kDa CMC would be expected to be capable of slowing down the spreading of particles for longer periods of time due to higher viscosity at lower shear rate. The strongly non-Newtonian property of 700 kDa CMC allowed reliable injection through microneedles. This is because fluid flowing through the microneedle will experience very high shear stress, which will lower the viscosity of material flowing through the needle. Up to 3 wt % 700 kDa CMC solution was tested and was able to be reliably injected through the microneedles (Data not shown). However, difficulty was experienced injecting reliable volumes using concentrations higher than 12% for 90 kDa CMC.

Suprachoroidal space injection provides access to many unique locations within the ocular globe such as ciliary body and choroid. The micron-sized tip of the microneedles simplifies the delivery into the suprachoroidal space by allowing the tip to just penetrate into, but not across, the suprachoroidal space. Previous research in this area showed microneedles could be used to inject particles as large as 10 μm into the suprachoroidal space. This study built on the previous success of using microneedles to deliver materials into the suprachoroidal space to enhance targeting ability within the suprachoroidal space by controlling the movement of the particles.

Suprachoroidal delivery is a very attractive method to deliver drugs because it allows placement of therapeutics exactly adjacent to the targeted tissues like ciliary body and choroid, which are the sites of action for serious vision-threatening diseases such as glaucoma, wet age-related macular degeneration, diabetic retinopathy, and uveitis. Currently, sustained-release formulations are delivered as an implant that are placed in the vitreous, a chamber at the center of the eye, which often requires surgical procedures to insert the implants.

Microneedles provide a simple and reliable way to deliver polymeric controlled-release formulations in a minimally invasive way. Currently, retinal specialists give millions of intravitreal injections per year at the same site of injection located 2-5 mm from the limbus. This similarity makes the injection procedure straightforward for an ophthalmologist. Suprachoroidal space injection using microneedles also carries fewer safety concerns because the needle only penetrates partially into the eye. On the other hand, intravitreal injection requires the needle to penetrate across the entire outer layer of the eye.

This study demonstrated for the first time that polymeric excipient formulations could be used to target specific regions within the suprachoroidal space using polymeric formulations to control the mobility of polymeric particles. This highly targeted delivery reduces the amount of drug administered. This opens up an opportunity for delivery of longer sustained release formulations, due to reduction in required dosage. This can save money, due to lower drug costs. This can also improve safety and patient acceptance, due to reduced side effects. For example, intravitreal administration of steroids causes unwanted contact with lens and promotes the formation of cataract in 6.6% of the patients. By targeting drug delivery to the targeting site, side effects caused at off-target sites of action can be reduced. Suprachoroidal space delivery could deliver high particle concentrations that could potentially deliver many months of sustained release formulation. In related work accessing the suprachoroidal space, microneedles have been used for hundreds of suprachoroidal injections in rabbits and to a lesser extent in pigs, and were recently reported for use in human subjects. It is believed that the ability to target different regions in the uvea could provide more effective therapies for many vision-threatening diseases.

Many in situ gelling polymers such as solvent removal, temperature, pH, or light mediated introduces potentially toxic materials (organic solvents), and complexities to the procedure. By simply utilizing non-Newtonian fluids to modulate fluid's viscosity at high shear (when flowing through the needle) and low shear rate (when inside the tissue), much simpler pharmaceutical formulations are provided for clinicians use. Polysaccharides provide excellent biocompatibility and are already used in many pharmaceutical formulations. But most importantly, targeting within the suprachoroidal space can be easily achieved by utilizing simple materials that are already approved by FDA for uses in the eye.

While the invention has been described in detail with respect to specific embodiments thereof, it will be appreciated that those skilled in the art, upon attaining an understanding of the foregoing, may readily conceive of alterations to, variations of, and equivalents to these embodiments. Accordingly, the scope of the present invention should be assessed as that of the appended claims and any equivalents thereof.

We claim:

1. A fluid formulation for administration to a suprachoroidal space of an eye of a patient comprising:
particles which comprise a therapeutic agent; and
a non-Newtonian fluid in which the particles are dispersed,
wherein the formulation has a low shear rate viscosity from about 50 to about 275,000 cP and is effective to permit migration of the particles from an insertion site in the suprachoroidal space to a treatment site, which is distal to the insertion site, in the suprachoroidal space and to facilitate localization of the microparticles at the treatment site in the suprachoroidal space.

2. The fluid formulation of claim 1, wherein the non-Newtonian fluid comprises a carboxymethyl cellulose having a molecular weight from about 90 kDa to about 700 kDa.

3. The fluid formulation of claim 1, wherein the non-Newtonian fluid comprises a methylcellulose having a molecular weight from about 50 kDa to about 100 kDa.

4. The fluid formulation of claim 1, wherein the non-Newtonian fluid comprises a hyaluronic acid having a molecular weight from about 100 kDa to about 1000 kDa.

5. The fluid formulation of claim 1, wherein the formulation has a low shear rate viscosity from about 5,000 cP to about 100,000 cP.

6. The fluid formulation of claim 1, wherein the formulation is a thixotropic fluid having a ratio of a low shear rate viscosity to a high shear rate viscosity of at least about 5.

7. The fluid formulation of claim 1, wherein the formulation is a thixotropic fluid having a ratio of a low shear rate viscosity to a high shear rate viscosity of at least about 1000.

8. The fluid formulation of claim 1, wherein the formulation has a viscosity effective to substantially distribute the particles throughout a majority of the suprachoroidal space.

9. The fluid formulation of claim 1, wherein the formulation has a viscosity effective to localize a majority of the particles at the treatment site.

10. The fluid formulation of claim 1, wherein the particles comprise microparticles having an average diameter from about 1 μm to about 50 μm.

11. The fluid formulation of claim 1, wherein the particles comprise nanoparticles having an average diameter from about 1 nm to 999 nm.

12. The fluid formulation of claim 1, wherein the formulation is effective to immobilize a majority of the particles at the treatment site for greater than 2 months.

13. The fluid formulation of claim 1, wherein the formulation is effective to immobilize a majority of the particles at the treatment site for greater than 6 months.

14. A fluid formulation for administration to a suprachoroidal space of an eye of a patient comprising a dispersion of microparticles in a liquid phase, the microparticles comprising a therapeutic agent and a high-density material having a specific gravity of greater than about 1.0.

15. The fluid formulation of claim 14, wherein the microparticles comprises particle-stabilized emulsion droplets.

16. The fluid formulation of claim 15, wherein the particle-stabilized emulsion droplets comprise a liquid core substantially surrounded by a plurality of nanoparticles.

17. The fluid formulation of claim 16, wherein the liquid core comprises fluorocarbon.

18. The fluid formulation of claim 17, wherein the fluorocarbon comprises perflurodecalin.

19. The fluid formulation of claim 16, wherein the plurality of nanoparticles have an average diameter from about 10 nm to about 200 nm.

20. The fluid formulation of claim 14, wherein the high-density material comprises an aggregate of materials which together have a specific gravity of greater than about 1.0.

21. The fluid formulation of any one of claims 14 to 20, wherein the microparticles comprise a biodegradable polymer.

22. A fluid formulation for administration to a suprachoroidal space of an eye of a patient comprising a dispersion of microparticles in a liquid phase, the microparticles comprising a a therapeutic agent and a low-density material having a specific gravity of less than about 1.0.

23. The fluid formulation of claim 22, wherein the microparticles comprises particle-stabilized emulsion droplets.

24. The fluid formulation of claim 23, wherein the particle-stabilized emulsion droplets comprise a liquid or gas core substantially surrounded by a plurality of nanoparticles.

25. The fluid formulation of claim 24, wherein the core of the particle-stabilized emulsion droplets comprises a liquid that is converted into a gas after injection into the eye.

26. The fluid formulation of claim 24, wherein the plurality of nanoparticles have an average diameter from about 10 nm to about 200 nm.

27. The fluid formulation of claim 22, wherein the high-density material comprises an aggregate of materials which together have a specific gravity of less than about 1.0.

28. The fluid formulation of any one of claims 22 to 27, wherein the microparticles comprise a biodegradable polymer.

29. A system comprising the fluid formulation of any one of claims 1 to 28 and one or more microneedles configured to deliver the fluid formulation to the suprachoroidal space of a patient in need of treatment.

30. A method for administering a drug to an eye of a patient comprising:
inserting a microneedle into the eye at an insertion site;
infusing a volume (V) of a drug formulation through the microneedle into the suprachoroidal space of the eye at the insertion site over a first period, wherein the drug formulation comprises particles, a polymeric continuous phase in which the particles are dispersed, and a therapeutic agent which is in the particles and/or in the continuous phase, and wherein the drug formulation has a low shear rate viscosity of from about 50 cP to about 275,000 cP,
wherein during the first period the drug formulation is distributed over a first region which is less than about 10% of the suprachoroidal space, and
wherein during a second period subsequent to the first period the drug formulation is distributed over a second region which is greater than about 20% of the suprachoroidal space.

31. The method of claim 30, wherein the second region is greater than about 50% of the suprachoroidal space.

32. The method of claim 30, wherein the second region is greater than about 75% of the suprachoroidal space.

33. The method of claim 30, wherein the first period is from about 5 seconds to about 10 minutes and the second period is from about 1 day to about 30 days.

34. The method of claim 30, wherein the volume infused is from about 10 to about 500 μL.

35. The method of claim 30, wherein the drug formulation has a low shear rate viscosity of from about 5,000 cP to about 250,000 cP.

36. The method of claim 30, wherein the drug formulation comprises a thixotropic fluid having a ratio of a low shear rate viscosity to a high shear rate viscosity of at least about 5.

37. The method of claim 30, wherein the drug formulation comprises a thixotropic fluid having a ratio of a low shear rate viscosity to a high shear rate viscosity of at least about 1000.

38. The method of claim 30, wherein the drug formulation is characterized by a slope greater than about −10,000 cP/s⁻¹ on a plot of viscosity and shear rate.

39. The method of claim 30, wherein the particles comprise microparticles having an average diameter from about 1 μm to about 50 μm.

40. The method of claim 30, wherein the particles comprise nanoparticles having an average diameter from about 10 nm to about 999 nm.

41. The method of claim 30, wherein the insertion site is at the pars plana region of the eye.

42. The method of claim 30, wherein the therapeutic agent is disposed in the particles.

43. The method of claim 42, wherein greater than about 50% of the particles are delivered to a treatment site within the second region of the suprachoroidal space.

44. The method of claim 42, wherein greater than about 75% of the particles are delivered to the treatment site within the second region of the suprachoroidal space.

45. The method of claim 42, wherein greater than 90% of the particles are delivered to the treatment site within the second region of the suprachoroidal space.

46. The method of claim 30, wherein an effective amount of the therapeutic agent administered by the method is more than about 10 times lower than a comparative effective amount of the therapeutic agent administered topically.

47. The method of claim 30, wherein an effective amount of the therapeutic agent administered by the method is more than about 50 times lower than a comparative effective amount of the therapeutic agent administered topically.

48. The method of claim 30, wherein an effective amount of the therapeutic agent administered by the method is more than about 100 times lower than a comparative effective amount of the therapeutic agent administered topically.

49. A method for administering a drug to an eye of a patient comprising:
inserting a microneedle into the eye at an insertion site;
infusing a drug formulation through the microneedle into the suprachoroidal space of the eye at the insertion site, wherein the drug formulation comprises microparticles dispersed in a liquid phase, the microparticles comprising a high-density material having a specific gravity of greater than or a low-density material having a specific gravity of less than about 1.0; and
directing movement of a majority of the microparticles in the suprachoroidal space to a treatment site by positioning the patient in the gravitational field to direct movement of a majority of the microparticles either upward or downward in the gravitational field, depending on the specific gravity of the microparticles.

50. The method of claim 49, wherein the microparticles comprise a high-density material having a specific gravity of greater than 1.0.

51. The method of claim 50, wherein the fluid formulation is injected into a first region of the eye, and the gravitational field directs movement of the microparticles downward to a second region of the eye posterior to the first region of the eye.

52. The method of claim 50, wherein the fluid formulation is injected into a first region of the eye, and the gravitational field directs movement of the microparticles downward to a second region of the eye anterior to the first region of the eye.

53. The method of claim 49, wherein the microparticles comprise a low-density material having a specific gravity of less than 1.0.

54. The method of claim 53, wherein the fluid formulation is injected into a first region of the eye, and the gravitational field directs movement of the microparticles upward to a second region of the eye posterior to the first region of the eye.

55. The method of claim 53, wherein the fluid formulation is injected into a first region of the eye, and the gravitational field directs movement of the microparticles upward to a second region of the eye anterior to the first region of the eye.

56. The method of any one of claims 49 to 55, wherein the the patient remains positioned in the gravitational field for a time sufficient for the suprachoroidal space to substantially collapse back together again.

57. The method of claim 56, wherein the time sufficient is from about 30 seconds to about one hour.

58. A method for treating uveitis by administering the drug formulation to an eye of a patient using the method of any one of claims 30 to 57.

59. The method of claim 58, wherein the uveitis is chronic.

60. The method of claim 58, wherein the uveitis is acute.

61. A method for treating retinal vein occlusion by administering the drug formulation to an eye of a patient using the method of any one of claims 30 to 57.

62. A method for treating macular edema by administering the drug formulation to an eye of a patient using the method of any one of claims 30 to 57.

63. The method of claim 62, wherein the macular edema is associated with uveitis.

64. The method of claim 63, wherein the uveitis is chronic.

65. The method of claim 63, wherein the uveitis is acute.

66. The method of claim 60, wherein the macular adema is associated with retinal vein occlusion.

67. The method of claim 60, wherein the drug formulation comprises an anti-inflammatory agent.

68. The method of claim 66, wherein the method further comprises injecting a VEGF modulator intravitreally.

69. A method for treating wet AMD by administering the drug formulation to an eye of a patient using the method of any one of claims 30 to 57.

70. A method for treating dry AMD by administering the drug formulation to an eye of a patient using the method of any one of claims 30 to 57.

71. A method for treating glaucoma by administering a drug formulation to an eye of a patient comprising:
inserting a microneedle into the eye at an insertion site in an anterior portion of the eye;
infusing a volume (V) of a drug formulation through the microneedle into the suprachoroidal space of the eye at the insertion site, wherein the drug formulation comprises particles, a polymeric continuous phase in which the particles are dispersed, and a therapeutic agent which is in the particles and/or in the continuous phase, and wherein the drug formulation has a low shear rate viscosity of greater than about 10,000 cP,
wherein the drug formulation is substantially localized at the insertion site after being infused into the suprachoroidal space.

72. The method of claim 71, wherein the therapeutic agent is an anti-glaucoma agent selected from the group consisting of prostaglandins, beta-blockers, alpha-adrenergic agonists, carbonic anhydrase inhibitors, parasympathomimetics, epinephrine, and combinations thereof.

73. The method of claim 71, wherein an effective amount of the therapeutic agent administered by the method is more than about 10 times lower than a comparative effective amount of the therapeutic agent administered topically.

74. The method of claim 71, wherein an effective amount of the therapeutic agent administered by the method is more than about 50 times lower than a comparative effective amount of the therapeutic agent administered topically.

75. The method of claim 71, wherein an effective amount of the therapeutic agent administered by the method is more than about 100 times lower than a comparative effective amount of the therapeutic agent administered topically.

76. The method of claim 71, wherein the administration of the drug formulation is non-surgical.

77. The method of claim 71, wherein the particles comprise microparticles, nanoparticles, or a combination thereof.
